SEQUENCE LISTING (3 sequences)

SEQ ID NO: 1 - protein, 175 aa, Artificial Sequence
  Description of Artificial Sequence Synthetic Polypeptide
  VARIANT (1): N-term Met may or may not be present
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 175 aa, Artificial Sequence
  Description of Artificial Sequence Synthetic Polypeptide
  VARIANT (1): N-term Met may or may not be present
MAPTYRASSLPQSFLLKSLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 204 aa, Artificial Sequence
  Description of Artificial Sequence Synthetic Polypeptide
  VARIANT (1): N-term Met may or may not be present
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2007/0092482 A1
(43) Pub. Date: Apr. 26, 2007

(54) Conjugates of a G-CSF moiety and a polymer

(76) Inventors: Mary J. Bossard, Madison, AL (US); Ping Zhang, Madison, AL (US)

Correspondence Address: NEKTAR THERAPEUTICS, 150 INDUSTRIAL ROAD, SAN CARLOS, CA, 94070, US

(21) Appl. No.: 11/499,115

(22) Filed: Aug. 4, 2006

Related U.S. Application Data
(60) Provisional application No. 60/705,968, filed on Aug. 4, 2005.
(60) Provisional application No. 60/752,825, filed on Dec. 21, 2005.

(51) Int. Cl. A61K 38/19; C07K 14/53

(52) U.S. Cl. 424/78.27; 424/85.1; 525/54.1; 530/351

(57) ABSTRACT

Conjugates of a G-CSF moiety and one or more nonpeptidic water-soluble polymers are provided. Typically, the nonpeptidic water-soluble polymer is poly(ethylene glycol) or a derivative thereof. Also provided, among other things, are compositions comprising conjugates, methods of making conjugates, and methods of administering compositions comprising conjugates to a patient.

### CROSS-REFERENCE TO RELATED APPLICATIONS

This application claims the benefit of priority to the following U.S. provisional patent applications: U.S. Provisional Patent Application Ser. No. 60/752,825, filed on Dec. 21, 2005; and U.S. Provisional Patent Application Ser. No. 60/705,968, filed on Aug. 4, 2005, the contents of which are incorporated herein by reference in their entireties.

### FIELD OF THE INVENTION

Among other things, one or more embodiments of the present invention relate generally to conjugates comprising a G-CSF moiety (i.e., a moiety having at least some granulocyte-colony stimulating factor activity) and a polymer. In addition, the invention relates to (among other things) compositions comprising conjugates, methods for synthesizing conjugates, and methods of administering a composition.

### BACKGROUND OF THE INVENTION

One important function of the human hematopoeitic system is the replacement of a variety of white blood cells (including macrophages, neutrophils, and basophils/mast cells), red blood cells (erythrocytes) and clot-forming cells (megakaryocytes/platelets). Each of these specialized cells is formed from hematopoeitic precursor cells located in the bone marrow. Specific hormone-like glycoproteins called “colony stimulating factors” control the differentiation and maturation of the hematopoeitic precursor cells into any one of the specialized blood cells.

One such colony stimulating factor is granulocyte-colony stimulating factor or “G-CSF.” As its name implies, this colony stimulating factor promotes the proliferation and differentiation of granulocytes, although G-CSF can promote the formation of other cell types as well. G-CSF is produced by a number of different cell types (including activated T cells, B cells, macrophages, mast cells, endothelial cells and fibroblasts) in response to cytokine, immune and/or inflammatory stimuli. Native human G-CSF is a glycoprotein of 174 amino acids and can have a variety of molecular weights depending on the extent of glycosylation. The molecular weight of human G-CSF is approximately 19,000.

Pharmacologically, G-CSF has been administered to cancer patients receiving chemotherapy treatments so that white blood cells killed during these treatments are more quickly replaced. With a similar aim of accelerating white blood cell replacement, administration of G-CSF is used in the treatment of leukemia patients undergoing bone marrow replacement therapy. Additional uses of G-CSF, such as accelerated wound healing, have been proposed. See, for example, U.S. Pat. No. 6,689,351.

One drawback associated with G-CSF therapy is frequency of dosing. Because G-CSF therapy typically requires daily injections, patients dislike the inconvenience and discomfort associated with this regimen. Coupled with the fact that patients require frequent blood testing to determine white blood cells counts (which require trips to a health care practitioner), many patients would prefer an alternative that is less cumbersome and/or involves a reduction in the number of injections.

One proposed solution to these problems has been to provide a prolonged release form of G-CSF. For example, U.S. Pat. No. 5,942,253 describes microspheres of poly(lactic acid-co-glycolic acid) or other biodegradable polymers of G-CSF. The formation of microspheres, however, can be a complex process, requiring several synthetic steps. Thus, this prolonged release approach suffers from synthetic complexities that are ideally avoided.

PEGylation, or the attachment of a poly(ethylene glycol) derivative to a protein, has been described as a means to prolong a protein's in vivo half-life, thereby resulting in prolonged pharmacologic activity. For example, U.S. Pat. No. 5,880,255 describes a conjugate of G-CSF and poly(ethylene glycol) formed from a reaction with 2,2,2-trifluoroethanesulfonate derivatized linear monomethoxy poly(ethylene glycol) having a molecular weight of 5,000 Daltons.

U.S. Pat. No. 6,646,110 describes certain conjugates wherein the G-CSF protein is altered by 1 to 15 amino acid residues comprising an attachment group for a non-polypeptide moiety, and having at least one non-polypeptide moiety attached to an attachment group of the protein.

U.S. Pat. No. 6,166,183 describes conjugates formed from the reaction of G-CSF and certain polymeric reagents (e.g., mPEG-succinimidyl propionate and certain mPEG triazine derivatives). U.S. Pat. No. 6,027,720 also describes conjugates formed from the reaction of G-CSF and certain MPEG triazine derivatives.

Two publications discuss the attachment of certain polymeric reagents to an internal cysteine residue of G-CSF. Although the conjugation methods described in these methods are different, each method suffers from at least one significant drawback. U.S. Patent Application Publication No. 2005/0143563 requires relatively harsh conditions that can cause precipitation of aggregates. International Patent Publication No. 05/099769 describes a process requiring the induction of reversible denaturation of G-CSF.

A commercial product of a PEGylated G-CSF is available from Amgen Inc. (Thousand Oaks CA) under the name NEULASTA® and is a covalent conjugate of recombinant methionyl human G-CSF (filgrastim) and monomethoxypolyethylene glycol.

Notwithstanding these conjugates, however, there remains a need for other conjugates of G-CSF that have different structures. Among other things, one or more embodiments of the present invention is therefore directed to such conjugates as well as compositions comprising the conjugates and related methods as described herein, which are believed to be new and completely unsuggested by the art.

### SUMMARY OF THE INVENTION

Accordingly, a conjugate is provided, the conjugate comprising a G-CSF moiety covalently attached, either directly or through a spacer moiety, to a nonpeptidic water-soluble polymer. The conjugate is typically provided as part of a composition.

In one or more embodiments of the invention, a conjugate is provided, the conjugate comprising a residue of a G-CSF precursor moiety covalently attached, either directly or through a spacer moiety comprised of one or more atoms, to a water-soluble polymer. The attachment site of the polymer can be located at any point on the G-CSF precursor moiety and can be on the portion that is required for activity following in vivo cleavage of the precursor form. In addition, the attachment site of the polymer can be located on the portion that has no G-CSF activity following cleavage of the precursor form.

In one or more embodiments of the invention, a conjugate is provided, the conjugate comprising a water soluble polymer covalently attached to a G-CSF moiety via a cysteine residue of the G-CSF moiety.

In one or more embodiments of the invention, a conjugate is provided, the conjugate comprising a residue of a G-CSF moiety having a cysteine residue side chain, wherein the cysteine residue side chain is attached, either directly or through a spacer moiety comprised of one or more atoms, to a water-soluble polymer

In one or more embodiments of the invention, a conjugate is provided, the conjugate comprising a residue of a G-CSF moiety having a cysteine residue side chain that is not involved in a disulfide bond in unconjugated form, wherein the cysteine residue side chain is attached, either directly or through a spacer moiety comprised of one or more atoms, to a water-soluble polymer.

In one or more embodiments of the invention, a conjugate is provided, the conjugate comprising a residue of a G-CSF moiety having a cysteine residue side chain corresponding to amino acid position 17 of hG-CSF, wherein the cysteine residue side chain is attached, either directly or through a spacer moiety comprised of one or more atoms, to a water-soluble polymer.

In one or more embodiments of the invention, a conjugate is provided, the conjugate comprising a residue of a G-CSF moiety attached through an amide or a secondary amine linkage to a branched water-soluble polymer, wherein (i) an optional spacer moiety comprised of one or more atoms is located between the amide or secondary amine linkage and the branched water-soluble polymer, and (ii) the branched water-soluble polymer does not contain a lysine residue.

In one or more embodiments of the invention, a conjugate is provided, the conjugate comprising a residue of a G-CSF moiety covalently attached via a degradable linkage, either directly or through a spacer moiety comprised of one or more atoms, to a water-soluble polymer. Preferably, the degradable linkage is a cleavable degradable linkage and is “tagless,” meaning that upon degradation and cleavage of the polymer from the G-CSF moiety, the original or native G-CSF moiety is generated, without any additional atoms or residue (i.e., a “tag”) of the polymeric reagent attached to the G-CSF moiety.

In one or more embodiments of the invention, a composition is provided, the composition comprising a plurality of conjugates, each conjugate comprising a residue of a G-CSF moiety attached, either directly or through a spacer moiety comprised of one or more atoms, to a water-soluble polymer, wherein less than 50% of all conjugates in the composition are N-terminally monoPEGylated.

In one or more embodiments of the invention, a conjugate is provided, the conjugate comprising the following structure:
POLY″−(X²)_{b}−POLY′−(X¹)ₐ−(G-CSF)
wherein:
POLY″ is a second water-soluble polymer (preferably branched or linear);
POLY′ is a first water-soluble polymer (preferably linear);
X¹, when present, is first spacer moiety comprised of one or more atoms;
X², when present, is a second spacer moiety comprised of one or more atoms;
(b) is either zero or one;
(a) is either zero or one; and
G-CSF is a residue of a G-CSF moiety.

In one or more embodiments of the invention, a method for preparing a conjugate is provided, the method comprising adding a polymeric reagent composition to a G-CSF moiety composition under conditions sufficient to result in a conjugate composition comprising a residue of a G-CSF moiety covalently attached, either directly or through a spacer moiety comprised of one or more atoms, to a water-soluble polymer.

In one or more embodiments of the invention, a method for preparing a conjugate is provided, the method comprising adding a first polymeric reagent composition to a G-CSF moiety composition under conditions sufficient to result in a first conjugate composition comprising a first conjugate comprised of a residue of a G-CSF moiety covalently attached, either directly or through a first spacer moiety comprised of one or more atoms, to a first water-soluble polymer, and adding a second polymeric reagent composition to the first conjugate composition to result in a second conjugate composition comprising a second water-soluble polymer attached, either directly or through a second spacer moiety comprised of one or more atoms, to the first water-soluble polymer of the conjugate.

In one or more embodiments of the invention, a method for preparing a conjugate is provided, the method comprising combining a polymeric reagent and a G-CSF moiety under conditions sufficient to result in the formation of a conjugate comprising a residue of a G-CSF moiety covalently attached, either directly or through a spacer moiety comprised of one or more atoms, to a water-soluble polymer, wherein the G-CSF moiety is covalently attached at a side chain of a cysteine residue, and further wherein the method (a) lacks a step introducing denaturing conditions, and (b) is carried out at a pH of less than 8.5 or lacks a step of adding a detergent.

In one or more embodiments of the invention, a method for delivering a conjugate to a patient is provided, the method comprising the step of administering to the patient a composition comprising a conjugate as described herein, wherein the composition contains a therapeutically effective amount of one or more of the conjugates. The step of administering the conjugate can be effected by injection (e.g., intramuscular injection, intravenous injection, subcutaneous injection, and so forth).

### BRIEF DESCRIPTION OF THE DRAWINGS

FIG. 1 is a plot corresponding to a composition prepared in Example 1A.

FIG. 2 is a copy of a gel resulting from SDS-PAGE analysis of a composition prepared in Example 1A.

FIG. 3 is a plot corresponding to a composition prepared in Example 1A.

FIG. 4 is a plot corresponding to a composition prepared in Example 1B.

FIG. 5 is a copy of a gel resulting from SDS-PAGE analysis of a composition prepared in Example 1B.

FIG. 6 is a plot corresponding to a composition prepared in Example 1C.

FIG. 7 is a plot corresponding to a composition prepared in Example 1D.

FIG. 8 is a copy of a gel resulting from SDS-PAGE analysis of compositions prepared in Example 2A.

FIG. 9 is a plot corresponding to a composition prepared in Example 2A.

FIG. 10 is a plot corresponding to a composition prepared in Example 2B.

FIG. 11 and FIG. 12 are a plots corresponding to samples prepared in Example 3A.

FIG. 13 is a plot corresponding to a sample prepared in Example 3B.

FIG. 14 is a copy of a gel resulting from SDS-PAGE analysis of compositions prepared in Examples 4 and 5.

FIG. 15 is a plot showing the release profile of a conjugate as described in Example 4.

FIG. 16 is a plot showing the hydrolysis rate of a conjugate as described in Example 4.

FIG. 17 is a plot showing the release of a conjugate as described in Example 5.

FIG. 18 is a plot showing the hydrolysis rate of a conjugate as described in Example 5.

FIG. 19 is a plot corresponding to a composition prepared in Example 6.

FIG. 20 and FIG. 21 are plots showing the activity of various PEG-G-CSF conjugates at 48 hours and 72 hours, respectively, as described in Example 9.

FIG. 22, FIG. 23, FIG. 24, FIG. 25, FIG. 26, FIG. 27, FIG. 28, and FIG. 29 are each plots showing either neutrophil response or white blood cell count of various PEG-G-CSF conjugates, as described in Example 9.

### DETAILED DESCRIPTION OF THE INVENTION

Before describing one or more embodiments of the present invention in detail, it is to be understood that this invention is not limited to the particular polymers, synthetic techniques, G-CSF moieties, and the like, as such may vary.

It must be noted that, as used in this specification and the intended claims, the singular forms “a,” “an,” and “the” include plural referents unless the context clearly dictates otherwise. Thus, for example, reference to “a polymer” includes a single polymer as well as two or more of the same or different polymers, reference to “an optional excipient” refers to a single optional excipient as well as two or more of the same or different optional excipients, and the like.

In describing and claiming one or more embodiments of the present invention, the following terminology will be used in accordance with the definitions described below.

“PEG,” “polyethylene glycol” and “poly(ethylene glycol)” as used herein, are interchangeable and meant to encompass any nonpeptidic, water-soluble poly(ethylene oxide). Typically, PEGs for use in accordance with the invention comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃O(CH₂CH₂O)ₙ— and CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled can be determined by using a suitable detector. Such labels include, without limitation, fluorescers, chemiluminescers, moieties used in enzyme labeling, calorimetric moieties (e.g., dyes), metal ions, radioactive moieties, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

“Non-naturally occurring” with respect to a polymer as described herein, means a polymer that in its entirety is not found in nature. A non-naturally occurring polymer of the invention may, however, contain one or more monomers or segments of monomers that are naturally occurring, so long as the overall polymer structure is not found in nature.

The term “water soluble” as in a “water-soluble polymer” is any polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

Molecular weight in the context of a water-soluble polymer of the invention, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

The terms “active” or “activated” when used in conjunction with a particular functional group, refer to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

The terms “spacer moiety,” “linkage” and “linker” are used herein to refer to an atom or a collection of atoms optionally used to link interconnecting moieties such as a terminus of a polymer segment and a G-CSF moiety or an electrophile or nucleophile of a G-CSF moiety. The spacer moiety may be hydrolytically stable or may include a physiologically hydrolyzable or enzymatically degradable linkage.

“Alkyl” refers to a hydrocarbon chain, typically ranging from about 1 to 15 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl as well as cycloalkylene-containing alkyl.

“Lower alkyl” refers to an alkyl group containing from 1 to 6 carbon atoms, and may be straight chain or branched, as exemplified by methyl, ethyl, n-butyl, i-butyl, and t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8 carbon atoms. “Cycloalkylene” refers to a cycloalkyl group that is inserted into an alkyl chain by bonding of the chain at any two carbons in the cyclic ring system.

“Alkoxy” refers to an —O—R group, wherein R is alkyl or substituted alkyl, preferably C₁₋₆ alkyl (e.g., methoxy, ethoxy, propyloxy, and so forth).

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; alkoxy, lower phenyl; substituted phenyl; and the like. “Substituted aryl” is aryl having one or more noninterfering groups as a substituent. For substitutions on a phenyl ring, the substituents may be in any orientation (i.e., ortho, meta, or para).

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen.

“Substituted heteroaryl” is heteroaryl having one or more noninterfering groups as substituents.

“Substituted heterocycle” is a heterocycle having one or more side chains formed from noninterfering substituents.

“Electrophile” and “electrophilic group” refer to an ion or atom or collection of atoms, that may be ionic, having an electrophilic center, i.e., a center that is electron seeking, capable of reacting with a nucleophile.

“Nucleophile” and “nucleophilic group” refers to an ion or atom or collection of atoms that may be ionic having a nucleophilic center, i.e., a center that is seeking an electrophilic center or with an electrophile.

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Pharmaceutically acceptable excipient or carrier” refers to an excipient that may optionally be included in the compositions of the invention and that causes no significant adverse toxicological effects to the patient. “Pharmacologically effective amount,” “physiologically effective amount,” and “therapeutically effective amount” are used interchangeably herein to mean the amount of a polymer-(G-CSF) moiety conjugate that is needed to provide a desired level of the conjugate (or corresponding unconjugated G-CSF moiety) in the bloodstream or in the target tissue. The precise amount will depend upon numerous factors, e.g., the particular G-CSF moiety, the components and physical characteristics of the therapeutic composition, intended patient population, individual patient considerations, and the like, and can readily be determined by one skilled in the art, based upon the information provided herein.

“Multi-functional” means a polymer having three or more functional groups contained therein, where the functional groups may be the same or different. Multi-functional polymeric reagents of the invention will typically contain a number of functional groups satisfying one or more of the following ranges: from about 3-100 functional groups; from 3 to 50 functional groups; from 3 to 25 functional groups; from 3 to 15 functional groups; and from 3 to 10 functional groups; exemplary numbers of functional groups include 3, 4, 5, 6, 7, 8, 9 and 10 functional groups within the polymeric reagent.

The term “G-CSF moiety,” as used herein, refers to a moiety having G-CSF activity, and, unless the context clearly dictates otherwise, also refers to a G-CSF precursor moiety (an exemplary sequence of which is provided in SEQ ID NO: 3). The G-CSF moiety will also have at least one electrophilic group or nucleophilic group suitable for reaction with a polymeric reagent. In addition, the term “G-CSF moiety” encompasses both the G-CSF moiety prior to conjugation as well as the G-CSF moiety residue following conjugation. As will be explained in further detail below, one of ordinary skill in the art can determine whether any given moiety has G-CSF activity. Proteins comprising an amino acid sequence corresponding to any one of SEQ ID NOS: 1 through 2 is a G-CSF moiety, as well as any protein or polypeptide substantially homologous thereto, whose biological properties result in the stimulation of growth and/or number of neutrophils and/or activity similar to G-CSF. As used herein, the term “G-CSF moiety” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. These terms include both natural and recombinantly produced G-CSF.

The term “substantially homologous” means that a particular subject sequence, for example, a mutant sequence, varies from a reference sequence by one or more substitutions, deletions, or additions, the net effect of which does not result in an adverse functional dissimilarity between the reference and subject sequences. For purposes of the present invention, sequences having greater than 95 percent homology, equivalent biological properties, and equivalent expression characteristics are considered substantially homologous. For purposes of determining homology, truncation of the mature sequence should be disregarded. Sequences having lesser degrees of homology, comparable bioactivity, and equivalent expression characteristics are considered substantial equivalents. Exemplary G-CSF moieties for use herein include those sequences that are substantially homologous SEQ ID NO: 1.

The term “fragment” of the G-CSF protein means any protein or polypeptide having the amino acid sequence of a portion or fragment of a G-CSF protein, and which has the biological activity of the G-CSF. Fragments include proteins or polypeptides produced by proteolytic degradation of the G-CSF protein or produced by chemical synthesis by methods routine in the art. A G-CSF protein or fragment thereof is biologically active when administration of the protein or fragment to a human results in some degree of G-CSF activity. Determining such biological activity of the G-CSF protein can carried out by conventional, well known tests utilized for such purposes on one or more species of mammals. An appropriate test which can be utilized to demonstrate such biological activity is described herein.

The term “patient,” refers to a living organism suffering from or prone to a condition that can be prevented or treated by administration of an active agent (e.g., conjugate), and includes both humans and animals.

“Optional” or “optionally” means that the subsequently described circumstance may or may not occur, so that the description includes instances where the circumstance occurs and instances where it does not.

“Substantially” means nearly totally or completely, for instance, satisfying one or more of the following: greater than 50%, 51% or greater, 75% or greater, 80% or greater, 90% or greater, and 95% or greater of the condition.

Unless the context clearly dictates otherwise, when the term “about” precedes a numerical value, the numerical value is understood to mean ±10% of the stated numerical value.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutarnine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

Turning to one or more embodiments of the invention, a conjugate is provided, the conjugate comprising a G-CSF moiety covalently attached, either directly or through a spacer moiety, to a nonpeptidic water-soluble polymer. The conjugates of the invention will have one or more of the following features.

The G-CSF Moiety

As previously stated, the conjugate generically comprises a G-CSF moiety covalently attached, either directly or through a spacer moiety, to a nonpeptidic water-soluble polymer. As used herein, the term “G-CSF moiety” shall refer to the G-CSF moiety prior to conjugation as well as to the G-CSF moiety following attachment to a nonpeptidic water-soluble polymer. It is understood, however, that when the G-CSF moiety is attached to a nonpeptidic water-soluble polymer, the G-CSF moiety is slightly altered due to the presence of one or more covalent bonds associated with linkage to the polymer. Often, this slightly altered form of the G-CSF moiety attached to another molecule is referred to a “residue” of the G-CSF moiety. The G-CSF moiety in the conjugate can be any moiety that provides a granulocyte-colony stimulating factor effect.

The G-CSF moiety can be derived from either non-recombinant methods or from recombinant methods and the invention is not limited in this regard. In addition, the G-CSF moiety can be derived from human sources or from animal sources.

The G-CSF moiety can be derived non-recombinantly. For example, as described in U.S. Pat. No. 4,810,643, it is possible to collect G-CSF from the culture medium of a human carcinoma cell line denominated 5637 and deposited under restrictive conditions with the American Type Culture Collection, Rockville Md. as A.T.C.C. Deposit No. HTB-9.

The G-CSF moiety can be derived from recombinant methods and can be expressed in bacterial (e.g., E. coli), mammalian (e.g., Chinese hamster ovary cells), and yeast (e.g., Saccharomyces cerevisiae) expression systems. The expression can occur via exogeneous expression or via endogenous expression. For example, Nagata et al. (1986) Nature 319:415 provides the cDNA for human G-CSF (“hG-CSF”) isolated from human squamous cell carcinoma cell line CHU-II and also describes a process for expressing of the protein in COS cells (African Green Monkey cells). Souza et al. describes a process for expressing G-CSF in E. coli cells. U.S. Pat. No. 4,810,643 describes recombinant-based methods for preparing methionyl G-CSF (i.e., G-CSF to which the N-terminus has the amino acid methionine attached). In addition, U.S. Pat. No. 5,633,352 describes recombinant methods for preparing G-CSF.

The amino acid sequence for human G-CSF is provided in SEQ ID NO: 1. As provided therein, a methionine residue-containing form (wherein n′″=1) is also contemplated for this, and all other sequences, described herein. SEQ ID NO 2 corresponds to G-CSF moiety having a different sequence than SEQ ID NO 1.

Although recombinant-based methods for preparing proteins can differ, recombinant methods typically involve constructing the nucleic acid encoding the desired polypeptide or fragment, cloning the nucleic acid into an expression vector, transforming a host cell (e.g., plant, bacteria, yeast, transgenic animal cell, or mammalian cell such as Chinese hamster ovary cell or baby hamster kidney cell), and expressing the nucleic acid to produce the desired polypeptide or fragment. Methods for producing and expressing recombinant polypeptides in vitro and in prokaryotic and eukaryotic host cells are known to those of ordinary skill in the art.

To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding. Fusion proteins can be identified and purified by first running a mixture containing the fusion protein through an affinity column bearing binding moieties (e.g., antibodies) directed against the epitope tag or other binding sequence in the fusion proteins, thereby binding the fusion protein within the column. Thereafter, the fusion protein can be recovered by washing the column with the appropriate solution (e.g., acid) to release the bound fusion protein. The recombinant polypeptide can also be identified and purified by lysing the host cells, separating the polypeptide, e.g., by size exclusion chromatography, and collecting the polypeptide. These and other methods for identifying and purifying recombinant polypeptides are known to those of ordinary skill in the art. In one or more embodiments of the invention, however, it is preferred that the G-CSF moiety is not in the form of a fusion protein.

Depending on the system used to express proteins having G-CSF activity, the G-CSF moiety can be unglycosylated or glycosylated and either may be used. That is, the G-CSF moiety can be unglycosylated or the G-CSF moiety can be glycosylated. In one or more embodiments of the invention, it is preferred that the G-CSF moiety is not glycosylated.

The G-CSF moiety can advantageously be modified to include one or more amino acid residues such as, for example, lysine, cysteine and/or arginine, in order to provide facile attachment of a polymer to an atom within the side chain of the amino acid. In addition, the G-CSF moiety can be modified to include a non-naturally occurring amino acid residue. Techniques for adding amino acid residues and non-naturally occurring amino acid residues are well known to those of ordinary skill in the art. Reference is made to J. March, Advanced Organic Chemistry: Reactions Mechanisms and Structure, 4th Ed. (New York: Wiley-Interscience, 1992). In one or more embodiments of the invention, it is preferred that the G-CSF moiety is not modified to include one or more amino acid residues. Exemplary G-CSF moieties having at least one substitution relative to hG-CSF are provided in U.S. Pat. No. 6,646,110, and are suited for use as a G-CSF moiety herein. Further, exemplary G-CSF moieties having at least one substitution relative to hG-CSF are provided in U.S. Pat. Nos. 6,004,548 and 5,580,755, and are suited for use as a G-CSF moiety herein.

In addition, the G-CSF moiety can advantageously be modified to include attachment of a functional group (other than through addition of a functional group-containing amino acid residue). For example, the G-CSF moiety can be modified to include a thiol group. In addition, the G-CSF moiety can be modified to include an N-terminal alpha carbon. In addition, the G-CSF moiety can be modified to include one or more carbohydrate moieties. In some embodiments of the invention, it is preferred that the G-CSF moiety is not modified to include a thiol group and/or an N-terminal alpha carbon. G-CSF moieties containing an aminoxy, aldehyde or some other functional group can be used.

A preferred G-CSF moiety has an amino acid sequence selected from the group consisting of SEQ ID NO: 1 and SEQ ID NO: 2. Unless specifically noted, all assignments of a numeric location of an amino acid residue as provided herein are based on SEQ ID NO: 1 (ignoring any leading methionyl residue). Sequences that are useful to serve as G-CSF moieties include those sequences of the proteins found in commercially available versions G-CSF-containing formulations such as NEUPOGEN® G-CSF (Amgen, Thousand Oaks, Calif.) and GRASTIM® G-CSF (Dr. Reddy's, Hyderabad, India).

hG-CSF moiety (as provided in SEQ ID NO: 1) can be used as well as truncated versions, hybrid variants, and peptide mimetics of the sequence. Biologically active fragments, deletion variants, substitution variants or addition variants of any of the foregoing that maintain at least some degree of G-CSF activity can also serve as a G-CSF moiety.

For any given peptide or protein moiety, it is possible to determine whether that moiety has G-CSF activity. For example, as described in U.S. Pat. No. 5,580,755, it is possible to administer a G-CSF moiety of interest with buffer into the blood stream of a hamster and count the granulocytes. The G-CSF moiety of interest can serve as an G-CSF moiety in accordance with the present invention if the hamster injected with the proposed G-CSF moiety exhibits a statistically significant increase in granulocytes when compared to a control hamster not injected with the proposed G-CSF moiety (e.g., simply buffer).

The Water-Soluble Polymer (e.g., POLY″, POLY′, POLY¹, POLY², and so forth)

As previously discussed, each conjugate comprises a G-CSF moiety attached to a water-soluble polymer. With respect to the water-soluble polymer, the water-soluble polymer is nonpeptidic, nontoxic, non-naturally occurring and biocompatible. With respect to biocompatibility, a substance is considered biocompatible if the beneficial effects associated with use of the substance alone or with another substance (e.g., an active agent such as a G-CSF moiety) in connection with living tissues (e.g., administration to a patient) outweighs any deleterious effects as evaluated by a clinician, e.g., a physician. With respect to nonimmunogenicity, a substance is considered nonimmunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the nonpeptidic water-soluble polymer is biocompatible and nonimmunogenic.

Further, the polymer is typically characterized as having from 2 to about 300 termini. Examples of such polymers include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and combinations of any of the foregoing.

The polymer is not limited to a particular structure and can be linear (e.g., alkoxy PEG or bifunctional PEG), branched or multi-armed (e.g., forked PEG or PEG attached to a polyol core), and/or dendritic, wherein each of the foregoing can include non-degradable or degradable linkages. Moreover, the internal structure of the polymer can be organized in any number of different patterns and can be selected from the group consisting of homopolymer, alternating copolymer, random copolymer, block copolymer, alternating tripolymer, random tripolymer, and block tripolymer.

Typically, activated PEG and other activated water-soluble polymers (i.e., polymeric reagents) are activated with a suitable activating group appropriate for coupling to a desired site on the G-CSF moiety. Thus, a polymeric reagent will possess a reactive group for reaction with the G-CSF moiety. Representative polymeric reagents and methods for conjugating these polymers to an active moiety are known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182.

Typically, the weight-average molecular weight of the water-soluble polymer in the conjugate is from about 100 Daltons to about 150,000 Daltons. Exemplary ranges, however, include weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. For any given water-soluble polymer, PEGs having a molecular weight in one or more of these ranges are preferred.

Exemplary weight-average molecular weights for the water-soluble polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used. In one or more embodiments, the conjugate will not have any PEG moieties attached, either directly or indirectly, with a PEG having a weight average molecular weight of less than about 6,000 Daltons.

When used as the polymer, PEGs will typically comprise a number of (OCH₂CH₂) monomers [or (CH₂CH₂O) monomers, depending on how the PEG is defined]. As used throughout the description, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

When end-capped polymers are required, a polymer having at least one terminus capped with a relatively inert group, such as a lower C₁₋₆ alkoxy group (although a hydroxyl group) can be used. When the polymer is PEG, for example, it is preferred to use a methoxy-PEG (commonly referred to as MPEG), which is a linear form of PEG wherein one terminus of the polymer has a methoxy (—OCH₃) group, while the other terminus is a hydroxyl or other functional group that can be optionally chemically modified.

In one form useful in one or more embodiments of the present invention, free or unbound PEG is a linear polymer terminated at each end with hydroxyl groups:
HO—CH₂CH₂O—(CH₂CH₂O)ₙ—CH₂CH₂—OH,
wherein (n) typically ranges from zero to about 4,000.

The above polymer, alpha-, omega-dihydroxylpoly(ethylene glycol), can be represented in brief form as HO-PEG-OH where it is understood that the -PEG-symbol can represent the following structural unit:
—CH₂CH₂O—(CH₂CH₂O)ₙ—CH₂CH₂—,
wherein (n) is as defined as above.

Another type of PEG useful in one or more embodiments of the present invention is methoxy-PEG-OH, or MPEG in brief, in which one terminus is the relatively inert methoxy group, while the other terminus is a hydroxyl group. The structure of mPEG is given below.
CH₃O—CH₂CH₂O—(CH₂CH₂O)ₙ—CH₂CH₂—OH
wherein (n) is as described above.

Multi-armed or branched PEG molecules, such as those described in U.S. Pat. No. 5,932,462, can also be used as the PEG polymer. For example, PEG can have the structure: 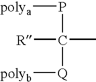
wherein:

polyₐ and poly_{b} are PEG backbones (either the same or different), such as methoxy poly(ethylene glycol);

R″ is a nonreactive moiety, such as H, methyl or a PEG backbone; and

P and Q are nonreactive linkages. In a some circumstances, the branched PEG polymer is methoxy poly(ethylene glycol) disubstituted lysine (e.g., a polymer comprising the following
structure 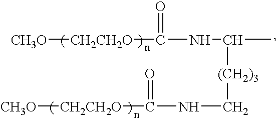
wherein each n is an integer from 3 to 4,000). See, for example, U.S. Pat. No. 5,932,462. Depending on the specific G-CSF moiety used, the reactive ester functional group of the disubstituted lysine may be further modified to form a functional group suitable for reaction with the target group within the G-CSF moiety.

In addition, the PEG can comprise a forked PEG. An example of a forked PEG is represented by the following structure: 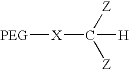
wherein: X is a spacer moiety of one or more atoms and each Z is an activated terminal group linked to CH by a chain of atoms of defined length. U.S. Pat. No. 6,362,254 discloses various forked PEG structures capable of use in one or more embodiments of the present invention. The chain of atoms linking the Z functional groups to the branching carbon atom serve as a tethering group and may comprise, for example, alkyl chains, ether chains, ester chains, amide chains and combinations thereof.

The PEG polymer may comprise a pendant PEG molecule having reactive groups, such as carboxyl, covalently attached along the length of the PEG rather than at the end of the PEG chain. The pendant reactive groups can be attached to the PEG directly or through a spacer moiety, such as an alkylene group.

In addition to the above-described forms of PEG, the polymer can also be prepared with one or more weak or degradable linkages in the polymer, including any of the above-described polymers. For example, PEG can be prepared with ester linkages in the polymer that are subject to hydrolysis. As shown below, this hydrolysis results in cleavage of the polymer into fragments of lower molecular weight:

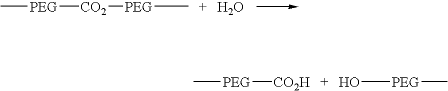

Other hydrolytically degradable linkages, useful as a degradable linkage within a polymer backbone, include: carbonate linkages; imine linkages resulting, for example, from reaction of an amine and an aldehyde (see, e.g., Ouchi et al. (1997) Polymer Preprints 38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

Such optional features of the conjugate, i.e., the introduction of one or more degradable linkages into the polymer chain, may provide for additional control over the final desired pharmacological properties of the conjugate upon administration. For example, a large and relatively inert conjugate (i.e., having one or more high molecular weight PEG chains attached thereto, for example, one or more PEG chains having a molecular weight greater than about 10,000, wherein the conjugate possesses essentially no bioactivity) may be administered, which is hydrolyzed to generate a bioactive conjugate possessing a portion of the original PEG chain. In this way, the properties of the conjugate can be more effectively tailored to balance the bioactivity of the conjugate over time.

The water-soluble polymer associated with the conjugate can have a degradable linkage so as to provide a “cleavable” effect. That is, the water-soluble polymer cleaves (either through hydrolysis, enzymatic processes, or otherwise), thereby resulting in the unconjugated G-CSF moiety. In some instances, cleavable polymers detach from the G-CSF moiety in vivo without leaving any fragment of the water-soluble polymer. In other instances, cleavable polymers detach from the G-CSF moiety in vivo leaving a relatively small fragment (e.g., a succinate tag) from the water-soluble polymer. In both cases, the result is a conjugate that can provide a sustained release profile over time upon administration to a patient. An exemplary conjugate providing such a sustained release is one prepared with a polymer that is attached to the G-CSF moiety via a carbonate linkage or urethane linkage.

In those instances where a degradable linkage is a cleavable type of degradable linkage, the conjugates of the invention can be thought of as prodrugs (although the conjugate may retain activity even in the conjugate form). Exemplary degradable and cleavable linkages include carboxylate ester, phosphate ester, thiolester, anhydrides, acetals, ketals, acyloxyalkyl ether, imines, orthoesters, peptides and oligonucleotides. Such linkages can be readily prepared by appropriate modification of either the G-CSF moiety (e.g., the carboxyl group C terminus of the protein or a side chain hydroxyl group of an amino acid such as serine or threonine contained within the protein) and/or the polymeric reagent using coupling methods commonly employed in the art. Most preferred, however, are hydrolyzable linkages that are readily formed by reaction of a suitably activated polymer with a non-modified functional group contained within the moiety having G-CSF activity.

Alternatively, a hydrolytically stable linkage, such as an amide, urethane (also known as carbamate), amine, thioether (also known as sulfide), or urea (also known as carbamide) linkage can also be employed as the linkage for coupling the G-CSF moiety. Again, a preferred hydrolytically stable linkage is an amide. In one approach, a water-soluble polymer bearing an activated ester can be reacted with an amine group on the G-CSF moiety to thereby result in an amide linkage. In some embodiments, it is preferred that the linkage (and therefore the corresponding conjugate) lacks a 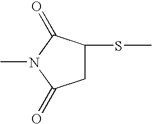
moiety. In some embodiments, it is preferred that the linkage (and therefore the corresponding conjugate) lacks the linkage produced by reaction of a phenyl glyoxal-terminated polymeric reagent and the G-CSF moiety. In some embodiments, it is preferred that the linkage lacks the linkage produced by reaction of a haloacetamide-terminated polymeric reagent and the G-CSF moiety.

The conjugates (as opposed to an unconjugated G-CSF moiety) may or may not possess a measurable degree of G-CSF activity. That is to say, a polymer-G-CSF moiety conjugate in accordance with the invention will possesses anywhere from about 0.1% to about 100% of the bioactivity of the unmodified parent G-CSF moiety. In some instances, the polymer-G-CSF moiety conjugates may posses greater than 100% bioactivity of the unmodified parent G-CSF moiety. Preferably, conjugates possessing little or no G-CSF activity contain a hydrolyzable linkage connecting the polymer to the moiety, so that regardless of the lack (or relative lack) of activity in the conjugate, the active parent molecule (or a derivative thereof) is released upon aqueous-induced degradation of the hydrolyzable linkage. Such activity may be determined using a suitable in-vivo or in-vitro model, depending upon the known activity of the particular moiety having G-CSF activity employed.

For conjugates possessing a hydrolytically stable linkage that couples the moiety having G-CSF activity to the polymer, the conjugate will typically possess a measurable degree of bioactivity. For instance, such conjugates are typically characterized as having a bioactivity satisfying one or more of the following percentages relative to that of the unconjugated G-CSF moiety: at least about 2%, at least about 5%, at least about 10%, at least about 15%, at least about 25%, at least about 30%, at least about 40%, at least about 50%, at least about 60%, at least about 80%, at least about 85%, at least about 90%, at least about 95%, at least about 97%, at least about 100%, and more than 105% (when measured in a suitable model, such as those well known in the art). Preferably, conjugates having a hydrolytically stable linkage (e.g., an amide linkage) will possess at least some degree of the bioactivity of the unmodified parent moiety having G-CSF activity.

Those of ordinary skill in the art will recognize that the foregoing discussion concerning nonpeptidic and water-soluble polymers is by no means exhaustive and is merely illustrative, and that all polymeric materials having the qualities described above are contemplated. As used herein, the term “polymeric reagent” generally refers to an entire molecule, which can comprise a water-soluble polymer segment and a functional group.

As described above, a conjugate of the invention comprises a water-soluble polymer covalently attached to a G-CSF moiety. Typically, for any given conjugate, there will be one to three water-soluble polymers covalently attached to one or more moieties having G-CSF activity. In some instances, however, the conjugate may have 1, 2, 3, 4, 5, 6, 7, 8 or more water-soluble polymers individually attached to a G-CSF moiety.

Exemplary conjugates in accordance with the invention will now be described. In describing the conjugates, references may be made to certain amino acids. Such references refer to the human G-CSF as provided in SEQ ID NO: 1 and are for convenience only. One having ordinary skill in the art will be able to readily determine the corresponding location or atom in other moieties having G-CSF activity. In particular, the description provided herein for native human G-CSF is often applicable to fragments, deletion variants, substitution variants or addition variants of any of the foregoing.

As shown above, the particular linkage within the moiety having G-CSF activity and the polymer depends on a number of factors. Such factors include, for example, the particular linkage chemistry employed, the particular G-CSF moiety, the available functional groups within the G-CSF moiety (either for attachment to a polymer or conversion to a suitable attachment site), the presence of additional reactive functional groups within the G-CSF moiety, and the like.

Amino groups on G-CSF moieties provide a point of attachment between the G-CSF moiety and the water-soluble polymer. In one embodiment, the conjugate has one water-soluble conjugate attached at the N-terminal of the G-CSF moiety, in some instances, however, the composition will contain less than 50% of N-terminus monoPEGylated conjugates. In exemplary conjugates, the N-terminally conjugated G-CSF moiety does not contain a methionine residue as the terminal amino acid. Human G-CSF comprises four amine-containing lysine residues and one amino terminus (see SEQ ID NO: 1). Thus, exemplary attachment points of this G-CSF include attachment at the amine side chain associated with a lysine at any one of positions 16, 23, 34 and 40.

There are a number of examples of suitable polymeric reagents useful for forming covalent linkages with available amines of a G-CSF moiety. Specific examples, along with the corresponding conjugate, are provided in Table 1, below. In the table, the variable (n) represents the number of repeating monomeric units and “—NH-(G-CSF)” represents the residue of the G-CSF moiety following conjugation to the polymeric reagent. While each polymeric portion [e.g., (OCH₂CH₂)ₙ or (CH₂CH₂O)ₙ] presented in Table 1 terminates in a “CH₃” group, other groups (such as H and benzyl) can be substituted therefor.

TABLE 1
Amine-Specific Polymeric Reagents and the G-CSF Moiety Conjugate Formed Therefrom
Polymeric Reagent

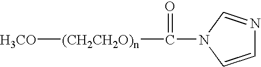

mPEG-Oxycarbonylimidazole Reagent

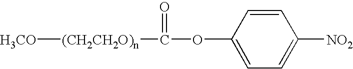

mPEG Nitrophenyl Reagent

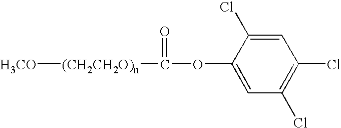

mPEG-Trichlorophenyl Carbonate Reagent

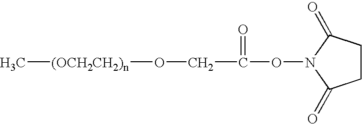

mPEG-Succinimidyl Reagent

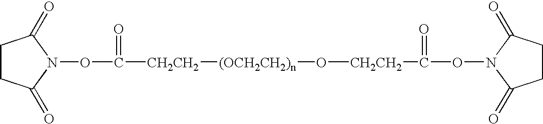

Homobifunctional PEG-Succinimidyl Reagent

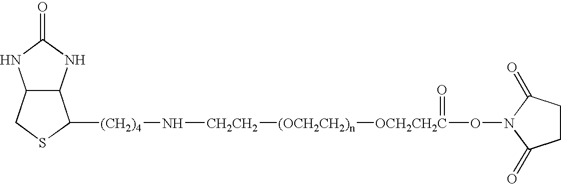

Heterobifunctional PEG-Succinimidyl Reagent

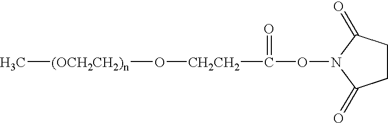

mPEG-Succinimidyl Reagent

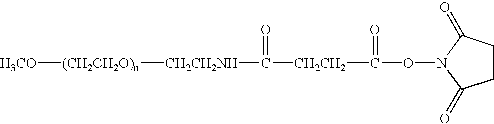

mPEG-Succinimidyl Reagent

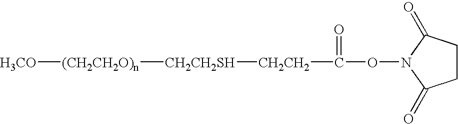

mPEG Succinimidyl Reagent

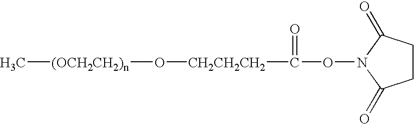

mPEG-Succinimidyl Reagent

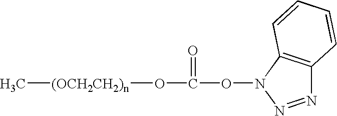

mPEG-Benzotriazole Carbonate Reagent

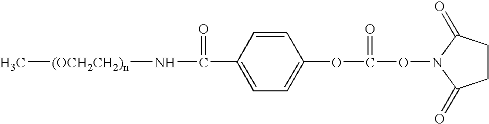

mPEG-Succinimidyl Reagent

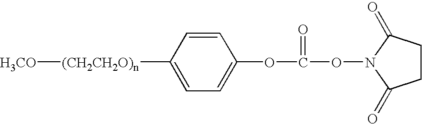

mPEG-Succinimidyl Reagent

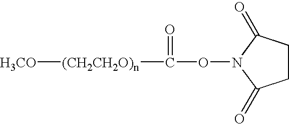

mPEG Succinimidyl Reagent

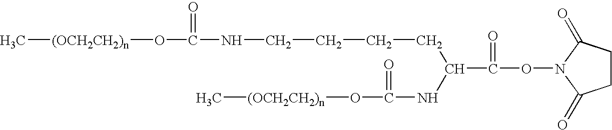

Branched mPEG2-N-Hydroxysuccinimide Reagent

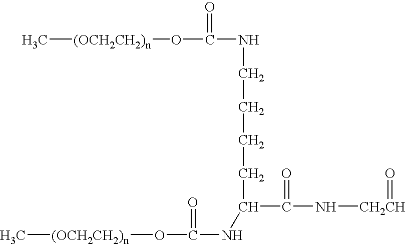

Branched mPEG2-Aldehyde Reagent

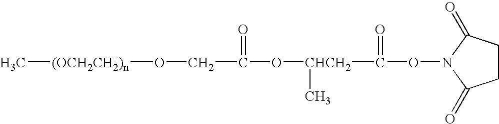

mPEG-Succinimidyl Reagent

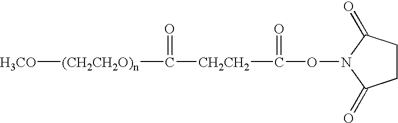

mPEG-Succinimidyl Reagent

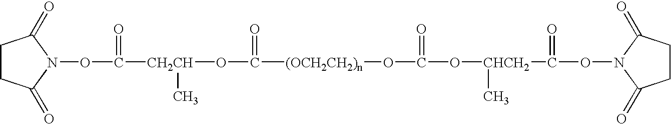

Homobifunctional PEG-Succinimidyl Reagent

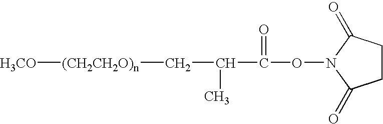

mPEG-Succinimidyl Reagent

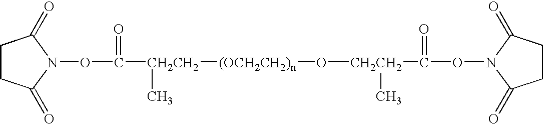

Homobifunctional PEG-Succinimidyl Propionate Reagent

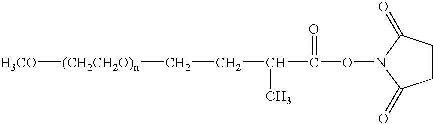

mPEG-Succinimidyl Reagent

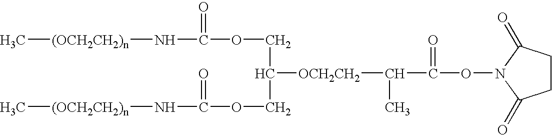

Branched mPEG2-N-Hydroxysuccinimide Reagent

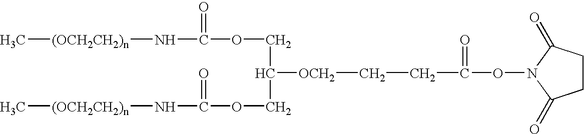

Branched mPEG2-N-Hydroxysuccinimide Reagent

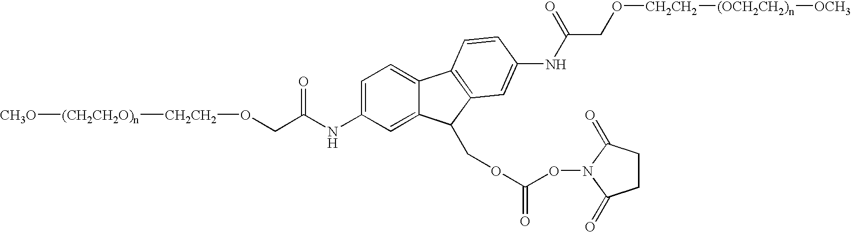

“Fulvene-based” Branched mPEG2-N-Hydroxysuccinimide Reagent

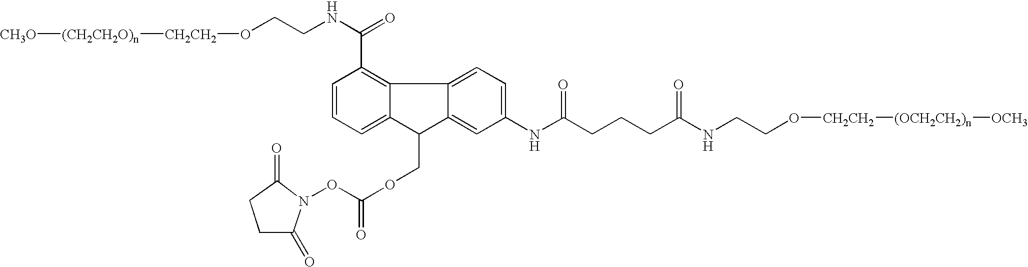

“Fulvene-based” Branched mPEG2-N-Hydroxysuccinimide Reagent

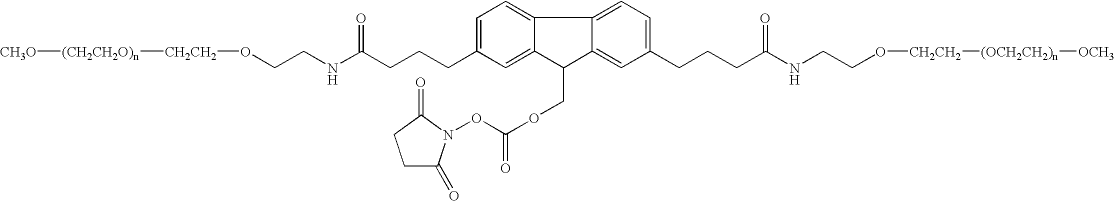

“Fulvene-based” Branched mPEG2-N-Hydroxysuccinimide Reagent

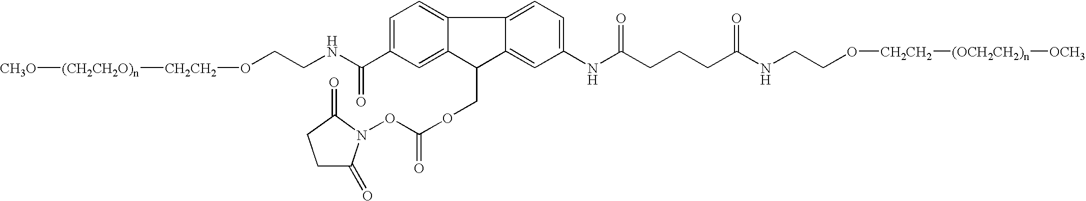

“Fulvene-based” Branched mPEG2-N-Hydroxysuccinimide Reagent

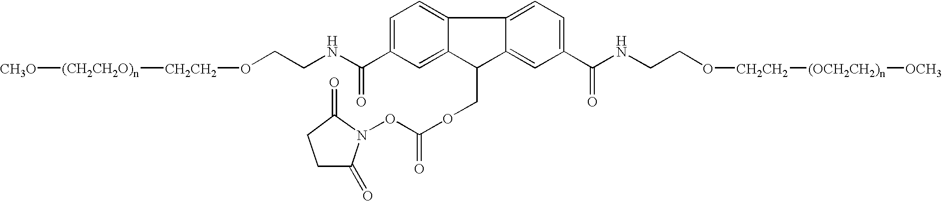

“Fulvene-based” Branched mPEG2-N-Hydroxysuccinimide Reagent

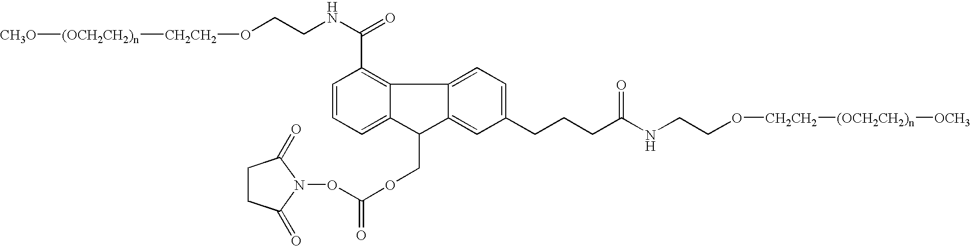

“Fulvene-based” Branched mPEG2-N-Hydroxysuccinimide Reagent

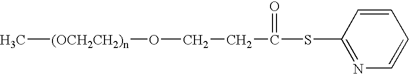

mPEG-Thioester Reagent

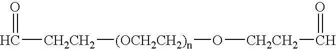

Homobifunctional PEG Propionaldehyde Reagent

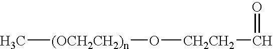

mPEG Propionaldehyde Reagent

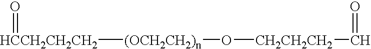

Homobifunctional PEG Butyraldehyde Reagent

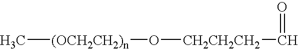

mPEG Butyraldehyde Reagent

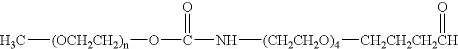

mPEG Butyraldehyde Reagent

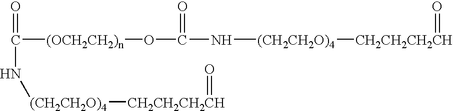

Homobifunctional PEG Butyraldehyde Reagent

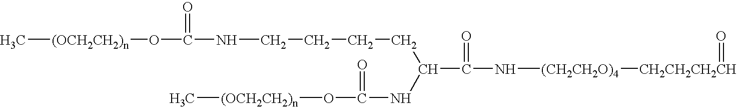

Branched mPEG2 Butyraldehyde Reagent

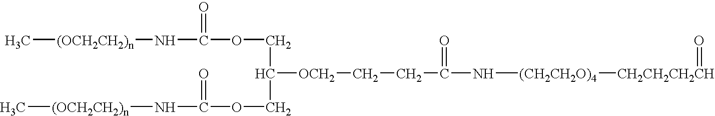

Branched mPEG2 Butyraldehyde Reagent

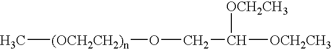

mPEG Acetal Reagent

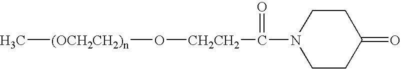

mPEG Piperidone Reagent

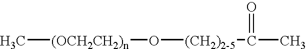

mPEG Methylketone Reagent

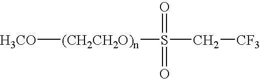

mPEG tresylate Reagent

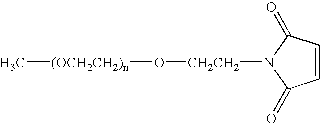

mPEG Maleimide Reagent (under certain reaction conditions such as pH >8)

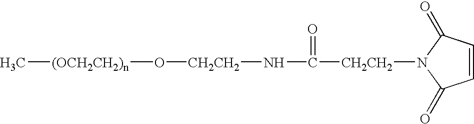

mPEG Maleimide Reagent (under certain reaction conditions such as pH >8)

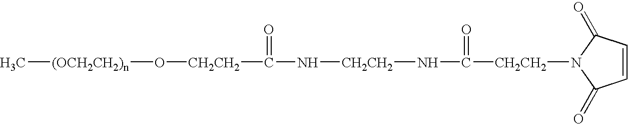

mPEG Maleimide Reagent (under certain reaction conditions such as pH >8)

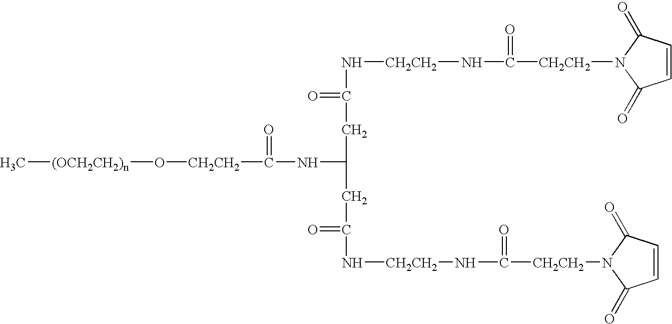

mPEG Forked Maleimide Reagent (under certain reaction conditions such as pH >8)

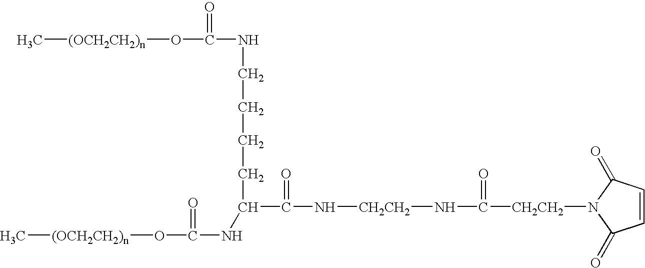

branched mPEG2 Maleimide Reagent (under certain reaction conditions such as pH >8)
Corresponding Conjugate

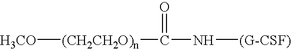

Carbamate Linkage

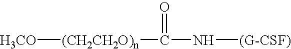

Carbamate Linkage

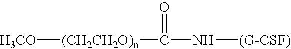

Carbamate Linkage

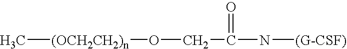

Amide Linkage

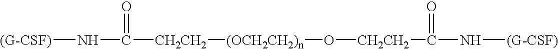

Amide Linkages

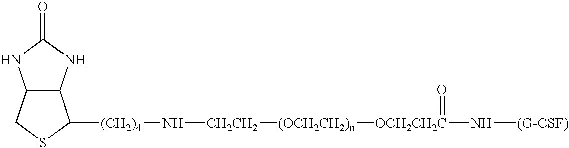

Amide Linkage

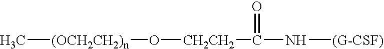

Amide Linkage

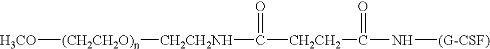

Amide Linkage

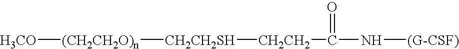

Amide Linkage

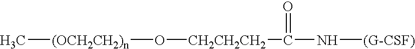

Amide Linkage

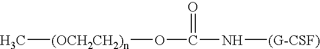

Carbamate Linkage

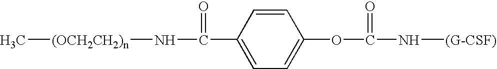

Carbamate Linkage

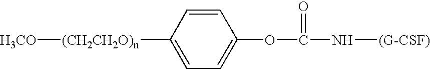

Amide Linkage

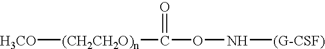

Amide Linkage

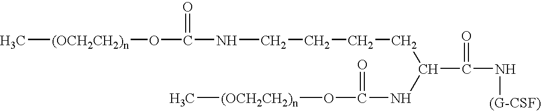

Amide Linkage

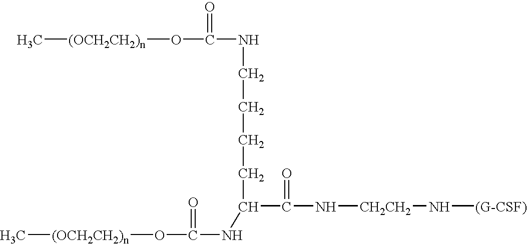

Secondary Amine Linkage

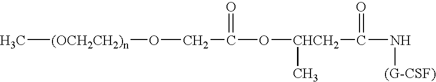

Amide Linkage

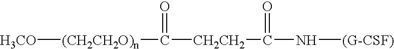

Amide Linkage

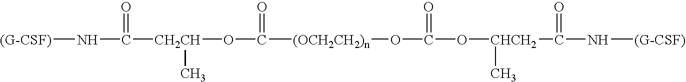

Amide Linkages

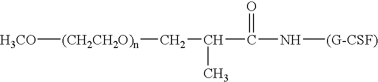

Amide Linkage

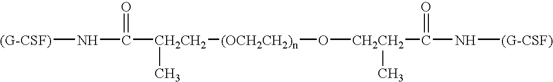

Amide Linkages

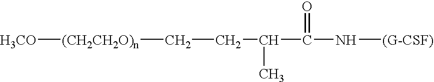

Amide Linkage

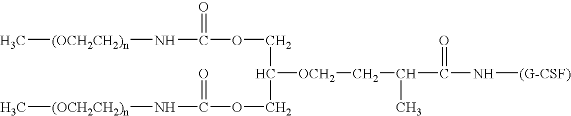

Amide Linkage

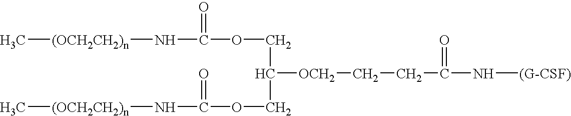

Amide Linkage

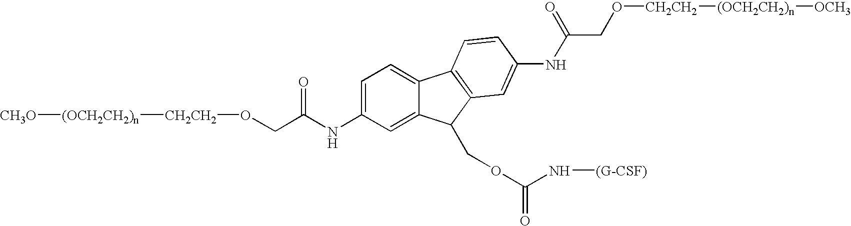

Amide Linkage

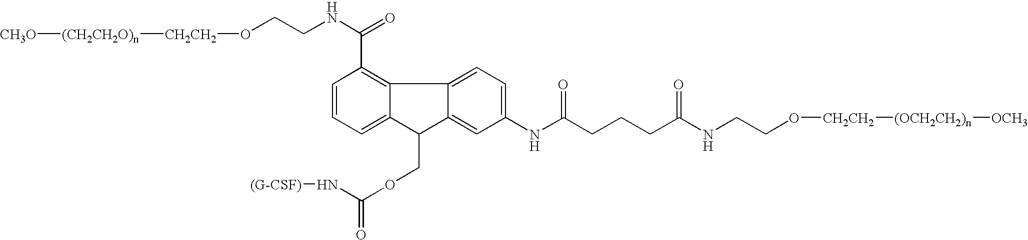

Amide Linkage

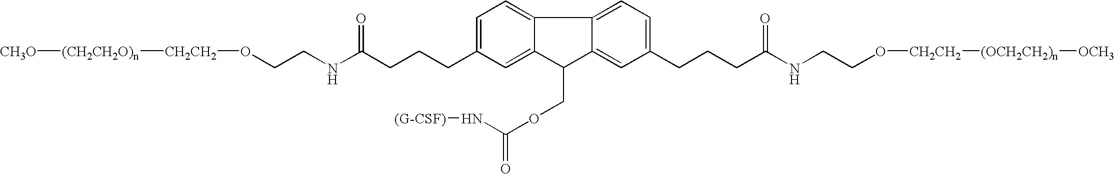

Amide Linkage

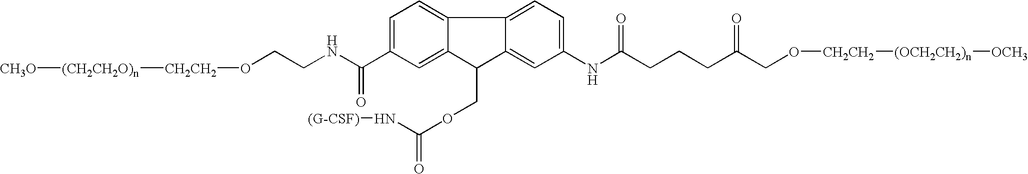

Amide Linkage

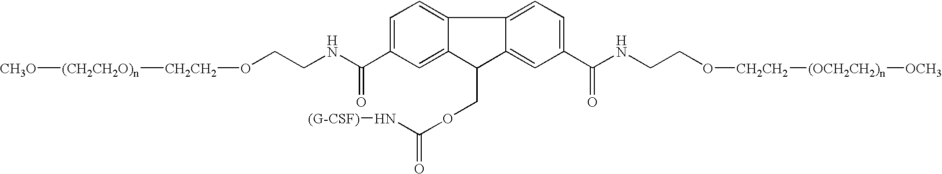

Amide Linkage

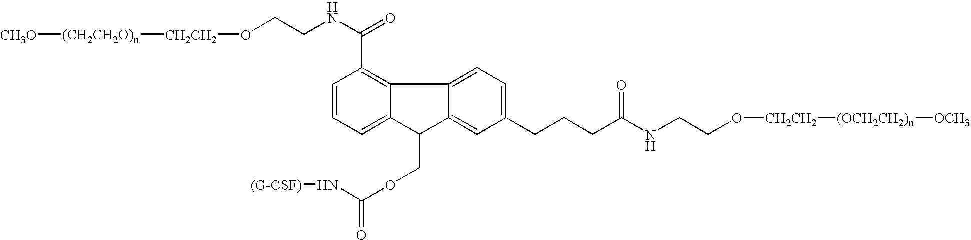

Amide Linkage

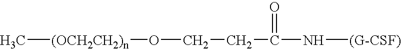

Amide Linkage (typically to G-CSF moiety having an N-terminal cysteine or histidine)

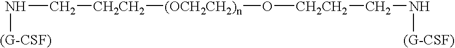

Secondary Amine Linkages

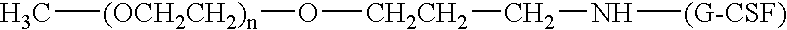

Secondary Amine Linkage

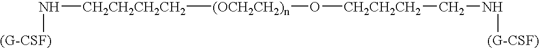

Secondary Amine Linkages

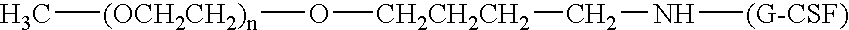

Secondary Amine Linkage

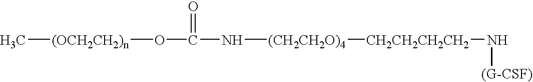

Secondary Amine Linkage

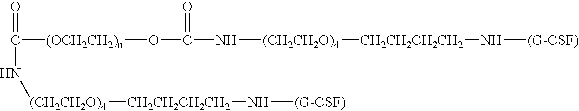

Secondary Amine Linkages

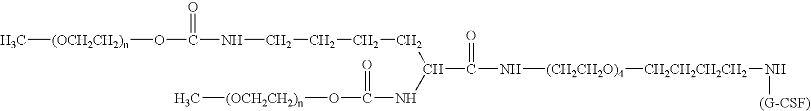

Secondary Amine Linkage

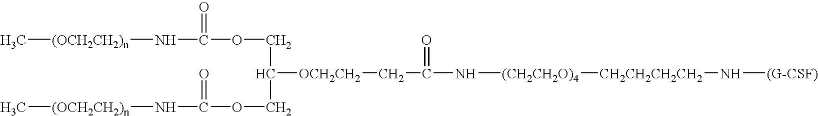

Secondary Amine Linkage

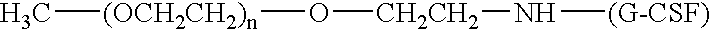

Secondary Amine Linkage

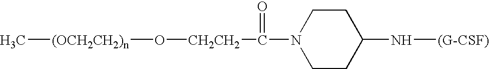

Secondary Amine Linkage (to a secondary carbon)

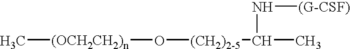

Secondary Amine Linkage (to a secondary carbon)

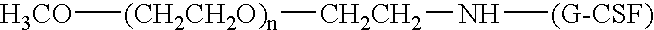

Secondary Amine Linkage

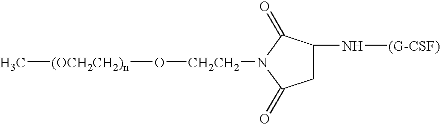

Secondary Amine Linkage

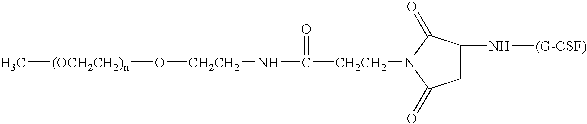

Secondary Amine Linkage

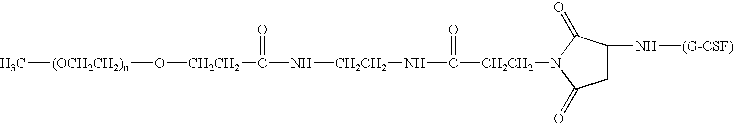

Secondary Amine Linkage

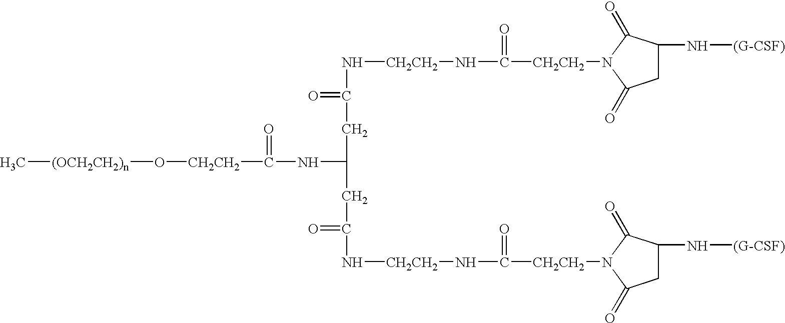

Secondary Amine Linkages

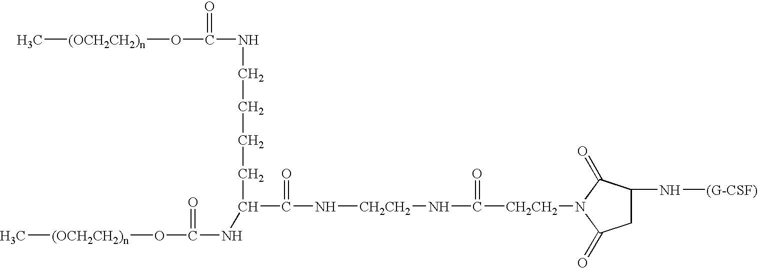

Secondary Amine Linkage

Conjugation of a polymeric reagent to an amino group of a G-CSF moiety can be accomplished by a variety of techniques. In one approach, a G-CSF moiety can be conjugated to a polymeric reagent functionalized with a succinimidyl derivative (or other activated ester group) In this approach, the polymer bearing a succinimidyl group (or other activated ester group) can be attached to the G-CSF moiety in an aqueous media at a pH of 7 to 9.0, although using different reaction conditions (e.g., a lower pH such as 6 to 7, or different temperatures and/or less than 15° C.) can result in the attachment of the polymer to a different location on the G-CSF moiety. In addition, an amide linkage can be formed by reacting an amine-terminated nonpeptidic, water-soluble polymer with a G-CSF moiety bearing an activating a carboxylic acid group.

An exemplary conjugate of the invention comprises a residue of a G-CSF moiety attached through an amide or a secondary amine linkage to a branched water-soluble polymer, wherein (i) an optional spacer moiety comprised of one or more atoms is located between the amide or secondary amine linkage and the branched water-soluble polymer, and (ii) the branched water-soluble polymer does not contain a lysine residue.

In addition, with respect to N-terminally modified conjugates, an exemplary composition comprises a plurality of conjugates, each conjugate comprising a residue of a G-CSF moiety attached, either directly or through a spacer moiety comprised of one or more atoms, to a water-soluble polymer, wherein less than 50% of all conjugates in the composition are not N-terminally monoPEGylated.

Exemplary conjugates in accordance with the invention have the following structure 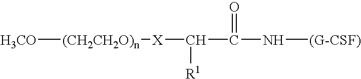
wherein:
(n) is an integer having a value of from 3 to 4000;
X is a spacer moiety comprised of one or more atoms;
R¹ is an organic radical containing 1 to 3 carbon atoms selected from the group consisting of methyl, ethyl, propyl, and isopropyl; and
G-CSF is a residue of a G-CSF moiety.

Exemplary conjugates of the present invention have the following structure: 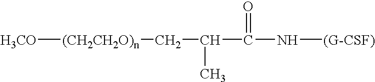
wherein (n) is an integer having a value of from 3 to 4000 and G-CSF is a residue of a G-CSF moeity.

Typical of another approach useful for conjugating the G-CSF moiety to a polymeric reagent is use of a reductive amination to conjugate a primary amine of a G-CSF moiety with a polymeric reagent functionalized with a ketone, aldehyde or a hydrated form thereof (e.g., a ketone hydrate or aldehyde hydrate). In this approach, the primary amine from the G-CSF moiety reacts with the carbonyl group of the aldehyde or ketone (or the corresponding hydroxyl-containing group of a hydrated aldehyde or ketone), thereby forming a Schiff base. The Schiff base, in turn, can then be reductively converted to a stable conjugate through use of a reducing agent such as sodium borohydride. Selective reactions (e.g., at the N-terminus) are possible, particularly with a polymer functionalized with a ketone or an alpha-methyl branched aldehyde and/or under specific reaction conditions (e.g., reduced pH).

Exemplary conjugates of the invention wherein the water-soluble polymer is in a branched form, will have the branched form of the water-soluble polymer having the following structure 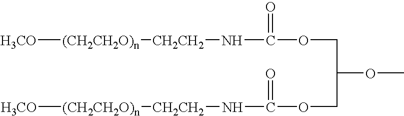
wherein each (n) is independently an integer having a value of from 3 to 4000.

Exemplary conjugates of the invention have the following structure: 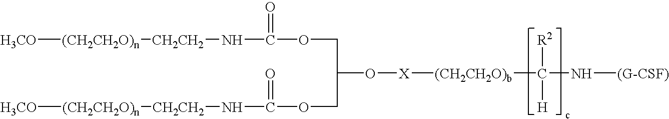
wherein:
each (n) is independently an integer having a value of from 3 to 4000;
X is a spacer moiety comprised of one or more atoms;
(b) is 2 through 6;
(c) is 2 through 6;
R², in each occurrence, is independently H or lower alkyl; and
G-CSF is a residue of a G-CSF moiety.

Exemplary conjugates of the invention have the following structure: 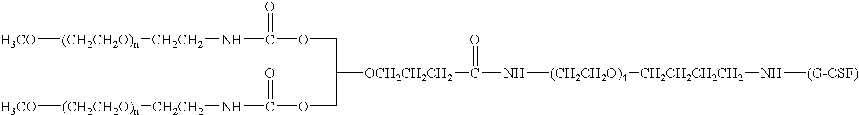
wherein:
each (n) is independently an integer having a value of from 3 to 4000; and
G-CSF is a residue of a G-CSF moiety.

Exemplary conjugates of the invention have the following structure: 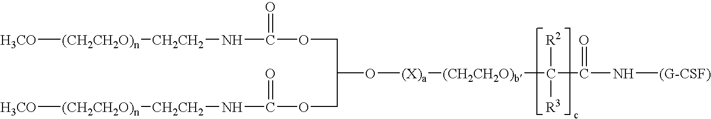
wherein:
each (n) is independently an integer having a value of from 3 to 4000;
(a) is either zero or one;
X, when present, is a spacer moiety comprised of one or more atoms;
(b′) is zero or an integer having a value of one through ten;
(c) is an integer having a value of one through ten;
R², in each occurrence, is independently H or an organic radical;
R³, in each occurrence, is independently H or an organic radical; and
G-CSF is a residue of a G-CSF moiety.

Exemplary conjugates of the invention have the following structure: 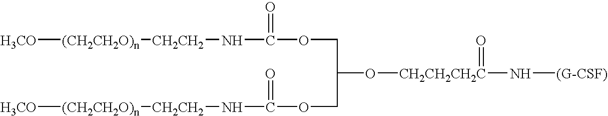
wherein:
each (n) is independently an integer having a value of from 3 to 4000; and
G-CSF is a residue of G-CSF moiety.

Exemplary conjugates of the invention have the following structure: 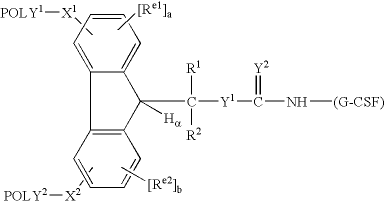
wherein:
POLY¹ is a first water-soluble polymer;
POLY² is a second water-soluble polymer;
X¹ is a first spacer moiety;
X² is a second spacer moiety;
H_{α} is an ionizable hydrogen atom;
R¹ is H or an organic radical;
R² is H or an organic radical;
(a) is either zero or one;
(b) is either zero or one;
R^{e1}, when present, is a first electron altering group;
R^{e2}, when present, is a second electron altering group;
Y¹ is O or S;
Y₂ is O or S; and
G-CSF is a residue of a G-CSF moiety.
   These conjugates (which are “fulvene based”) include a cleavable linkage wherein a G-CSF moiety is released in vivo upon following administration. Advantageously, such “fulvene-based” conjugates include instances where only one water-soluble polymer is present (e.g., POLY² and X² are absent) and are formed where the corresponding polymeric reagent (described in the paragraph immediately below) lacks POLY² and X².

Such fulvene-based conjugates can be prepared by combining, under conjugation conjugations, a G-CSF moiety with a fulvene-based polymeric reagent of the following structure: 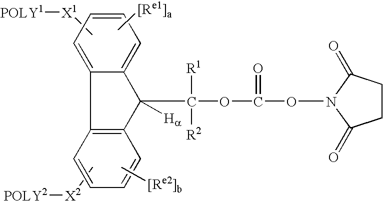
wherein:
POLY¹ is a first water-soluble polymer;
POLY² is a second water-soluble polymer;
X¹ is a first spacer moiety;
X² is a second spacer moiety;
H_{α} is an ionizable hydrogen atom;
R¹ is H or an organic radical;
R² is H or an organic radical;
(a) is either zero or one;
(b) is either zero or one;
R^{e1}, when present, is a first electron altering group; and
R^{e2}, when present, is a second electron altering group.

The synthesis of such fulvene-based polymeric reagents is described in co-owned and copending U.S. patent application Ser. No. 11/454,971. As described therein, fulvene-based polymeric reagents can be prepared in any number of ways. For example, one method for preparing a fulvene-based reagent comprises: (a) providing an aromatic-containing moiety bearing a first attachment site, a second attachment site and an optional third attachment site; (b) reacting a functional group reagent with the first attachment site to result in the first attachment site bearing a functional group capable of reacting with an amino group of an active agent and result in a degradable linkage, such as a carbamate; and (c) reacting a water-soluble polymer bearing a reactive group with the second attachment site and, when present, the optional third attachment site to result in (i) the second attachment site bearing a water-soluble polymer through a spacer moiety, and (ii) the optional third attachment site, when present, bearing a second water-soluble polymer through a spacer moiety. In some instances, (b) is performed before step (c) while in other instances, (c) is performed before step (b).

Thus, in this method for preparing a fulvene-based polymeric reagent, a required step is (a) providing an aromatic-containing moiety bearing a first attachment site, a second attachment site and an optional third attachment site. In the context of a synthetic preparation, it is understood that “providing” a material means to obtain the material (by, for example, synthesizing it or obtaining it commercially). An exemplary aromatic-containing moiety, for illustrative purposes, is 9-hydroxymethyl-2,7-diaminofluorene, as shown below.

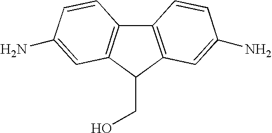

This aromatic-containing moiety, 9-hydroxymethyl-2,7-diaminofluorene, is an example of an aromatic-containing moiety having three attachment sites: a hydroxyl group at the 9 position and amino groups at each of the 2 and 7 positions. The aromatic-containing moiety can be provided in a base or salt form. With respect to 9-hydroxymethyl-2,7-diaminofluorene, it is possible to use the dihydrochloride form.

Having provided the aromatic-containing moiety, another step in the method for providing a fulvene-based polymeric reagent broadly includes the step of reacting a water-soluble polymer bearing a reactive group with the attachment site(s) on the aromatic-containing moiety. Here, any art-known approach for attaching a water-soluble polymer to one or more attachment sites on the aromatic-containing moiety can be used and the method is not limited to the specific approach. For example, an amine reactive PEG (such as an N-succinimidyl ester-terminated MPEG, formed, for example, from the reaction of N-hydroxysuccinimide and CH₃O—CH₂CH₂—(OCH₂CH₂)—OCH₂CH₂—OCH₂COOH with dicyclohexyl carbodiimide (DCC) or diisopropyl carbodiimide (DIC) as condensing agent and optionally in the presence of a base) can be reacted with amine bearing aromatic-containing moiety such as 9-hydroxymethyl-2,7-diaminofluorene.

In some instances, reaction of the water-soluble polymer bearing a reactive group with the aromatic-containing moiety will result in all possible attachment sites having water-soluble polymer attached thereto. In such circumstances it is necessary to remove at least one water-soluble polymer so that an attachment site is made available for reaction with a functional group reagent. Thus, for example, reaction of the N-succinimidyl ester-terminated mPEG discussed in the previous paragraph with 9-hydroxymethyl-2,7-diaminofluorene results in a mixture comprising (a) a species bearing two water-soluble polymers, one at each of the two amine sites, and (b) a species bearing three water-soluble polymers, one at each of the two amine sites, and one at the hydroxyl site. Here, it is possible to remove and collect higher molecular weight species by using size-exclusion chromatography. In addition it is possible to treat the mixture to high pH [treating, for example, the mixture to lithium hydroxide (LiOH), sodium hydroxide (NaOH), potassium hydroxide (KOH)], followed by ion-exchange chromatography (IEC). In either case, the result is a composition containing mostly 9-hydroxymethyl-2,7-diaminofluorene bearing two water-soluble polymers, one at each of the two amine sites. A third hydroxyl site is thereby available for reaction with a functional group reagent.

The final step is reacting a reactive site of the aromatic-containing moiety with a functional group reagent. A preferred approach is to react the hydroxyl-containing 9-hydroxymethyl-2,7-diaminofluorene bearing two water-soluble polymers, one at each of the two amine sites with triphosgene followed by treatment with N-hydroxysuccinimide. In this way, a functional group capable of reacting with an amino group of an active agent to form a degradable linkage, such as a carbamate linkage (in this case, an “activated carbonate”) is formed on the hydroxyl-containing reactive site.

The steps of the method for providing the fulvene-based polymeric reagents take place in an appropriate solvent. One of ordinary skill in the art can determine whether any specific solvent is appropriate for any given reaction. Typically, however, the solvent is preferably a nonpolar solvent or a polar aprotic solvent. Nonlimiting examples of nonpolar solvents include benzene, xylene, dioxane, tetrahydrofuran (THF), t-butyl alcohol and toluene. Particularly preferred nonpolar solvents include toluene, xylene, dioxane, tetrahydrofuran, and t-butyl alcohol. Exemplary polar aprotic solvents include, but are not limited to, DMSO (dimethyl sulfoxide), HMPA (hexamethylphosphoramide), DMF (dimethylformamide), DMA (dimethylacetamide), NMP (N-methylpyrrolidinone).

Preferred amine groups in G-CSF that can serve as a site for attaching a polymer include those amine groups found within a lysine residue, such as Lys 16, Lys 34 and Lys 40. In addition, the N-terminus of any G-CSF moiety that is a protein can serve as a polymeric attachment site.

Carboxyl groups represent another functional group that can serve as a point of attachment on the G-CSF moiety. Structurally, the conjugate will comprise the following: 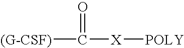
where (G-CSF) and the adjacent carbonyl group corresponds to the carboxyl-containing G-CSF moiety, X is a spacer moiety, preferably in this case a heteroatom selected from O, N(H), and S, and POLY is a water-soluble polymer such as PEG, optionally terminating in an end-capping moiety.

The C(O)-X linkage results from the reaction between a polymeric derivative bearing a terminal functional group and a carboxyl-containing G-CSF moiety. As discussed above, the specific linkage will depend on the type of functional group utilized. If the polymer is end-functionalized or “activated” with a hydroxyl group, the resulting linkage will be a carboxylic acid ester and X will be O. If the polymer backbone is functionalized with a thiol group, the resulting linkage will be a thioester and X will be S. When certain multi-arm, branched or forked polymers are employed, the C(O)X moiety, and in particular the X moiety, may be relatively more complex and may include a longer linkage structure.

Water-soluble derivatives containing a hydrazide moiety are also useful for conjugation at a carbonyl. To the extent that the G-CSF moiety does not contain a carbonyl moiety, a carbonyl moiety can be introduced by reducing any carboxylic acids (e.g., the C-terminal carboxylic acid) and/or by providing glycosylated or glycated (wherein the added sugars have a carbonyl moiety) versions of the G-CSF moiety. Specific examples of water-soluble derivatives containing a hydrazide moiety, along with the corresponding conjugates, are provided in Table 2, below. In addition, any water-soluble derivative containing an activated ester (e.g., a succinimidyl group) can be converted to contain a hydrazide moiety by reacting the water-soluble polymer derivative containing the activated ester with hydrazine (NH₂—NH₂) or tert-butyl carbazate [NH₂NHCO₂C(CH₃)₃]. In the table, the variable (n) represents the number of repeating monomeric units and “═C-(G-CSF)” represents the residue of the G-CSF moiety following conjugation to the polymeric reagent. Optionally, the hydrazone linkage can be reduced using a suitable reducing agent. While each polymeric portion [e.g., (OCH₂CH₂)ₙ or (CH₂CH₂O)ₙ] presented in Table 1 terminates in a “CH₃” group, other groups (such as H and benzyl) can be substituted therefor.

TABLE 2
Carboxyl-Specific Polymeric Reagents and the G-CSF Moiety Conjugate Formed Therefrom
Polymeric Reagent

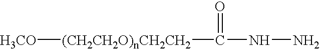

mPEG-Hydrazine Reagent

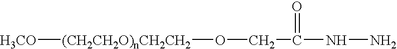

mPEG-Hydrazine Reagent

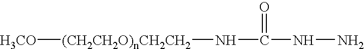

mPEG-Hydrazine Reagent

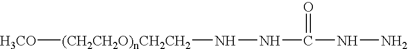

mPEG-Hydrazine Reagent

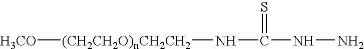

mPEG-Hydrazine Reagent

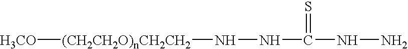

mPEG-Hydrazine Reagent

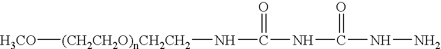

mPEG-Hydrazine Reagent

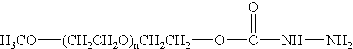

mPEG-Hydrazine Reagent
Corresponding Conjugate

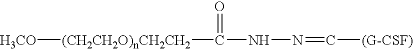

Hydrazone Linkage

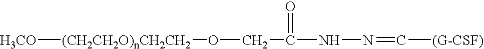

Hydrazone Linkage

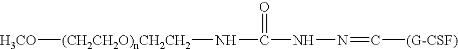

Hydrazone Linkage

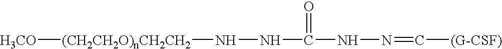

Hydrazone Linkage

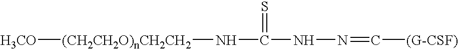

Hydrazone Linkage

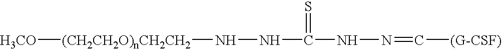

Hydrazone Linkage

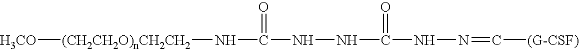

Hydrazone Linkage

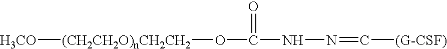

Hydrazone Linkage

Thiol groups contained within the G-CSF moiety can serve as effective sites of attachment for the water-soluble polymer. In particular, cysteine residues in the G-CSF moiety provide thiol groups when the G-CSF moiety is a protein. The thiol groups in such cysteine residues can then be reacted with an activated PEG that is specific for reaction with thiol groups, e.g., an N-maleimidyl polymer or other derivative, as described in U.S. Pat. No. 5,739,208 and in International Patent Publication No. WO 01/62827.

With respect to SEQ ID NOs: 1 through 3, there are five thiol-containing cysteine residues. Thus, preferred thiol attachment sites are associated with one of these five cysteine residues. Although it is preferred not to disrupt any disulfide bonds, it may be possible to attach a polymer within the side chain of one or more of these cysteine residues and retain a degree of activity. To the extent that any particular G-CSF moiety lacks a thiol group or disruption of disulfide bonds is to be avoided, however, it is possible to add a cysteine residue to the G-CSF moiety using conventional synthetic techniques. See, for example, the procedure described in WO 90/12874 for adding cysteine residues, wherein this procedure can be adapted for a G-CSF moiety. In addition, conventional genetic engineering processes can also be used to introduce a cysteine residue into the G-CSF moiety. In some embodiments, however, it is preferred not to introduce and additional cysteine residue and/or thiol group.

Specific examples, along with the corresponding conjugate, are provided in Table 3, below. In the table, the variable (n) represents the number of repeating monomeric units and “—S-(G-CSF)” represents the G-CSF moiety residue following conjugation to the water-soluble polymer. While each polymeric portion [e.g., (OCH₂CH₂)ₙ or (CH₂CH₂O)ₙ] presented in Table 3 terminates in a “CH₃” group, other groups (such as H and benzyl) can be substituted therefor.

TABLE 3
Thiol-Specific Polymeric Reagents and the G-CSF Moiety Conjugate Formed Therefrom
Polymeric Reagent

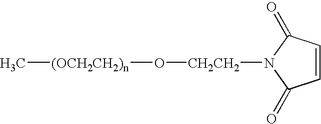

mPEG Maleimide Reagent

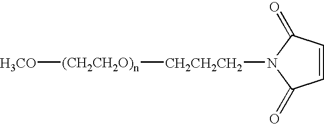

mPEG Maleimide Reagent

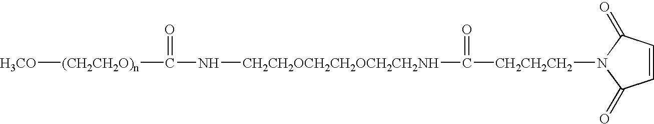

mPEG Maleimide Reagent

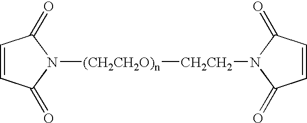

Homobifunctional mPEG Maleimide Reagent

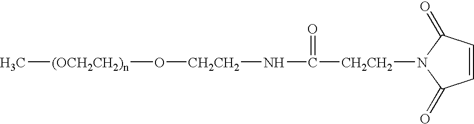

mPEG Maleimide Reagent

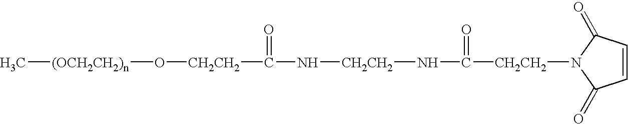

mPEG Maleimide Reagent

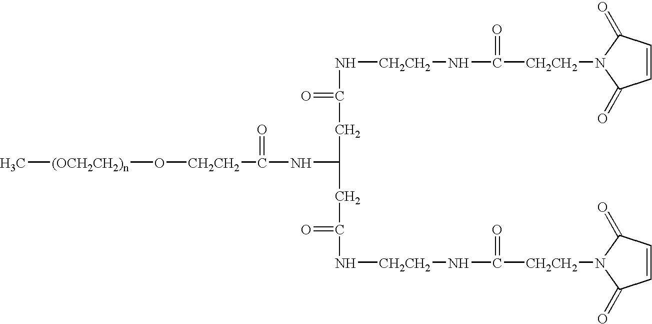

mPEG Forked Maleimide Reagent

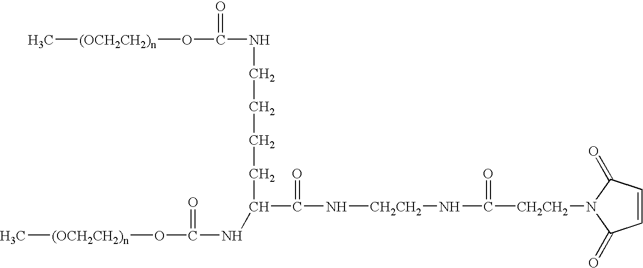

branched mPEG2 Maleimide Reagent

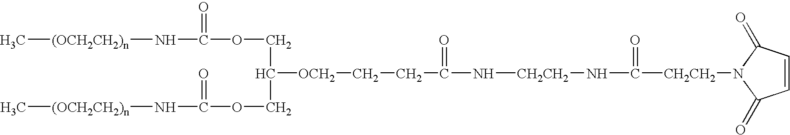

branched mPEG2 Maleimide Reagent

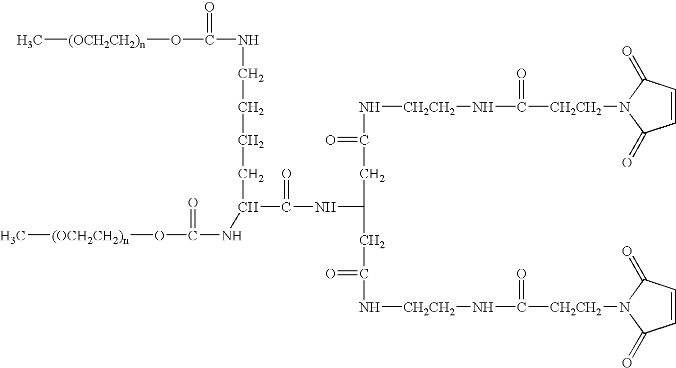

Branched mPEG2 Forked Maleimide Reagent

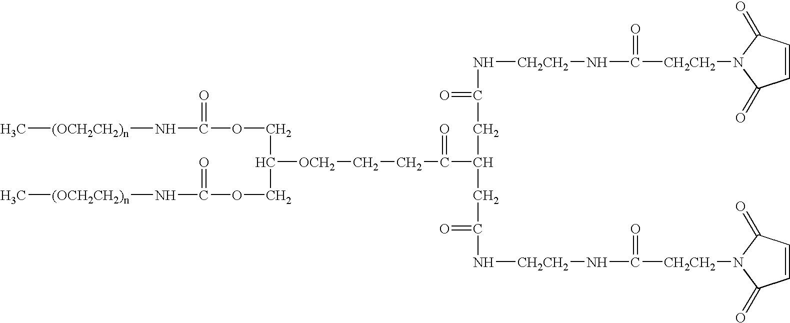

Branched mPEG2 Forked Maleimide Reagent

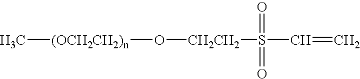

mPEG Vinyl Sulfone Reagent

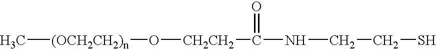

mPEG Thiol Reagent

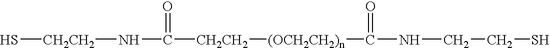

Homobifunctional PEG Thiol Reagent

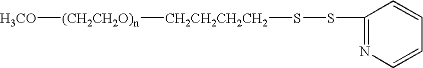

mPEG Disulfide Reagent

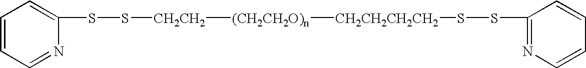

Homobifunctional Disulfide Reagent
Corresponding Conjugate

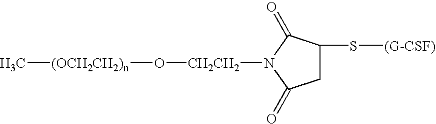

Thioether Linkage

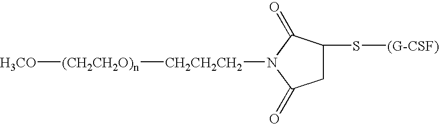

Thioether Linkage

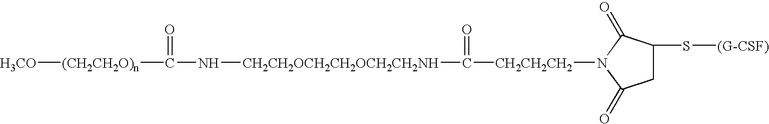

Thioether Linkage

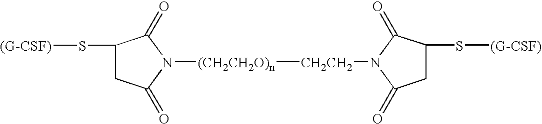

Thioether Linkage

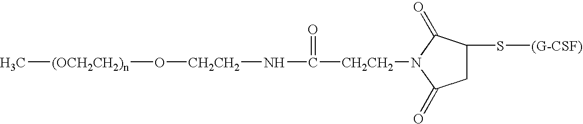

Thioether Linkage

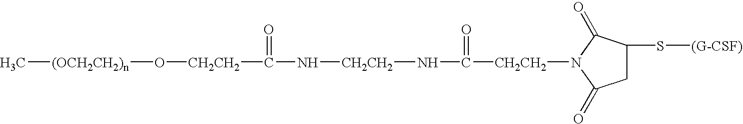

Thioether Linkage

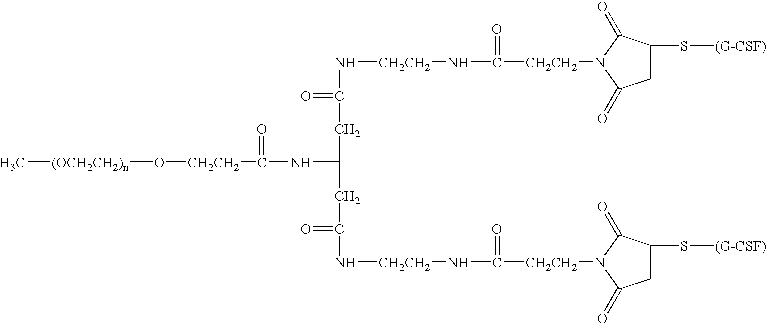

Thioether Linkage

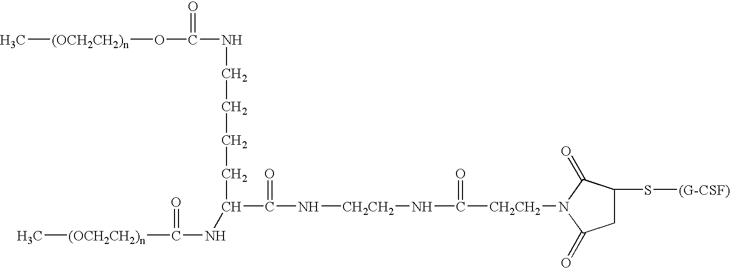

Thioether Linkage

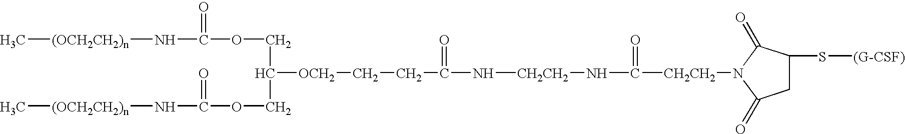

Thioether Linkage

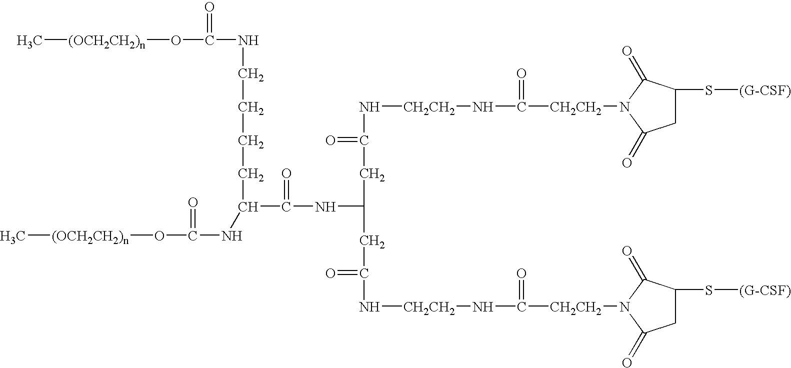

Thioether Linkages

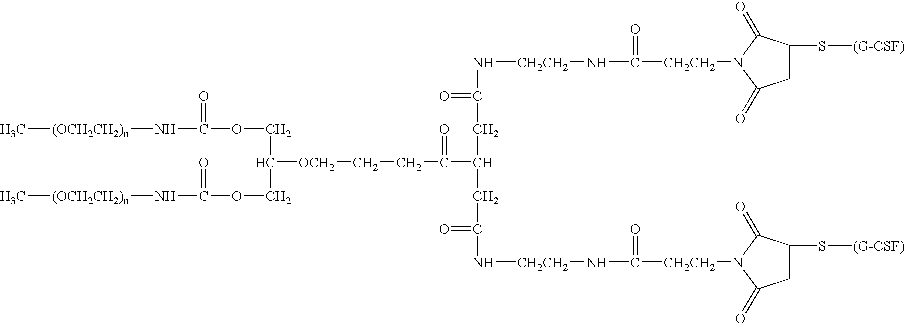

Thioether Linkages

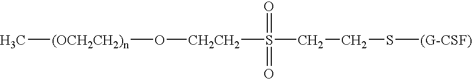

Thioether Linkage

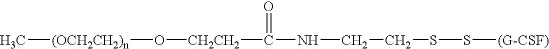

Disulfide Linkage

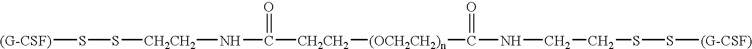

Disulfide Linkages

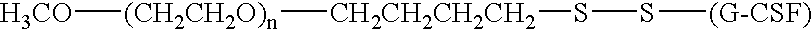

Disulfide Linkage

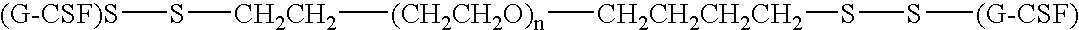

Disulfide Linkages

With respect to conjugates formed from water-soluble polymers bearing one or more maleimide functional groups (regardless of whether the maleimide reacts with an amine or thiol group on the G-CSF moiety), the corresponding maleamic acid form(s) of the water-soluble polymer can also react with the G-CSF moiety. Under certain conditions (e.g., a pH of about 7-9 and in the presence of water), the maleimide ring will “open” to form the corresponding maleamic acid. The maleamic acid, in turn, can react with an amine or thiol group of an G-CSF moiety. Exemplary maleamic acid-based reactions are schematically shown below. POLY represents the water-soluble polymer, and (G-CSF) represents the G-CSF moiety.

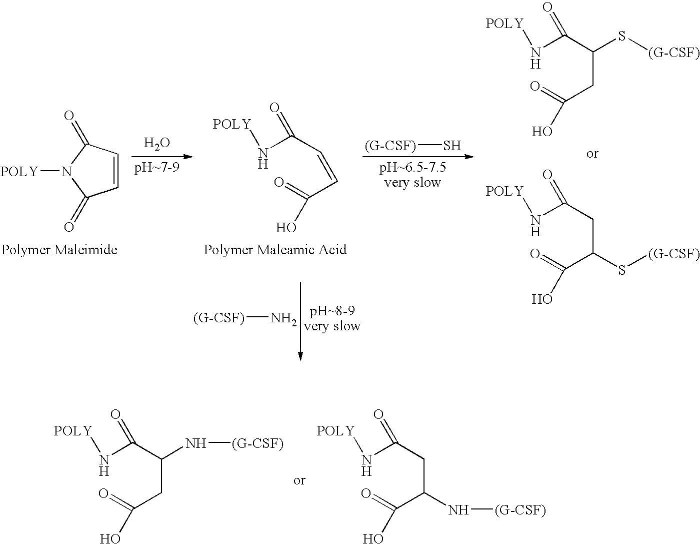

Polymeric reagents suited to be used to form G-CSF conjugates of the invention comprise the structure POLY- [OL-WN
POLY-[Y—S—W]ₓ
wherein:
POLY is a water-soluble polymer segment;
x is 1 to 25;
Y is a divalent linking group comprising at least four carbon atoms, and consisting of a saturated or unsaturated hydrocarbon backbone which is three to eight carbon atoms in length and has substituents which are independently selected from hydrogen, lower alkyl, lower alkenyl, and non-interfering substituents as defined herein, where two such alkyl and/or alkenyl substituents on different carbon atoms of the backbone may be linked so as to form a cycloalkyl, cycloalkenyl, or aryl group;
S is a sulfur atom attached to an sp³ hybridized carbon of Y;
and S—W is a thiol (i.e. W is H), protected thiol, or thiol-reactive derivative, such as ortho-pyridyl disulfide (OPSS). Protected thiols include, for example, thioethers, such as S-benzyl or S-trityl ethers, and thioesters. Such polymeric reagents are described in U.S. Patent Application Publication No. 2006/0135586.

A representative conjugate in accordance with the invention can have the following structure:
POLY-L_{0,1}-C(O)Z—Y—S—S-(G-CSF)
wherein POLY is a water-soluble polymer, L is an optional linker, Z is a heteroatom selected from the group consisting of O, NH, and S, and Y is selected from the group consisting of C₂₋₁₀ alkyl, C₂₋₁₀ substituted alkyl, aryl, and substituted aryl, and (G-CSF) is a residue of a G-CSF moiety. Polymeric reagents that can be reacted with a G-CSF moiety and result in this type of conjugate are described in U.S. Patent Application Publication No. 2005/0014903.

Conjugates can be formed using thiol-specific polymeric reagents in a number of ways and the invention is not limited in this regard. For example, the G-CSF moiety—optionally in a suitable buffer (including amine-containing buffers, if desired)—is placed in an aqueous media at a pH of about 7-8 and the thiol-specific polymeric reagent is added at a molar excess. The reaction is allowed to proceed for about 0.5 to 2 hours, although reaction times of greater than 2 hours (e.g., 5 hours, 10 hours, 12 hours, and 24 hours) can be useful if PEGylation yields are determined to be relatively low. Exemplary polymeric reagents that can be used in this approach are polymeric reagents bearing a reactive group selected from the group consisting of maleimide, sulfone (e.g., vinyl sulfone), and thiol (e.g., protected thiols such as an ortho pyridinyl or “OPSS”.

Preferred thiol groups in a G-CSF moiety that can serve as a site for attaching a polymeric reagent include those thiol groups found within cysteine residues. A particularly preferred thiol group is the thiol group associated with the side chain of the amino acid residue cysteine located at position 17.

Thus, an exemplary conjugate of the invention comprises a residue of a G-CSF moiety having a cysteine residue side chain corresponding to amino acid position 17 of hG-CSF, wherein the cysteine residue side chain is attached, either directly or through a spacer moiety comprised of one or more atoms, to a water-soluble polymer.

As previously described, PEGylation yields for thiol-based conjugation of some G-CSF moieties may be relatively low. Even allowing for extended reaction times, such PEGylation yields may still nevertheless be unsatisfactory. In these cases, it can still be possible to provide thiol-based modification in relatively large yields by employing a method for preparing a conjugate, the method comprising: (a) adding a first polymeric reagent composition (i.e., a composition comprising a first polymeric reagent) to a G-CSF moiety composition under conditions sufficient to result in a first conjugate composition (i.e., a composition comprising a first conjugate) comprising a first conjugate comprised of a G-CSF moiety covalently attached, either directly or through a first spacer moiety comprised of one or more atoms, to a first water-soluble polymer; and (b) adding a second polymeric reagent composition (i.e., a composition comprising a second polymeric reagent) to the first conjugate composition to result in a second conjugate composition (i.e., a composition comprising a second conjugate) comprising a second water-soluble polymer attached, either directly or through a second spacer moiety comprised of one or more atoms, to the first water-soluble polymer of the conjugate.

In accordance with the method, a polymeric reagent having a relatively small weight average molecular weight can be used for initial attachment to the G-CSF moiety. Thereafter, a polymeric reagent having a relatively large weight average molecular weight can be used. While not wishing to be bound by theory, it is believed that by using such an approach, the polymeric reagent having a relatively small weight average molecular weight can more completely react with a sterically hindered location within the G-CSF moiety than a relatively high weight average molecule weight polymeric reagent would. In this way, it is possible to more efficiently prepare the desired conjugates.

Thiol-based modification according to this method utilizes polymeric reagents bearing one or more functional groups that are capable of reacting with the thiol group-containing side chain of a cysteine residue. Such PEG reagents include, without limitation, PEG orthopyridyl disulfide reagents, PEG vinylsulfone reagents, PEG maleimide reagents, and PEG iodoacetimide reagents. These and other polymeric reagents are provided in Table 3.

The polymeric reagents used in accordance with this method can be hetereobifunctional or homobifunctional in nature.

The polymeric reagent having a relatively low weight average molecular weight will have a weight average molecular weight in the range of from about 100 Daltons to about 5,000 Daltons. Exemplary, weight average molecular weights in this range include: about 100 Daltons, about 150 Daltons, about 200 Daltons, about 250 Daltons, about 300 Daltons, about 300 Daltons, about 350 Daltons, about 400 Daltons, about 450 Daltons, about 500 Daltons, about 600 Daltons, about 700 Daltons, about 800 Daltons, about 900 Daltons, about 1000 Daltons, about 1,500 Daltons, about 2,000 Daltons, about 2,500 Daltons, about 3,000 Daltons, about 3,500 Daltons, about 4,000 Daltons, about 4,500 Daltons, and about 5,000 Daltons. An exemplary polymeric reagent having a relatively low weight average molecular weight has the following structure:
Y′—CH₂CH₂O(CH₂CH₂O)ₙCH₂CH₂—Y″ Formula I
wherein Y′ is an electrophilic or nucleophilic group and Y″ a reactive group suited to react with a functional group associated with the G-CSF moiety (e.g., Y″ can be a maleimide, sulfone or thiol for reaction with a thiol group associated with a G-CSF moiety, an aldehyde, ketone or succinimidyl for reaction with an amine group associated with a G-CSF moiety, and so forth), and (n) is an integer having a value from 2 to about 114, preferably having a value of from about 3 to about 6 (e.g., any one of 3, 4, 5 and 6).

The polymeric reagent having a relatively low weight average molecular weight can optionally be monodispersed (although monodispersity is not a requirement). By using a polymeric reagent that is monodispersed, it is possible to prepare compositions comprising conjugates comprised of one or more water-soluble polymers covalently attached to a G-CSF moiety, wherein each water-soluble polymer has (n) repeating monomers, and (ii) each (n) of the one or more water-soluble polymers covalently attached to the G-CSF moiety in every conjugate in the composition is the same.

The polymeric reagent having a relatively high weight average molecular weight will have a weight average molecular weight in the range of from about 100 Daltons to about 150,000 Daltons. Exemplary ranges, however, include weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. An exemplary polymeric reagent having a relatively high weight average molecular weight has the following structure:
Z′-CH₂CH₂O(CH₂CH₂O)ₙCH₂CH₂-Z″ (Formula II)
wherein Z″ is reactive to Y′ of the polymeric reagent having a relatively low weight average molecular weight (Formula I), Z′ is an end-capping group of a functional group, and (n′) is an integer having a value from 2 to about 3,400. With respect to the relatively high weight average molecular weight polymeric reagent, exemplary forms include linear and branched polymeric reagents.

A schematic for such an approach is provided below (wherein G-CSF represents a residue of a G-CSF moiety):

### Schematic for Preparing Conjugates at a Thiol moiety of a G-CSF Moiety

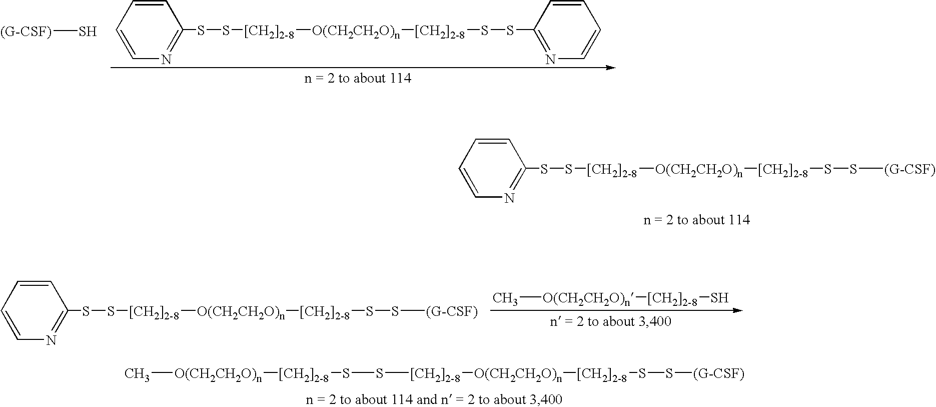

It will be recognized that the above schematic is for illustrative purposes only, and that (for example) other polymeric reagents can be used in accordance with the method. Thus, for example, polymeric reagents can be used in accordance with the above schematic to result in the following structure: 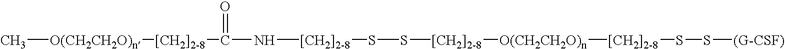
wherein (n) is an integer of from 2 to about 114, n′ is an integer from 2 to about 3,400, and G-CSF is a residue of a G-CSF moiety.

In an alternative method for attachment to an internal amino acid residue, such as a cysteine residue (e.g., cysteine 17), it is possible to conduct the PEGylation via a single step wherein the reactive group (e.g., a thiol reactive group such as maleimide) is optionally provided on a relatively long tethering group [e.g., an ethylene oxide polymer, a biocompatible polymer containing, for example, polymaminoacids (i.e., a polymer of the same or different amino acids), polycarbohydrates (i.e., a polymer of the same or different carbohydrates) as polymonosaccharides, polylacticacids, and so forth, and combinations of any of the foregoing]. Optionally, the polymer attached to the G-CSF moiety can, in turn, be attached to a second polymer (e.g., a branched polymer). Such reagents are described in the literature as well as in U.S. Pat. No. 6,774,180 and in U.S. patent application Ser. No. 10/734,858.

No matter which method is used, it is preferred to carry out the method for attaching a water-soluble polymer to the G-CSF moiety in a pH below 10, more preferably below a pH below 8.5, still more preferably below 8.25, yet still more preferably below 8.0, and most preferably below 7.5.

In those instances where a method that uses two polymeric reagents results, a conjugate having the following structure is formed:
POLY″-(X²)_{b}-POLY′-(X′)ₐ-(G-CSF)
wherein:
POLY″ is a second water-soluble polymer (preferably branched or straight);
POLY′ is a first water-soluble polymer or a biocompatible polymer
X¹, when present, is a first spacer moiety comprised of one or more atoms;
X², when present, is a second spacer moiety comprised of one or more atoms;
(b) is either zero or one;
(a) is either zero or one; and
G-CSF is a residue of a G-CSF moiety.

With respect to polymeric reagents, those described here and elsewhere can be purchased from commercial sources (e.g., Nektar Therapeutics, Huntsville, Ala.). In addition, methods for preparing the polymeric reagents are described in the literature.

The attachment between the G-CSF moiety and the nonpeptidic, water-soluble polymer (as well as other attachments between different parts of the conjugates described herein, such as attachment between two water-soluble polymers) can be direct, (e.g., wherein no intervening atoms are located between the G-CSF moiety and the polymer), or indirect, (e.g., wherein one or more atoms are located between the G-CSF moiety and the polymer). With respect to the indirect attachment, one or more atoms [conventionally referred to as a “spacer moiety,” (and identified as X¹, X², and so forth here) which can include one or more of carbon atoms, nitrogen atoms, sulfur atoms, oxygen atoms, and combinations thereof) is used to link adjacent atoms, thereby providing indirect attachment. The spacer moiety can comprise an amide, secondary amine, carbamate, thioether, or disulfide group. Nonlimiting examples of specific spacer moieties include those selected from the group consisting of —O—, —S—, —S—S—, —CH₂—S—S—CH₂—, —CH₂—CH₂—S—S—CH₂—CH₂—, —CH₂—CH₂—CH₂—S—S—CH₂—CH₂—CH₂—, —CH₂—CH₂—CH₂—CH₂—S—S—CH₂—CH₂—CH₂—CH₂—, —C(O)—NH—CH₂—CH₂—S—S—CH₂—CH₂—CH₂CH₂, —CH₂—CH₂—CH₂—CH₂—S—S—CH₂—CH₂—NH—C(O)—, —C(O)—, —C(O)—NH—, —NH—C(O)—NH—, —O—C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]ₕ—(OCH₂CH₂)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment. In some instances, it is preferred that the spacer moiety does not include two or more amino acid residues (e.g., the spacer moiety does not include -Gly-Gly-).

Compositions

The conjugates are typically part of a composition. Generally, the composition comprises a plurality of conjugates, preferably although not necessarily, each conjugate is comprised of the same G-CSF moiety (i.e., within the entire composition, only one type of G-CSF moiety is found). In addition, the composition can comprise a plurality of conjugates wherein any given conjugate is comprised of a moiety selected from the group consisting of two or more different G-CSF moieties (i.e., within the entire composition, two or more different G-CSF moieties are found). Optimally, however, substantially all conjugates in the composition (e.g., 85% or more of the plurality of conjugates in the composition) are each comprised of the same G-CSF moiety.

The composition can comprise a single conjugate species (e.g., a monoPEGylated conjugate wherein the single polymer is attached at the same location for substantially all conjugates in the composition) or a mixture of conjugate species (e.g., a mixture of monoPEGylated conjugates where attachment of the polymer occurs at different sites and/or a mixture monPEGylated, diPEGylated and triPEGylated conjugates). The compositions can also comprise other conjugates having four, five, six, seven, eight or more polymers attached to any given moiety having G-CSF activity. In addition, the invention includes instances wherein the composition comprises a plurality of conjugates, each conjugate comprising one water-soluble polymer covalently attached to one G-CSF moiety, as well as compositions comprising two, three, four, five, six, seven, eight, or more water-soluble polymers covalently attached to one G-CSF moiety.

With respect to the conjugates in the composition, the composition will satisfy one or more of the following characteristics: at least about 85% of the conjugates in the composition will have from one to four polymers attached to the G-CSF moiety; at least about 85% of the conjugates in the composition will have from one to three polymers attached to the G-CSF moiety; at least about 85% of the conjugates in the composition will have from one to two polymers attached to the G-CSF moiety; at least about 85% of the conjugates in the composition will have one polymer attached to the G-CSF moiety; at least about 95% of the conjugates in the composition will have from one to four polymers attached to the G-CSF moiety; at least about 95% of the conjugates in the composition will have from one to three polymers attached to the G-CSF moiety; at least about 95% of the conjugates in the composition will have from one to two polymers attached to the G-CSF moiety; at least about 95% of the conjugates in the composition will have one polymer attached to the G-CSF moiety; at least about 99% of the conjugates in the composition will have from one to four polymers attached to the G-CSF moiety; at least about 99% of the conjugates in the composition will have from one to three polymers attached to the G-CSF moiety; at least about 99% of the conjugates in the composition will have from one to two polymers attached to the G-CSF moiety; and at least about 99% of the conjugates in the composition will have one polymer attached to the G-CSF moiety.

In one or more embodiments, it is preferred that the conjugate-containing composition is free or substantially free of albumin. It is also preferred that the composition is free or substantially free of proteins that do not have G-CSF activity. Thus, it is preferred that the composition is 85%, more preferably 95%, and most preferably 99% free of albumin. Additionally, it is preferred that the composition is 85%, more preferably 95%, and most preferably 99% free of any protein that does not have G-CSF activity. To the extent that albumin is present in the composition, exemplary compositions of the invention are substantially free of of conjugates comprising a poly(ethylene glycol) polymer linking a residue of a G-CSF moiety to albumin.

Control of the desired number of polymers for any given moiety can be achieved by selecting the proper polymeric reagent, the ratio of polymeric reagent to the G-CSF moiety, temperature, pH conditions, and other aspects of the conjugation reaction. In addition, reduction or elimination of the undesired conjugates (e.g., those conjugates having four or more attached polymers) can be achieved through purification means.

For example, the polymer-G-CSF moiety conjugates can be purified to obtain/isolate different conjugated species. Specifically, the product mixture can be purified to obtain an average of anywhere from one, two, three, four, five or more PEGs per G-CSF moiety, typically one, two or three PEGs per G-CSF moiety. The strategy for purification of the final conjugate reaction mixture will depend upon a number of factors, including, for example, the molecular weight of the polymeric reagent employed, the particular G-CSF moiety, the desired dosing regimen, and the residual activity and in vivo properties of the individual conjugate(s).

If desired, conjugates having different molecular weights can be isolated using gel filtration chromatography and/or ion exchange chromatography. That is to say, gel filtration chromatography is used to fractionate differently numbered polymer-to-G-CSF moiety ratios (e.g., 1-mer, 2-mer, 3-mer, and so forth, wherein “1-mer” indicates 1 polymer to attached to a G-CSF moiety, “2-mer” indicates two polymers attached to a G-CSF moiety, and so on) on the basis of their differing molecular weights (where the difference corresponds essentially to the average molecular weight of the water-soluble polymer portion). For example, in an exemplary reaction where a 35,000 Dalton protein is randomly conjugated to a polymeric reagent having a molecular weight of about 20,000 Daltons, the resulting reaction mixture may contain unmodified protein (having a molecular weight of about 35,000 Daltons), monoPEGylated protein (having a molecular weight of about 55,000 Daltons), diPEGylated protein (having a molecular weight of about 75,000 Daltons), and so forth.

While this approach can be used to separate PEG and other polymer-G-CSF moiety conjugates having different molecular weights, this approach is generally ineffective for separating positional isoforms having different polymer attachment sites within the G-CSF moiety. For example, gel filtration chromatography can be used to separate from each other mixtures of 1-mers, 2-mers, 3-mers, and so forth, although each of the recovered conjugate compositions may contain PEG(s) attached to different reactive groups (e.g., lysine residues) within the G-CSF moiety.

Gel filtration columns suitable for carrying out this type of separation include Superdex™ and Sephadex™ columns available from Amersham Biosciences (Piscataway, N.J.). Selection of a particular column will depend upon the fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin (BSA) as a standard, (iii) iodine testing for PEG content (Sims et al. (1980) Anal. Biochem, 107:60-63), (iv) sodium dodecyl sulfate polyacrylamide gel electrophoresis (SDS PAGE), followed by staining with barium iodide, and (v) high performance liquid chromatography (HPLC).

Separation of positional isoforms is carried out by reverse phase chromatography using reverse phase-high performance liquid chromatography (RP-HPLC) using a suitable column (e.g., a C18 column or C3 column, available commercially from companies such as Amersham Biosciences or Vydac) or by ion exchange chromatography using an ion exchange column, e.g., a Sepharose™ ion exchange column available from Amersham Biosciences. Either approach can be used to separate polymer-active agent isomers having the same molecular weight (i.e., positional isoforms).

The compositions are preferably substantially free of proteins that do not have G-CSF activity. In addition, the compositions preferably are substantially free of all other noncovalently attached water-soluble polymers. In some circumstances, however, the composition can contain a mixture of polymer-G-CSF moiety conjugates and unconjugated G-CSF moiety.

In contrast to the compositions formed by the methods described in U.S. Patent Application Publication No. 2005/0143563, the presently described conjugate compositions are free or substantially free of aggregates. Consequently, the compositions of the invention are free or substantially free (e.g., less than about 20%, more preferably less than about 15%, still more preferably less than about 10%, yet still more preferably less than about 9%, yet still more preferably less than about 8%, yet still more preferably less than about 7%, yet still more preferably less than about 6%, yet still more preferably less than about 5%, yet still more preferably less than about 4%, yet still more preferably less than about 3%, yet still more preferably less than about 2%, yet still more preferably less than about 1%, with less than about 0.5% being most preferred) of aggregates.

An approach to address the formation of inactive aggregate formation is described in U.S. Patent Application Publication No. 2005/0143563. This reference describes treatment with a small amount of SDS, Tween20, Tween80 detergent is necessary to prevent the aggregates from being formed. Advantageously, the compositions and conjugates of the present invention can be prepared without performing the step of adding SDS, Tween20, and Tween80. In addition, compositions and conjugates of the present invention can be prepared without performing the step of adding a detergent. Furthermore, the compositions of the present invention are free or substantially free (e.g., less than about 20%, more preferably less than about 15%, still more preferably less than about 10%, yet still more preferably less than about 9%, yet still more preferably less than about 8%, yet still more preferably less than about 7%, yet still more preferably less than about 6%, yet still more preferably less than about 5%, yet still more preferably less than about 4%, yet still more preferably less than about 3%, yet still more preferably less than about 2%, yet still more preferably less than about 1%, yet still more preferably less than about 0.5%, with less than 0.001% being most preferred) of detergents such as SDS, Tween20, and Tween80. In addition, the compositions and conjugates of the present invention can be prepared without performing the step of removing (by, for example, ultra-filtration) detergents such as SDS, Tween20, and Tween80. Furthermore, the compositions and conjugates of the present invention can be prepared without performing the step of removing (by, for example, ultra-filtration) a detergent.

In contrast to the approach for forming conjugates in International Patent Application Publication No. WO 05/099769, the approach for preparing conjugates and compositions of the invention does not include the step of denaturing G-CSF to expose the thiol group of Cys-17. Preferably, the present methods for forming conjugates and compositions do not include the step of adding (and are not performed in the presence of) a denaturing agent, such as, for example, denaturing agents selected from the group consisting of urea, guanidine chloride or isothiocyanate, dimethylurea, high neurtal salt concentrations and solvents (such as for example, acetonitrile, alcohols, organic esters, dimethylsulfoxide). As shown in the Experimental, no such denaturing step is required to obtain conjugates of G-CSF at the Cys-17 residue.

Furthermore, the compositions of the present invention are free or substantially free (e.g., less than about 20%, more preferably less than about 15%, still more preferably less than about 10%, yet still more preferably less than about 9%, yet still more preferably less than about 8%, yet still more preferably less than about 7%, yet still more preferably less than about 6%, yet still more preferably less than about 5%, yet still more preferably less than about 4%, yet still more preferably less than about 3%, yet still more preferably less than about 2%, yet still more preferably less than about 1%, with less than about 0.5% being most preferred) of denaturing agent. In addition, the compositions and conjugates of the present invention can be prepared without performing the step of exposing the conjugate to renaturing conditions (such as, for example, ultra-filtration or chromatographic methods).

Optionally, the composition of the invention further comprises a pharmaceutically acceptable excipient. If desired, the pharmaceutically acceptable excipient can be added to a conjugate to form a composition.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for one or more embodiments of the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some situations, a surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate (i.e., the conjugate formed between the active agent and the polymeric reagent) in the composition will vary depending on a number of factors, but will optimally be a therapeutically effective dose when the composition is stored in a unit dose container (e.g., a vial). In addition, the pharmaceutical preparation can be housed in a syringe. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The compositions encompass all types of formulations and in particular those that are suited for injection, e.g., powders or lyophilates that can be reconstituted as well as liquids. Examples of suitable diluents for reconstituting solid compositions prior to injection include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

The compositions of one or more embodiments of the present invention are typically, although not necessarily, administered via injection and are therefore generally liquid solutions or suspensions immediately prior to administration. The pharmaceutical preparation can also take other forms such as syrups, creams, ointments, tablets, powders, and the like. Other modes of administration are also included, such as pulmonary, rectal, transdermal, transmucosal, oral, intrathecal, subcutaneous, intra-arterial, and so forth.

The invention also provides a method for administering a conjugate as provided herein to a patient suffering from a condition that is responsive to treatment with a conjugate as provided herein. The method comprises administering to a patient, generally via injection, a therapeutically effective amount of the conjugate (preferably provided as part of a pharmaceutical composition). As previously described, the conjugates can be administered parenterally by intravenous injection, or less preferably by intramuscular or by subcutaneous injection. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

The method of administering may be used to treat any condition that can be remedied or prevented by administration of the conjugate. Those of ordinary skill in the art appreciate which conditions the conjugates of the invention can effectively treat. For example, the conjugates can be used to treat patients suffering from myelosuppressive chemotherapy, a bone marrow transplant, severe chronic neutropenia, acquired immunodeficiency syndrome (AIDS), aplastic anemia, hairy cell leukemia, myelodysplasia, agranulocytosis (e.g., drug-induced agranulocytosis, congenital agranulocytosis, and alloimmune neonatalneutropenia). In addition, the conjugates can be used in patients in need of peripheral blood progenitor cell collection. Advantageously, a conjugate can be administered to the patient prior to, simultaneously with, or after administration of another active agent.

The actual dose to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 100 mg, preferably in doses from 0.01 mg/day to 75 mg/day, and more preferably in doses from 0.10 mg/day to 50 mg/day. A given dose can be periodically administered up until, for example, a desired (e.g., healthy) white blood cell count is achieved.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly, twice monthly, once monthly, and any combination thereof. Once the clinical endpoint has been achieved, dosing of the composition is halted.

One advantage of administering certain conjugates described herein is that individual water-soluble polymer portions can be cleaved. Such a result is advantageous when clearance from the body is potentially a problem because of the polymer size. Optimally, cleavage of each water-soluble polymer portion is facilitated through the use of physiologically cleavable and/or enzymatically degradable linkages such as amide, carbonate or ester-containing linkages. In this way, clearance of the conjugate (via cleavage of individual water-soluble polymer portions) can be modulated by selecting the polymer molecular size and the type functional group that would provide the desired clearance properties. One of ordinary skill in the art can determine the proper molecular size of the polymer as well as the cleavable functional group. For example, one of ordinary skill in the art, using routine experimentation, can determine a proper molecular size and cleavable functional group by first preparing a variety of polymer derivatives with different polymer weights and cleavable functional groups, and then obtaining the clearance profile (e.g., through periodic blood or urine sampling) by administering the polymer derivative to a patient and taking periodic blood and/or urine sampling. Once a series of clearance profiles have been obtained for each tested conjugate, a suitable conjugate can be identified.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### Experimental

The practice of the invention will employ, unless otherwise indicated, conventional techniques of organic synthesis, biochemistry, protein purification and the like, which are within the skill of the art. Such techniques are fully explained in the literature. See, for example, J. March, Advanced Organic Chemistry: Reactions Mechanisms and Structure, 4th Ed. (New York: Wiley-Interscience, 1992), supra.

In the following examples, efforts have been made to ensure accuracy with respect to numbers used (e.g., amounts, temperatures, etc.) but some experimental error and deviation should be taken into account. Unless indicated otherwise, temperature is in degrees C and pressure is at or near atmospheric pressure at sea level. Each of the following examples is considered to be instructive to one of ordinary skill in the art for carrying out one or more of the embodiments described herein.

Recombinant-methionyl human granulocyte-colony stimulating factor (G-CSF) is a non-glycosylated protein produced by E. coli and was used in Examples 1-5. The recombinant protein comprises of 175 amino acids with one free cysteine at position 17 (ignoring the leading methionine residue). The complete amino acid sequence is as follows: MTPLGPASSL PQSFLLKCLE QVRKIQGDGA ALQEKLCATY KLCHPEELVL LGHSLGIPWA PLSSCPSOAL QLAGCLSOLH SGLFLYQGLL QALEGISPEL GPTLDTLQLD VADFATTIWQ QMEELGMAPA LOPTQGAMPA FASAFORRAG GVLVASHLQS FLEVSYRVLR HLAQP, and corresponds to SEQ ID NO: 1, wherein n′″ is 1.

SDS-PAGE Analysis

When SDS-PAGE analysis was conducted, samples were analyzed by sodium dodecyl sulfate-polyacrylamide gel electrophoresis (SDS-PAGE) using Bio-Rad system (Mini-PROTEAN III Precast Gel Electrophoresis System), and Invitrogen system (XCell SureLock Mini-Cell). Samples were mixed with sample buffer. Then, the prepared samples were loaded onto a gel and run for approximately thirty minutes.

RP-HPLC Analysis

When RP-HPLC analysis was conducted for Examples 1A, 2B, 3A and 6, reversed phase high-performance liquid chromatography (RP-HPLC) was performed on an Agilent 1100 HPLC system (Agilent). Samples were analyzed using a PRP-3 column (3 μm particle size, 75×4.6 mm, Hamilton), and mobile phases consisting of 0.1% trifluoroacetic acid in water (buffer A) and 0.1% trifluoroacetic acid in acetonitrile (buffer B). The flow rate for the column was 0.5 ml/min. The protein and PEG-protein conjugates were eluted with a linear gradient over 40 minutes, and were visualized using UV detection at 280 nm.

When RP-HPLC analysis was conducted for Examples 1B, 1C and 1D, reversed phase high-performance liquid chromatography (RP-HPLC) was performed on an Agilent 1100 HPLC system (Agilent). Samples were analyzed using a Zorbax 300SB-C3 column (3.5 μm particle size, 150 mm×3.0 mm, Agilent), and mobile phases consisting of 0.1% trifluoroacetic acid in water (buffer A) and 0.1% trifluoroacetic acid in acetonitrile (buffer B). The flow rate for the column was 0.3 ml/min. The protein and PEG-protein conjugates were Eluted with a linear gradient over 35 minutes, and were detected using UV at 280 nm.

When present, dimers identified through RP-HPLC indicate protein dimer aggregates (and lack any polymeric component).

Cation Exchange Chromatography

When cation exchange chromatography was conducted, a HiTrap SP Sepharose HP cation exchange column (Amersham Biosciences) was used with the AKTAprime system (Amersham Biosciences) to purify the PEG-G-CSF conjugates. For each conjugate solution prepared, the conjugate solution was loaded on a column that was pre-equilibrated in 20 mM NaOAc buffer, pH 4.0 (buffer A) and then washed with ten column volumes of buffer A to remove any unreacted PEG reagent. Subsequently, a gradient of buffer A with 0-100% buffer B (20 mM NaOAc with 1.0 M NaCl buffer, pH 4.0) was raised. The eluent was monitored by liV detector at 280 nm. The fractions were pooled and the purity of the individual conjugate was determined by RP-HPLC or SDS-PAGE.

Percent Yields and Conjugate Solutions

Percent yields of PEGylation refer to the yield of monoPEGylated species. The terms “conjugate solution” and “reaction mixture” are the synonymous, and both represent the composition resulting from the described reaction or process.

### EXAMPLE 1A

### PEGylation of G-CSF with a Linear mPEG-Orthopyridyl-Disulfide Reagent (mPEG-OPSS), 10 kDa

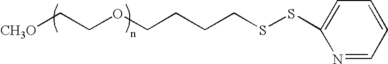

### Linear mPEG-Orthopyridyl-Disulfide Reagent (“mPEG-OPSS”), 10 kDa

mPEG-OPSS, 10 kDa, stored at −20° C. under argon, was warmed to ambient temperature. A fifty-fold excess (relative to the amount of G-CSF in a measured aliquot of the stock G-CSF solution) of the warmed mPEG-OPSS was dissolved in dimethylsulfoxide (“DMSO”) to form a 10% reagent solution. The 10% reagent solution was quickly added to the aliquot of stock G-CSF solution (0.4 mg/ml in sodium phosphate buffer, pH 7.0) and mixed well. To allow for coupling of the mPEG-OPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque IA) to facilitate conjugation at 37° C. After thirty minutes, another fifty-fold excess of mPEG-OPSS, 10 kDa, was added to the reaction solution, followed by mixing first for thirty minutes at 37° C., and then for two hours at room temperature to thereby form an mPEG10 kDa-G-CSF conjugate solution. The mPEG10 kDa-G-CSF conjugate solution was characterized by SDS-PAGE and RP-HPLC.

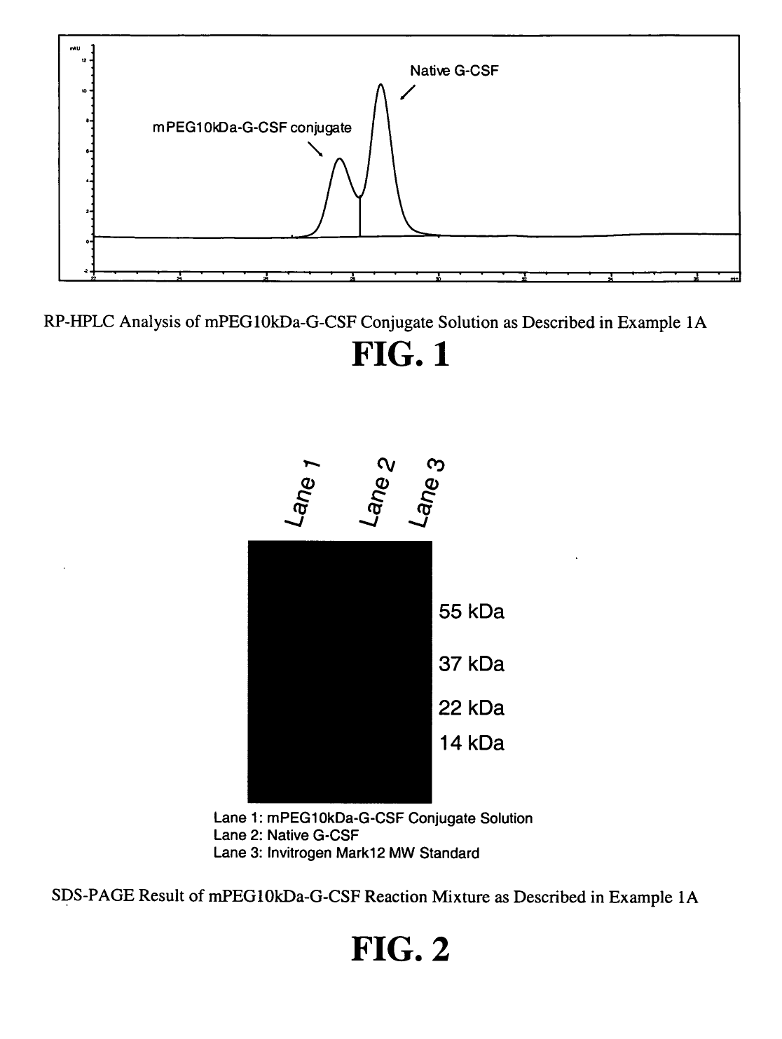

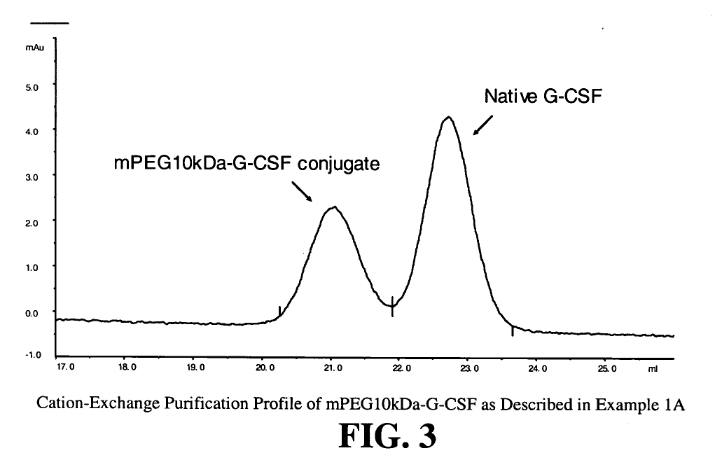

FIG. 1 shows the chromatogram following the RP-HPLC analysis of the mPEG10 kDa-G-CSF conjugate solution. The PEGylation reaction yielded 36% of mPEG10 kDa-G-CSF conjugate (a monoPEGylated conjugate at a cysteine residue of G-CSF). FIG. 2 shows SDS-PAGE analysis of the mPEG10 kDa-G-CSF conjugate solution. Cation-exchange chromatography was used to purify the conjugate. FIG. 3 shows the chromatogram following cation-exchange purification.

Using this same approach, other conjugates can be prepared using mPEG-OPSS having other weight average molecular weights.

### EXAMPLE 1B

### PEGylation of G-CSF with a Linear mPEG-Orthopyridyl-Disulfide Reagent (mPEG-OPSS), 10 kDa

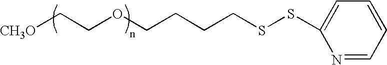

### Linear mPEG-Orthopyridyl-Disulfide Reagent (“mPEG-OPSS”), 10 kDa

mPEG-OPSS, 10 kDa, stored at −20° C. under argon, was warmed to ambient temperature. A fifty-fold excess (relative to the amount of G-CSF in a measured aliquot of the stock G-CSF solution) of the warmed mPEG-OPSS was dissolved in 50% DMSO to form a 10% reagent solution. The 10% reagent solution was quickly added to the aliquot of stock G-CSF solution (3.0 mg/ml in 10 mM sodium phosphate buffer, 1% (w/v) sucrose, pH 6.7) and mixed well. To allow for coupling of the mPEG-OPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa) to facilitate conjugation for one hour at 37° C., and then overnight at room temperature to thereby form an mPEG10 kDa-G-CSF conjugate solution. The mPEG10 kDa-G-CSF conjugate solution was characterized by SDS-PAGE and RP-HPLC.

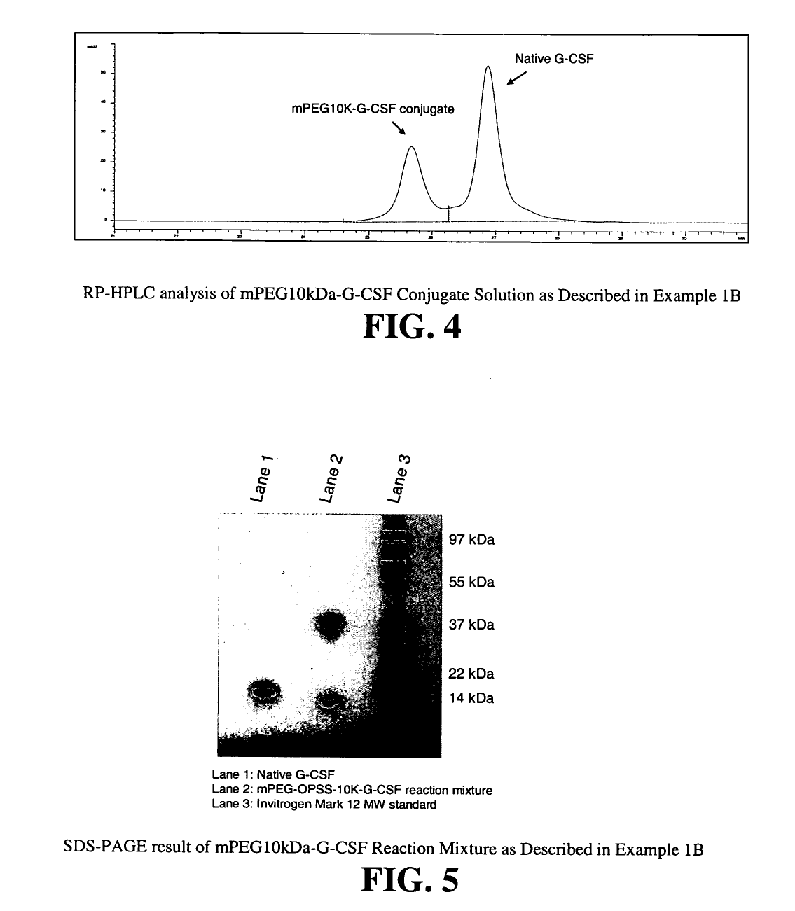

FIG. 4 shows the chromatogram following the RP-HPLC analysis of the conjugate solution. The PEGylation reaction yielded 34% mPEG10K-G-CSF conjugate.

FIG. 5 shows SDS-PAGE analysis of the conjugate solution.

Using this same approach, other conjugates can be prepared using mPEG-OPSS having other weight average molecular weights.

### EXAMPLE 1C

### PEGylation of G-CSF with a Linear mPEG-Orthopyridyl-Disulfide Reagent (mPEG-OPSS), 10 kDa

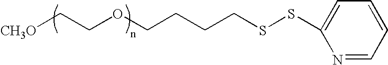

### Linear mPEG-Orthopyridyl-Disulfide Reagent (“mPEG-OPSS”), 10 kDa

mPEG-OPSS, 10 kDa, stored at −20° C. under argon, was warmed to ambient temperature. Warmed mPEG-OPSS (37 mg) was dissolved in acetonitrile to form a reagent solution. The reagent solution was quickly added to 1 ml of G-CSF solution (0.5 mg/ml in sodium phosphate buffer, pH 6.9) and mixed well. To allow for coupling of the mPEG-OPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa) to facilitate conjugation for 30 minutes at 37° C., and then for two hours at room temperature to thereby form an mPEG10 kDa-G-CSF conjugate solution. The mPEG10 kDa-G-CSF conjugate solution was characterized by RP-HPLC.

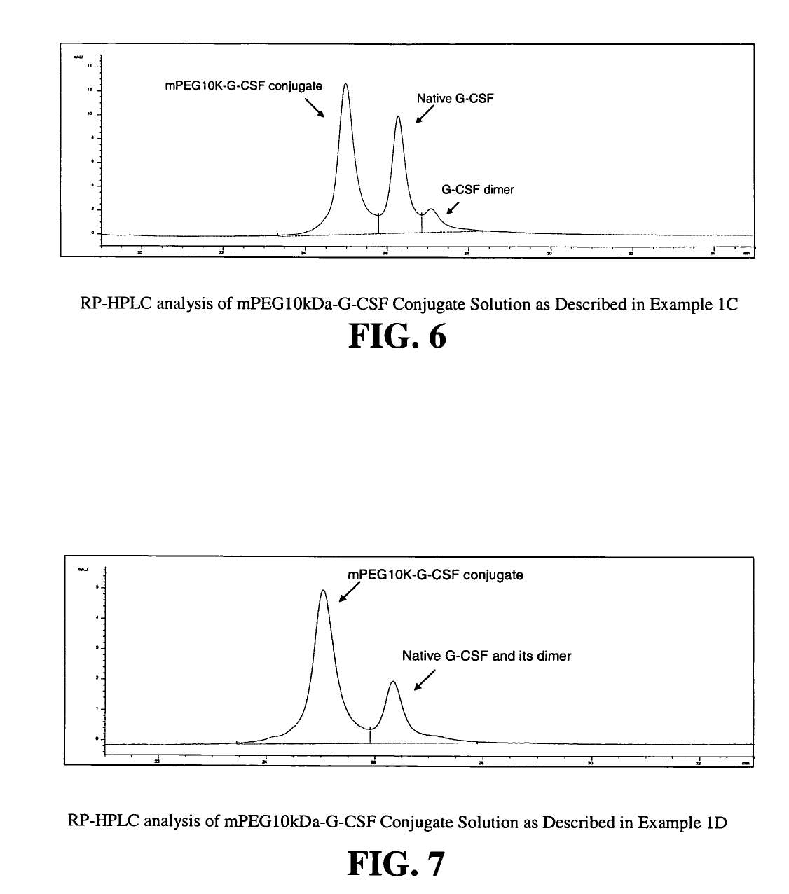

FIG. 6 shows the chromatogram following the RP-HPLC analysis of the mPEG10 kDa-G-CSF conjugate solution. The PEGylation reaction yielded 56% mPEG10K-G-CSF conjugate.

Using this same approach, other conjugates can be prepared using mPEG-OPSS having other weight average molecular weights.

### EXAMPLE 1D

### PEGylation of G-CSF with a Linear mPEG-Orthopyridyl-Disulfide Reagent (mPEG-OPSS), 10 kDa

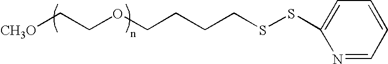

### Linear mPEG-Orthopyridyl-Disulfide Reagent, 10 kDa (“mPEG-OPSS”)

mPEG-OPSS, 10 kDa, stored at −20° C. under argon, was warmed to ambient temperature. Warmed mPEG-OPSS (17 mg) was dissolved in acetonitrile to form a reagent solution. The reagent solution was quickly added to 0.2 ml of G-CSF solution (0.3 mg/ml in 10 mM sodium phosphate buffer, 1% (w/v) sucrose, pH 7.0) and mixed well. To allow for coupling of the mPEG-OPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa) to facilitate conjugation for one hour at 37° C., and then for two hours at room temperature to thereby form an mPEG10 kDa-G-CSF. The mPEG10 kDa-G-CSF conjugate solution was characterized by RP-HPLC.

FIG. 7 shows the chromatogram following the RP-HPLC analysis of the mPEG10 kDa-G-CSF conjugate solution. The PEGylation reaction yielded 73% MPEG10K-G-CSF conjugate.

Using this same approach, other conjugates can be prepared using mPEG-OPSS having other weight average molecular weights.

### EXAMPLE 2A

### PEGylation of G-CSF with a Linear PEG-Diorthopyridyl-Disulfide Reagent, 2 kDa, and a Linear mPEG-Thiol Reagent, 20 kDa

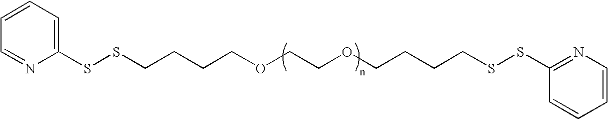

### Linear PEG-Diorthopyridyl-Disulfide Reagent, 2 kDa (“PEG-DiOPSS”)

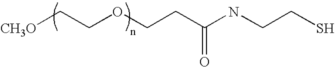

### Linear mPEG-Thiol Reagent, 20 kDa (“mPEG-SH”)

This Example (as well as Example 2B) relied on an approach involving initial attachment of a polymeric reagent having a relatively small weight average molecular weight to a G-CSF moiety followed by attachment of a relatively large weight average molecular weight polymeric reagent to the polymeric portion of the conjugate formed from attachment of the relatively small weight average molecular weight polymeric reagent to the G-CSF moiety. By taking this approach, it was possible to modify the partially buried free thiol-containing cysteine residue of G-CSF. The bifunctional PEG-DiOPSS, 2 kDa, was essentially inserted into the sterically hindered free thiol via a disulfide linkage, followed by the coupling of a thiol-terminated PEG to the exposed residue of the PEG-OPSS, 2 kDa, reagent through another disulfide linkage.

Schematically, the approach is shown below [wherein the polymeric reagent having a relatively low weight average molecular weight “PEG_{B}” is initially attached to a moiety to be conjugated (A), followed by attachment of a higher weight average molecular weight polymeric reagent (PEG_{A} in the schematic) to the polymeric portion of the conjugate formed from attachment of the low weight average molecular weight reagent to the conjugated moiety] Note that the structures provided below are merely illustrative and polymeric reagents of a variety of structures can be used.

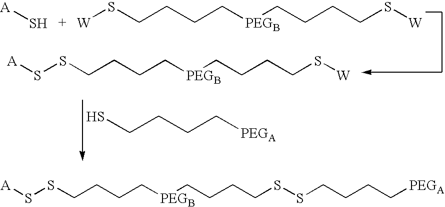

PEG-DiOPSS, 2 kDa, stored at −20° C. under argon, was warmed to ambient temperature. A fifty-fold excess (relative to the amount of G-CSF in a measured aliquot of the stock G-CSF solution) of the warmed PEG-DiOPSS was dissolved in DMSO to form a 10% reagent solution. The 10% reagent solution was quickly added to the aliquot of stock G-CSF solution (0.4 mg/ml in sodium phosphate buffer, pH 7.0) and mixed well. To facilitate the conjugation of the PEG-DIOPSS to the free (i.e., nonintraprotein-disulfide bond participating) cysteine residue at position 17 of G-CSF via a disulfide linkage, the reaction solution was placed on a RotoMix (Type 48200, Then-nolyne, Dubuque Iowa), and was allowed to mix for one hour at 37° C., and then for two hours at room temperature to thereby result in a PEG2 kDa-G-CSF reaction mixture. After the reaction was complete, the reaction solution was dialyzed against a sodium phosphate buffer, pH 7.0, to remove the excess free PEG-DiOPSS. A fifty-fold excess of mPEG-SH, 20 kDa (relative to the amount of G-CSF in a measured aliquot of the stock G-CSF solution) was then added to the dialyzed conjugate solution, followed by mixing for one hour at room temperature and then overnight at 4° C. to thereby form an mPEG20 kDa-PEG2 kDa-G-CSF conjugate solution. The n-PEG20 kDa-PEG2 kDa-G-CSF conjugate solution was characterized by SDS-PAGE and RP-BPLC.

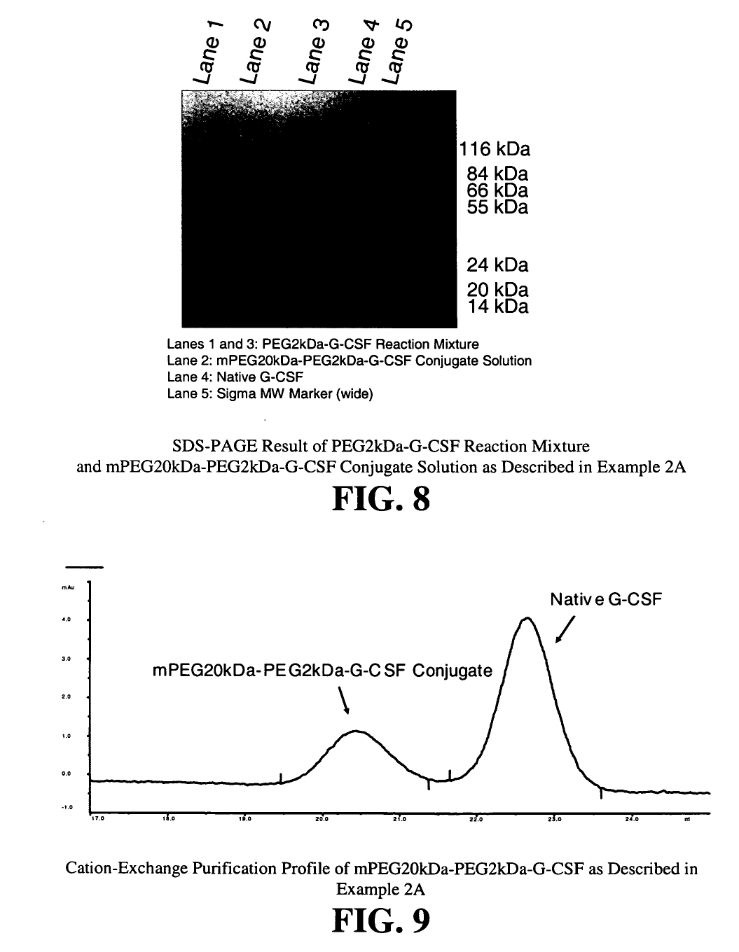

FIG. 8 shows SDS-PAGE analysis of the mPEG20 kDa-PEG2 kDa-G-CSF conjugate solution. The first step of PEGylation with PEG-diOPSS yielded 58% PEG2 kDa-G-CSF conjugate, while the second step of reaction with mPEG-SH yielded 42% mPEG20 kDa-PEG2 kDa-G-CSF conjugate.

Cation-exchange chromatography was used to purify the final conjugate. FIG. 9 shows the chromatogram following cation-exchange purification.

Using this same approach, other conjugates can be prepared using PEG-OPSS and mPEG-SH having other weight average molecular weights.

### EXAMPLE 2B

### PEGylation of G-CSF with a Linear PEG-Diorthopyridyl-Disulfide Reagent, 2 kDa, and a linear mPEG-Thiol Reagent, 20 kDa

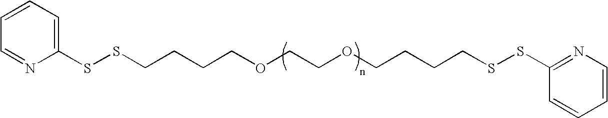

### Linear PEG-Diorthopyridyl-Disulfide Reagent, 2 kDa (“PEG-DiOPSS”)

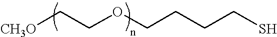

### Linear mPEG-Thiol Reagent, 20 kDa (“mPEG-SH”)

PEG-DiOPSS, 2 kDa, stored at −20° C. under argon, was warmed to ambient temperature. A one hundred-fold excess (relative to the amount of G-CSF in a measured aliquot of the stock G-CSF solution) of the warmed PEG-DiOPSS was dissolved in DMSO to form a 10% reagent solution. The 10% reagent solution was quickly added to the aliquot of stock G-CSF solution (0.5 mg/ml in sodium phosphate buffer, pH 7.0) and mixed well. To facilitate the conjugation of the PEG-DiOPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa), and was allowed to mix for one hour at 37° C., and then for three and a half hours at room temperature. After the reaction was complete, the reaction solution was dialyzed against the sodium phosphate buffer, pH 7.0 to remove the excess free PEG-DiOPSS. Thereafter, a one hundred-fold excess of mPEG-SH, 20 kDa (relative to the amount of G-CSF in a measured aliquot of the stock G-CSF solution) was then added to the dialyzed conjugate solution, followed by mixing for overnight at room temperature to thereby form an mPEG20 kDa-PEG2 kDa-G-CSF conjugate solution. The mPEG20 kDa-PEG2 kDa-G-CSF conjugate solution was characterized by SDS-PAGE and RP-HPLC.

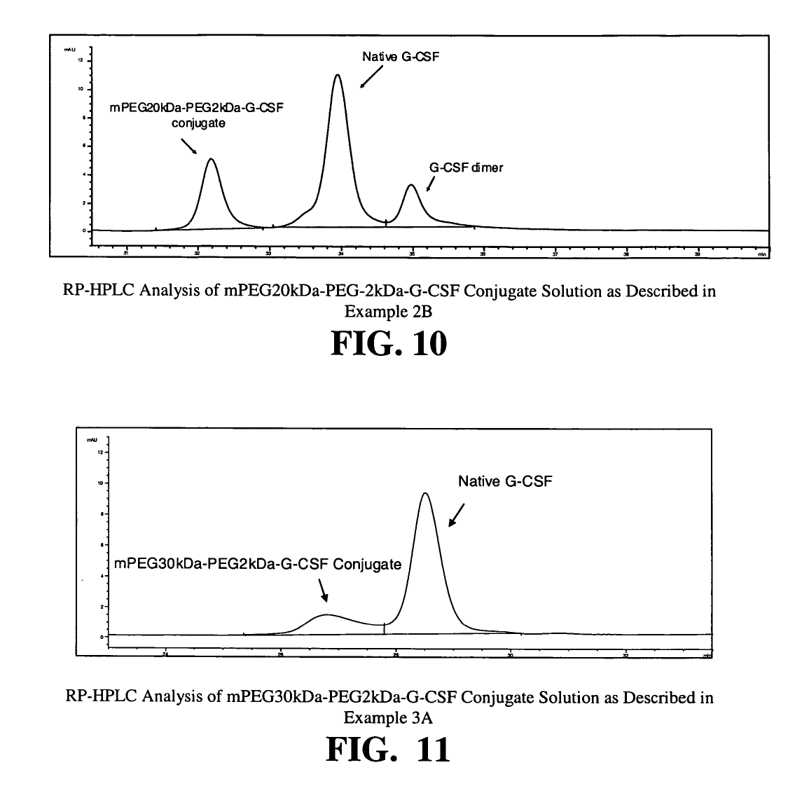

FIG. 10 shows the chromatogram following the RP-HPLC analysis of the conjugate solution. The PEGylation reaction yielded 25% mPEG20 kDa-PEG2 kDa-G-CSF conjugate.

A cation-exchange chromatography method using SP Sepharose High Performance exchange media (Amersham Biosciences, Uppsala Sweden) and NaOAc buffer was used to purify the mPEG20 kDa-PEG2 kDa-G-CSF conjugate.

Using this same approach, other conjugates can be prepared using PEG-DiOPSS and MPEG-SH having other weight average molecular weights.

### EXAMPLE 3A

### PEGylation of G-CSF with Linear PEG-Diorthopyridyl-Disulfide Reagent, 2 kDa, and a Linear mPEG-Thiol Reagent, 30 kDa

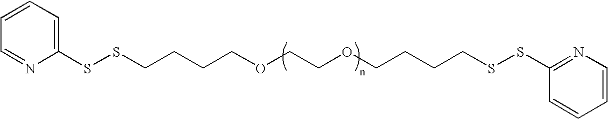

### Linear PEG-DiOrthopyridyl-Disulfide Reagent, 2 kDa (“PEG-DiOPSS”)

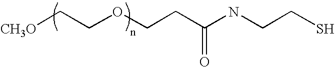

### Linear mPEG-Thiol Reagent, 30 kDa (“mPEG-SH”)

This Example (as well as Example 3B) relied on an approach involving initial attachment of a polymeric reagent having a relatively small weight average molecular weight to a G-CSF moiety followed by attachment of a relatively large weight average molecular weight polymeric reagent to the polymeric portion of the conjugate formed from attachment of the relatively small weight average molecular weight polymeric reagent to the G-CSF moiety. By taking this approach, it was possible to modify the partially buried free thiol-containing cysteine residue of G-CSF. The bifunctional PEG-DiOPSS, 2 kDa, was essentially inserted into the sterically hindered free thiol via a disulfide linkage, followed by the coupling of a thiol-terminated PEG to the residue of the PEG-OPSS, 2 kDa, reagent through another disulfide linkage.

PEG-DiOPSS, 2 kDa, stored at −20° C. under argon, was warmed to ambient temperature. A fifty-fold excess (relative to the amount of G-CSF in a measured aliquot of the stock G-CSF solution) of the warmed PEG-DiOPSS was dissolved in DMSO to form a 10% reagent solution. The 10% reagent solution was quickly added to the aliquot of stock G-CSF solution (0.4 mg/ml in sodium phosphate buffer, pH 7.0) and mixed well. To facilitate the conjugation of PEG-DiOPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa), and was allowed to mix for one hour at 37° C., and then for two hours at room temperature to thereby result in a PEG2 kDa-G-CSF reaction mixture. After the reaction was complete, the reaction solution was dialyzed against the sodium phosphate buffer, pH 7.0, to remove the excess free PEG-DiOPSS. A fifty-fold excess of mPEG-SH, 30 kDa (relative to the amount of G-CSF in a measured aliquot of the stock G-CSF solution) was then added to the dialyzed conjugate solution, followed by mixing for one hour at room temperature and then overnight at 4° C. to thereby form an mPEG3OkDa-PEG2 kDa-G-CSF conjugate solution. The mPEG30 kDa-PEG2 kDa-G-CSF conjugate solution was characterized by SDS-PAGE and RP-HPLC.

FIG. 11 shows the chromatogram following the RP-HPLC analysis of the mPEG30 kDa-PEG2 kDa-G-CSF conjugate solution. The PEGylation reaction yielded 20% of mPEG30 kDa-PEG2 kDa-G-CSF conjugate.

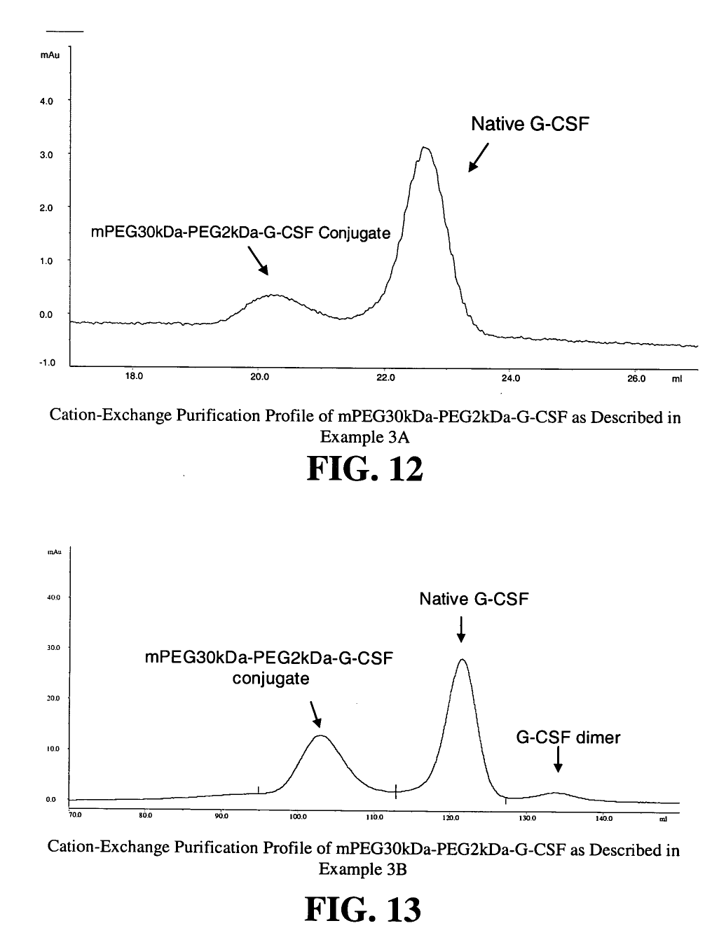

Cation-exchange chromatography was used to purify the mPEG30 kDa-PEG2 kDa-G-CSF conjugate. FIG. 12 shows the chromatogram following cation-exchange purification.

Using this same approach, other conjugates can be prepared using PEG-OPSS and mPEG-SH having other weight average molecular weights.

### EXAMPLE 3B

### PEGylation of G-CSF with a Linear PEG-Diorthopyridyl-Disulfide Reagent, 2 kDa, and a linear mPEG-Thiol Reagent, 30 kDa

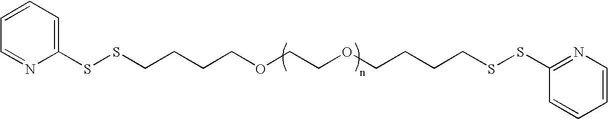

### Linear PEG-Orthopyridyl-Disulfide Derivative, 2 kDa (“PEG-DiOPSS”)

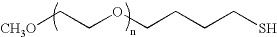

### Linear mPEG-Thiol Derivative, 30 kDa (“mPEG-SH”)

PEG-DiOPSS, 2 kDa, stored at −20° C. under argon, was warmed to ambient temperature. A one hundred-fold excess (relative to the amount of G-CSF in a measured aliquot of the stock G-CSF solution) of the warmed PEG-DIOPSS, 2 kDa was dissolved in DMSO to form a 10% reagent solution. The 10% reagent solution was quickly added to the aliquot of stock G-CSF solution (0.5 mg/ml in sodium phosphate buffer, pH 7.0) and mixed well. To facilitate the conjugation of PEG-DiOPSS to the free (i.e., nonintraprotein-disulfide bond participating) cysteine residue at position 17 of G-CSF via a disulfide linkage, the reaction solution was placed on a RotoMix (Type 48200, Thermolyne, Dubuque Iowa), and was allowed to mix for one hour at 37° C., and then for three and a half hours at room temperature. After the reaction was complete, the reaction solution was dialyzed against the sodium phosphate buffer, pH 7.0 to remove the excess free PEG-DiOPSS. One hundred and fifty-fold excess of mPEG-SH, 30 kDa (relative to the amount of G-CSF in a measured aliquot of the stock G-CSF solution) was then added to the dialyzed conjugate solution, followed by mixing for overnight at room temperature to thereby form an mPEG30 kDa-PEG-2 kDa-G-CSF conjugate solution. The conjugate solution was characterized by SDS-PAGE and RP-HPLC.

The PEGylation reaction yielded 21% mPEG30 kDa-PEG2 kDa-G-CSF conjugate.

A cation-exchange chromatography method using SP Sepharose High Performance exchange media (Amersham Biosciences, Uppsala Sweden) and NaOAc buffer was used to purify the mPEG30K-PEG2K-G-CSF conjugate (See FIG. 13).

Using this same approach, other conjugates can be prepared using PEG-DiOPSS and mPEG-SH having other weight average molecular weights.

### EXAMPLE 4

### Degradable PEGylation of G-CSF with 9-Hydroxymethyl-2,7-Di[mPEG(20,000)-Amidoglutaric Amide]Fluorene-N-Hydroxysuccininide Reagent (a “Branched” Reagent), 40 kDa

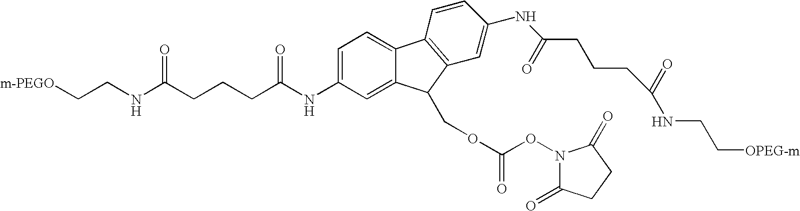

### 9-Hydroxymethyl-2,7-Din[mPEG(20,000)-Amidoglutaric Amide]Fluorene-N-Hydroxysuccinimide Reagent, 40 kDa or “Branched mPEG-FMOC-N-Hydroxysuccinimide Reagent”, 40 kDa or “G2-PEG2-FMOC-NHS”, 40 kDa

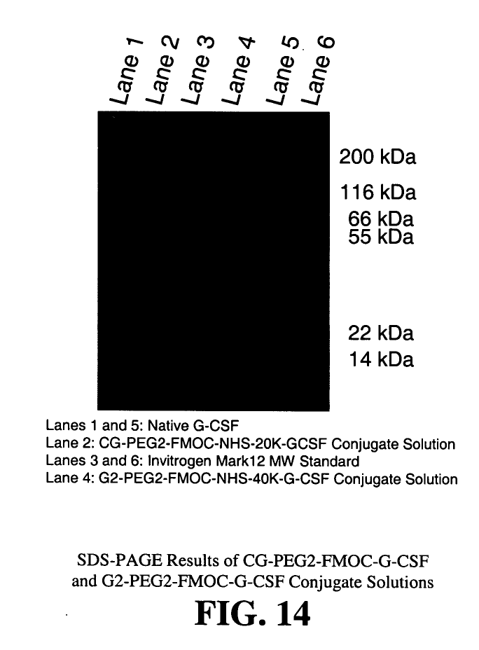

G2-PEG2-FMOC-NHS, 40 kDa, stored at −20° C. under argon, was warmed to ambient temperature. A five-fold excess (relative to the amount of G-CSF in a measured aliquot of the stock G-CSF solution) of the warmed G2-PEG2-FMOC-NHS was dissolved in 2 mM HCl to form a 10% reagent solution. The 10% reagent solution was quickly added to the aliquot of stock G-CSF solution (0.4 mg/ml in sodium phosphate buffer, pH 7.0) and mixed well. After the addition of the PEG reagent, the pH of the reaction mixture was determined and adjusted to 7.0 using conventional techniques. To allow for coupling of the G2-PEG2-FMOC-NHS to G-CSF via an amide linkage, the reaction solution was placed on a Slow Speed Lab Rotator for three hours to facilitate conjugation at room temperature to thereby form a G2-PEG2-FMOC-G-CSF conjugation solution. The reaction was quenched by the addition of 1M acetic acid to lower the pH to 4.0. The G2-PEG2-FMOC-G-CSF conjugate solution was characterized by SDS-PAGE. See lane 4 of the SDS-PAGE results provided in FIG. 14.

The PEGylation reaction yielded 52% 1-mer (mono-conjugate or one PEG attached to G-CSF) and 15% 2-mer (di-conjugate or two PEGs attached to G-CSF) species. A cation-exchange chromatography method using SP Sepharose High Performance exchange media and NaOAc (sodium acetate) buffer was used to purify the conjugates.

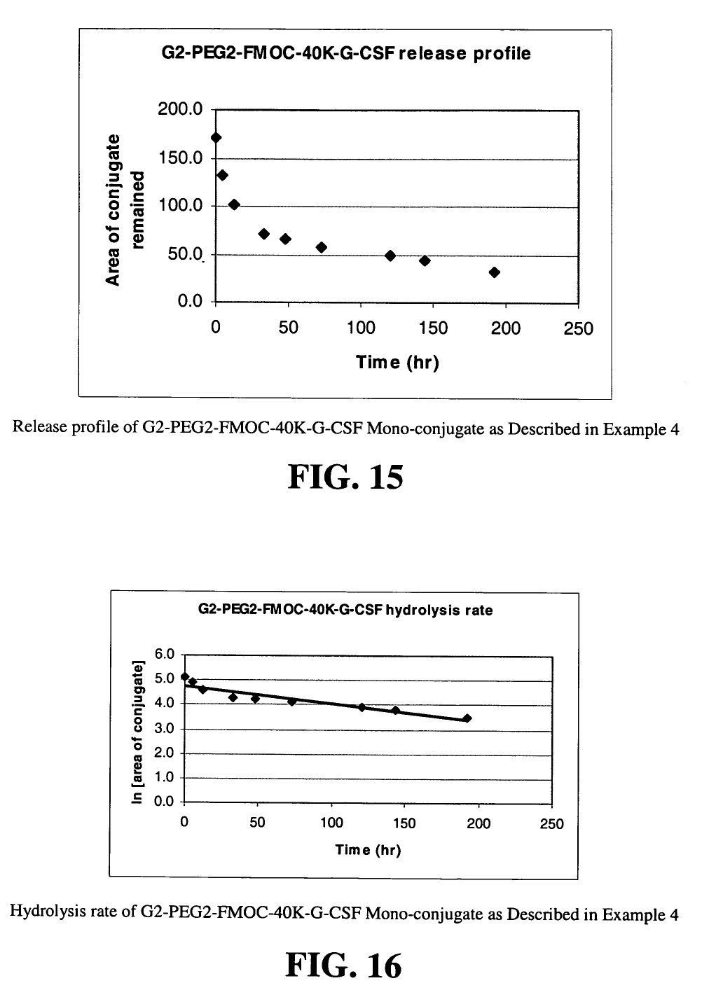

Due to the degradable linkage in the PEG structure, the G-CSF is expected to be released in a slow rate from the conjugates under physiological conditions. Evidence for this is shown in the release profile of the G2-PEG2-FMOC-40K-G-CSF mono-conjugate (see FIG. 15) upon incubation at pH 7.4, 37° C., expressed as the HPLC peak area of the conjugate remained as a function of time. The half time of the G2-PEG2-FMOC-40K-G-CSF mono-conjugate was calculated as 98 hours from the linear plot of the hydrolysis rate (see FIG. 16).

Using this same approach, other conjugates can be prepared using G2-PEG2-FMOC-NHS reagents having other weight average molecular weights.

### EXAMPLE 5

### Degradable PEGylation of G-CSF with 9-Hydroxymethyl-[4-Carboxamido mPEG(10,000)-7-Amidoglutaric Amide mPEG(10,000)]Fluorene-N-Hydroxysuccininide Reagent, 20 kDa

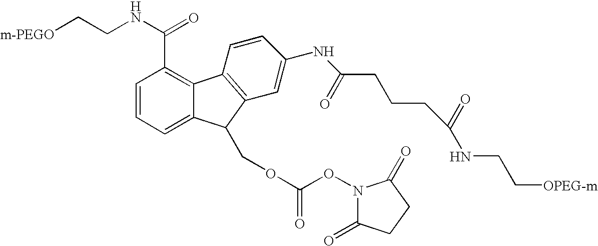

### 9-Hydroxymethyl-[4-Carboxamido mPEG(10,000)-7-Amidoglutaric Amide mPEG(10,000)]Fluorene-N-Hydroxysuccinimide Reagent, 20 kDa or “Branched mPEG-FMOC-N-Hydroxysuccinimide Reagent”, 20 kDa, or “CG-PEG2-FMOC-NHS”, 20 kDa

CG-PEG2-FMOC-NHS, 20 kDa, stored at −20° C. under argon, was warmed to ambient temperature. A seven-fold excess (relative to the amount of G-CSF in a measured aliquot of the stock G-CSF solution) of the warmed CG-PEG2-FMOC-NHS was dissolved in 2 mM HCl to form a 10% reagent solution. The 10% reagent solution was quickly added to the aliquot of stock G-CSF solution (0.4 mg/ml in sodium phosphate buffer, pH 7.0) and mixed well. After the addition of the PEG reagent, the pH of the reaction mixture was determined and adjusted to 7.0 using conventional techniques. To allow for coupling of the CG-PEG2-FMOC-NHS to G-CSF via an amide linkage, the reaction solution was placed on a Slow Speed Lab Rotator for three hours to facilitate conjugation at room temperature to thereby form a CG-PEG2-FMOC-G-CSF conjugate solution. The reaction was quenched by the addition of 1M acetic acid to lower the pH to 4.0. The CG-PEG2-FMOC-G-CSF conjugate solution was characterized by SDS-PAGE. See lane 2 of SDS-PAGE results provided in FIG. 14.

The PEGylation reaction yielded 45% 1-mer (mono-conjugate or one PEG attached to G-CSF) and 26% 2-mer (di-conjugate or two PEGs attached to G-CSF) species. A cation-exchange chromatography method using SP Sepharose High Performance exchange media and NaOAc buffer was used to purify the conjugates.

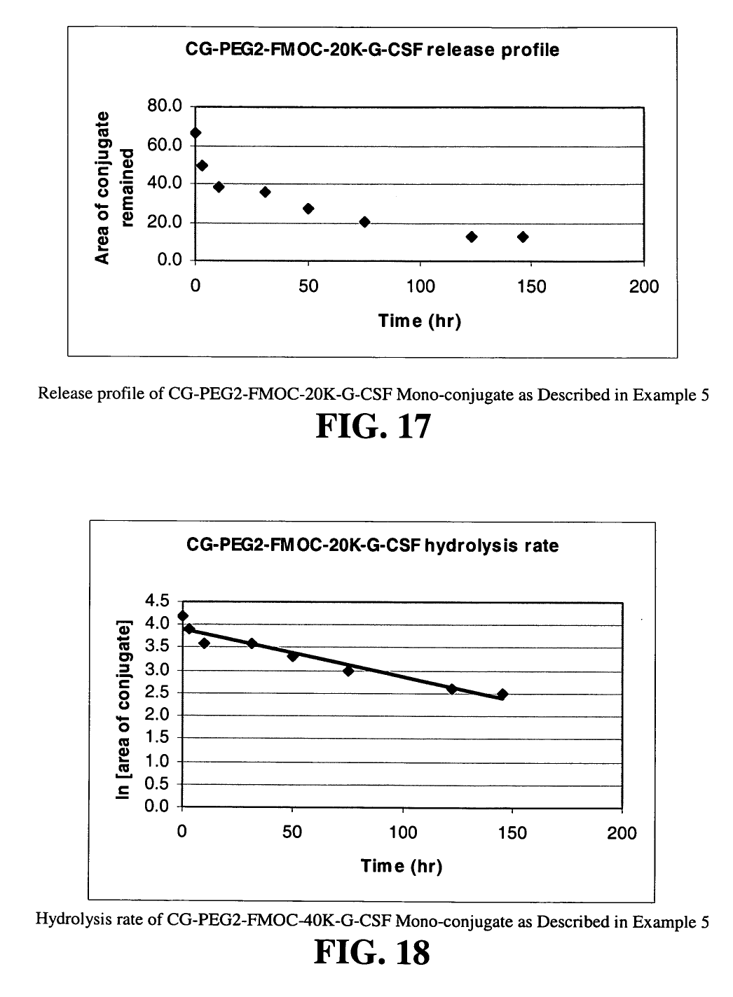

Due to the degradable linkage in the PEG structure, the G-CSF is expected to be slowly released from the PEG-G-CSF conjugates under physiological conditions. Evidence for this is shown in the release profile of CG-PEG2-FMOC-20K-G-CSF mono-conjugate (see FIG. 17) upon incubation at pH 7.4, 37° C., expressed as the HPLC peak area of the conjugate remained as a function of time. The half time of the CG-PEG2-FMOC-20K-G-CSF mono-conjugate was calculated as 60 hours from the linear plot of the hydrolysis rate (see FIG. 18).

Using this same approach, other conjugates can be prepared using CG-PEG2-FMOC-NHS reagents having other weight average molecular weights.

### EXAMPLE 6

### PEGylation of G-CSF with PEG₂₀₀₀-di-((CH₂)₄-orthopyridyl disulfide) and branched PEG2_{40,000}-thiol

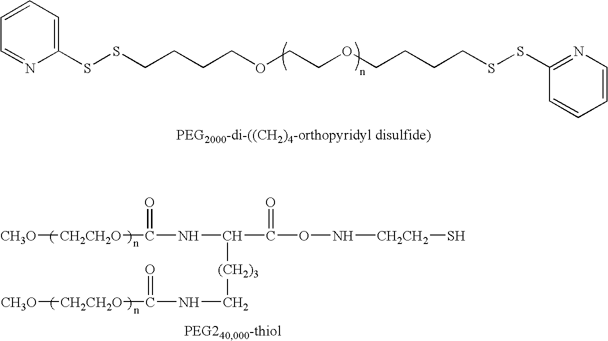

PEG_{2,000}-di-(4C-OPSS) (as prepared in Example 2 of U.S. Patent Application Publication No. 2006/0135586) stored at −20° C. under argon, was warmed to ambient temperature. A fifty-fold excess (relative to the amount of G-CSF in a measured aliquot of stock G-CSF solution) of the warmed PEG_{2,000}-di-(4C-OPSS) was dissolved in DMSO to form a 10% reagent solution. The 10% reagent solution was quickly added to the aliquot of stock G-CSF solution (0.4 mg/ml in sodium phosphate buffer, pH 7.0) and mixed well. The solution was placed on a RotoMix (Type 48200, Thermolyne, Dubuque Iowa) and allowed to mix for two hours at 37° C., then for two hours at room temperature. After the reaction was complete, the reaction solution was dialyzed against sodium phosphate buffer, pH 7.0, to remove excess PEG_{2,000}-di-(4C-OPSS).

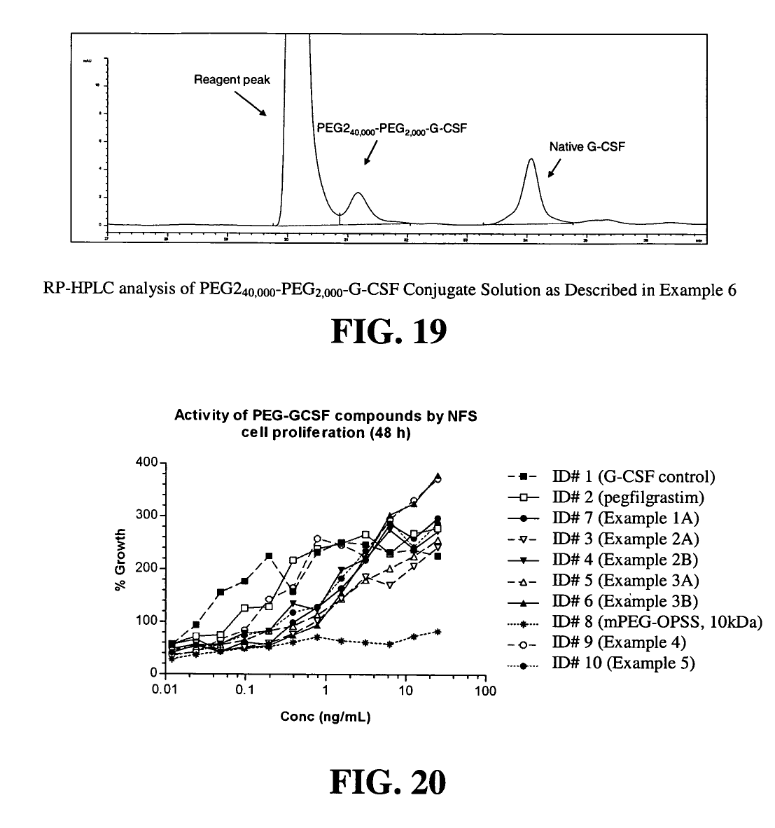

A seventy five-fold excess (relative to G-CSF) of PEG2_{40,000}-thiol (Nektar Therapeutics, Huntsville Ala.) was then added to the dialyzed conjugate solution, followed by mixing for three hours at room temperature and then overnight at 4° C., to form a PEG2_{40,000}-PEG_{2,000}G-CSF conjugate. The conjugate was characterized by SDS-PAGE and RP-HPLC. As shown in FIG. 19, the final yield of conjugate obtained was 35%.

### EXAMPLE 7 (COMPARATIVE)

### PEGylation Reaction of G-CSF with PEG₂₀₀₀-di-((CH₂).orthopyridyl disulfide) and PEG2_{40,000}-thiol

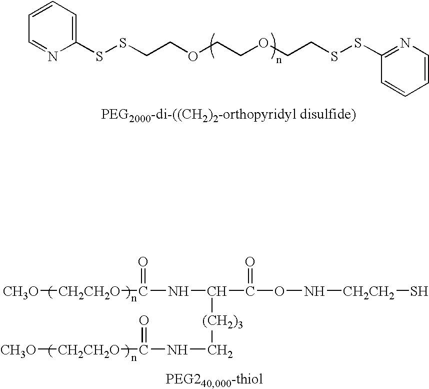

The reaction procedure of Example 6 was essentially duplicated, using a low molecular weight PEG thiol reagent having a two-carbon rather than a four-carbon linker.

Accordingly, PEG_{2,000}-di-(2C-OPSS) from Nektar Therapeutics, Huntsville Ala., stored at −20° C. under argon, was warmed to ambient temperature. A fifty-fold excess (relative to the amount of G-CSF in a measured aliquot of stock G-CSF solution) of the reagent was dissolved in DMSO to form a 10% solution. This solution was quickly added to the aliquot of stock G-CSF solution (0.4 mg/ml in sodium phosphate buffer, pH 7.0) and mixed well. The reaction solution was placed on a RotoMix (Type 48200, Thermolyne, Dubuque Iowa) and was allowed to mix for two hours at 37° C., then for two hours at room temperature. After the reaction was complete, the reaction solution was dialyzed against sodium phosphate buffer, pH 7.0, to remove excess PEG_{2,000}-di-(2C-OPSS).

A seventy five-fold excess (relative to G-CSF) of branched PEG2_{40,000}-thiol (Nektar Therapeutics, Huntsville Ala.) was added to the dialyzed conjugate solution, followed by mixing for three hours at room temperature and overnight at 4° C. However, SDS-PAGE and RP-HPLC analysis showed no detectable amount of the desired PEG2_{40,000}-PEG_{2,000}-G-CSF conjugate.

Evidence suggests that the ethylene (C2)-linked PEG-OPSS reagent undergoes reductive cleavage to effectively destroy the reagent before and after it reacts with the target protein. The butylene (C4)-linked reagent is more stable to such cleavage and thereby survives to give a much higher yield of conjugate.

### EXAMPLE 8

### PEGylation in Series of rhG-CSF

G-CSF is dissolved in a sodium acetate buffer, pH 6.8, to form a stock solution. About a forty molar excess (relative to the G-CSF) of OPSS-PEG_{2,000 Dalton}-hydrazide in water is added to the stock solution to form a reaction solution.

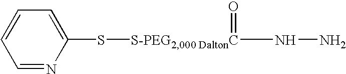

### OPSS-PEG_{2,000} Dalton-Hydrazide Reagent

To allow for reaction of a cysteine with the sulfhydryl reactive orthopyridyl disulfide group (“OPSS”), the reaction solution is mixed for three hours at room temperature. The reaction solution is then passed through a size-exclusion chromatography column and the peak associated with the monoPEGylated (“1-mer”) conjugate [having a structure of (G-CSF)-S-S-PEG_{2,000 Dalton}C(O)—NH—NH₂] are collected to form a monoPEGylated composition.

The monoPEGylated composition is then treated with a twenty molar excess of mPEG_{30,000 Dalton} propionaldehyde derivative to form a second reaction solution.

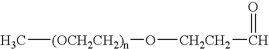

### mPEG Propionaldehyde Reagent

To allow for reaction between the hydrazide and aldehyde functional groups, the second reaction solution is mixed for three hours at room temperature at pH adjusted to 3.8. Analysis of the reaction mixture reveals successful conjugation of G-CSF having the following structure: (rG-CSF)-S-S-PEG_{2,000 Dalton} C(O)—NH—N═CHCH₂CH₂O(CH₂CH₂O)CH₃.

Using this same approach, other conjugates can be prepared using PEG-OPSS and MPEG- propionaldehyde reagents having other weight average molecular weights.

### EXAMPLE 9

### Activity of Conjugates

The objective of Example 9 was to evaluate the efficacy of conjugates of human recombinant granulocyte-colony stimulating factor (G-CSF) identified in Table 4.

TABLE 4
Summary of PEG-G-CSF Conjugates Prepared for In-vitro
and In-vivo Evaluation

| | | Protein | |
|---|---|---|---|
| | | Concentration | Volume |
| ID# | Sample name | (μg/ml) | (ml) |
| 01 | G-CSF control | 67.8 | 1.8 |
| 02 | pegfilgrastim* | 132.1 | 1.5 |
| 03 | Example 2A | 31.6 | 1.8 |
| 04 | Example 2B | 27.3 | 1.7 |
| 05 | Example 3A | 47.6 | 1.8 |
| 06 | Example 3B | 24.2 | 1.7 |
| 07 | Example 1A | 53.0 | 1.2 |
| 08 | mPEG-OPSS, | 27.6 | 1.5 |
| | 10 kDa, control | | |
| | (as shown in | | |
| | Example 1A) | | |
| 09 | Example 4 | 51.0 | 1.8 |
| 10 | Example 5 | 31.2 | 1.8 |

*Pegfilgrastim is a covalent conjugate of recombinant methionyl human G-CSF (filgrastim) and monomethoxypolyethylene glycol and is commercially available from Amgen Inc., Thousand Oaks CA. When administered subcutaneously to humans at 3.45-11.5 mcg/kg, it has been reported that filgrastim has a half life of 3.5 hours, a clearance of 0.5-0.7 mL/min/kg and a volume of distribution of 150 mL/kg. Pegfilgrastim, however, has been reported as having a half life of 15-80 hours.

M-NFS-60 mouse myeloma cells (ATCC #CRL-1838) were obtained and maintained in RPMI-1640 (ATCC #30-2001) supplemented with 10% FBS (HyClone), 50 μM 2-mercaptoethanol (Gibco) and 62 ng/ml human recombinant macrophage colony-stimulating factor (rhM-CSF, Sigma #M6518). Cells were subcultured every 2-3 days, or 3 times per week. Seed density was 2.5E4 cells/mL. The cells are not anchorage dependent. Just before testing the PEG-GCSF compounds, the cells were washed three times with PBS buffer to remove the rhM-CSF. The procedure in Table 5 was as followed.

TABLE 5
Procedure

| Day | Activity |
|---|---|
| Day 0 | Prepared cell suspension at 2.5E4 to 5E4 cells/mL in media without rhM-CSF. |
| | The cell suspension was added in 96-well plates, 100 μL per well. The test |
| | compounds were diluted in complete growth media (without rhM-CSF). |
| | The cells were treated with test compounds by adding 100 μL per well [2X dilution |
| | and in duplicate]. The final compound concentrations were 25, 12.5, 6.25, 3.1, 1.56, |
| | 0.78, 0.39, 0.195, 0.097, 0.049, 0.024 and 0.012 ng/mL. |
| | The seed density reduced to 1.25E4 or 2.5E4 cells/mL following drug addition. The |
| | negative media control was the compound solvent without rhM-CSF. |
| Day 1 | At 24 hours, the NFS-60 cell proliferation was determined on one set of plates by the |
| | MTT cell viability assay. |
| Day 2 | At 48 hours, the NFS-60 cell proliferation was determined on the 2^{nd} set of plates by |
| | the MTT cell viability assay. |
| Day 3 | At 72 hours, the NFS-60 cell proliferation was determined on the 3^{rd} set of plates by |
| | the MTT cell viability assay. |

The activity of each conjugate was determined pharmacodynamically in normal male Sprague-Dawley rats (Harlan, Indianapolis Ind.). The animals were ordered within the weight range 150-200 g and their approximate age was 6 weeks. They were acclimatized to the animal house for at least 3 days after delivery, before commencing the study investigation.

There were 10 treatment groups, with 4 rats per group. Each treatment group was given a letter (A-J) and the rats were arbitrarily allocated to each treatment group, as shown in Table 6.

TABLE 6
Treatment Groups

| Group | ID# | Dose | Concentration | Dose volume |
|---|---|---|---|---|
| A | 08 | 100 μg/kg | 71.2 μg/mL | 1.40 mL/kg |
| B | 07 | 40 μg/kg | 71.2 μg/mL | 0.56 mL/kg |
| C | 07 | 100 μg/kg | 71.2 μg/mL | 1.40 mL/kg |
| D | 04 | 40 μg/kg | 71.7 μg/mL | 0.56 mL/kg |
| E | 04 | 100 μg/kg | 71.7 μg/mL | 1.39 mL/kg |
| F | 06 | 40 μg/kg | 60.0 μg/mL | 0.67 mL/kg |
| G | 06 | 100 μg/kg | 60.0 μg/mL | 1.67 mL/kg |
| H | 09 | 40 μg/kg | 101.0 μg/mL | 0.40 mL/kg |
| I | 09 | 100 μg/kg | 101.0 μg/mL | 0.99 mL/kg |
| J | 02 | 100 μg/kg | 112.7 μg/mL | 0.89 mL/kg |

The dose volume for the vehicle and test substance was determined by the concentration of each dosing solution. Each rat received a single dose of test substance, positive control or vehicle, by subcutaneous injection.

Ten groups of animals (n=8 per group (n=4 for 5 treatment sample times)) received a subcutaneous dose, by injection, of test substance, vehicle or positive control to allow blood samples to be taken for analysis. The allocations are provided in Table 7.

TABLE 7
Allocations

| Animal Number | Treatment | Blood Sample Times |
|---|---|---|
| 1-4 | A | 0, 24, 48, 96 and 144 h |
| 5-8 | A | 12, 36, 72, 120 and 168 h |
| 9-12 | B | 0, 24, 48, 96 and 144 h |
| 13-16 | B | 12, 36, 72, 120 and 168 h |
| 17-20 | C | 0, 24, 48, 96 and 144 h |
| 21-24 | C | 12, 36, 72, 120 and 168 h |
| 25-28 | D | 0, 24, 48, 96 and 144 h |
| 29-32 | D | 12, 36, 72, 120 and 168 h |
| 33-36 | E | 0, 24, 48, 96 and 144 h |
| 37-40 | E | 12, 36, 72, 120 and 168 h |
| 41-44 | F | 0, 24, 48, 96 and 144 h |
| 45-48 | F | 12, 36, 72, 120 and 168 h |
| 49-52 | G | 0, 24, 48, 96 and 144 h |
| 53-56 | G | 12, 36, 72, 120 and 168 h |
| 57-60 | H | 0, 24, 48, 96 and 144 h |
| 61-64 | H | 12, 36, 72, 120 and 168 h |
| 65-68 | I | 0, 24, 48, 96 and 144 h |
| 69-72 | I | 12, 36, 72, 120 and 168 h |
| 73-76 | J | 0, 24, 48, 96 and 144 h |
| 77-80 | J | 12, 36, 72, 120 and 168 h |

For the initial samples, approximately 0.5 mL of whole blood was taken from the tail vein. For the fifth/final samples, the animals were anaesthetized with carbon dioxide and blood samples of maximal volume were taken by cardiac puncture. The time points for cardiac sampling were 144 and 168 hours post-dose. The samples were placed into EDTA coated collection vials, mixed immediately and then stored on ice. The animals were killed by cervical dislocation following the final blood sampling time point. The actual time of blood sampling was documented. The blood samples were immediately placed on ice and stored refrigerated at approximately 4° C. prior to analysis. Blood samples were analyzed within 48 hours of sampling. EDTA was used as an anticoagulant for the hematology samples.

The parameters measured and methodology for hematology are provided in Table 8.

TABLE 8
Parameters Measured and Methodology for Hematology

| PARAMETER | CODE | UNITS | METHOD |
|---|---|---|---|
| White Blood Count | WBC | ×10³/μL | Advia 120 |
| Neutrophils | Neut | ×10³/μL | Advia 120 |
| Lymphocytes | Lymph | ×10³/μL | Advia 120 |
| Eosinophils | Eosin | ×10³/μL | Advia 120 |
| Monocytes | Mono | ×10³/μL | Advia 120 |
| Basophils | Baso | ×10³/μL | Advia 120 |

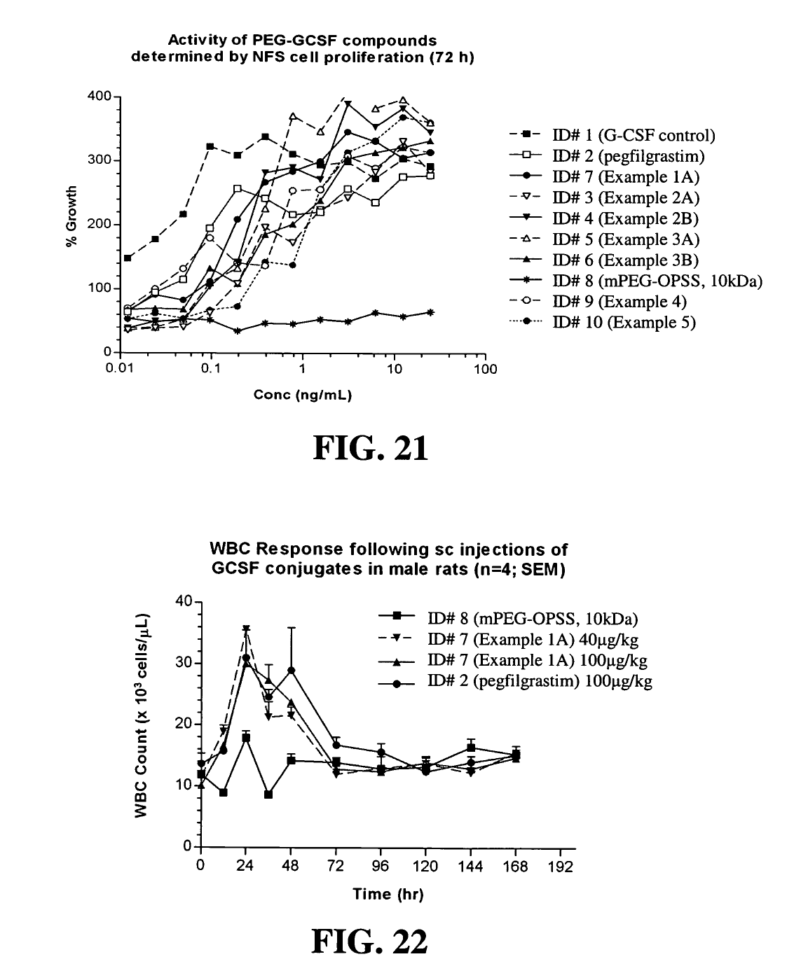

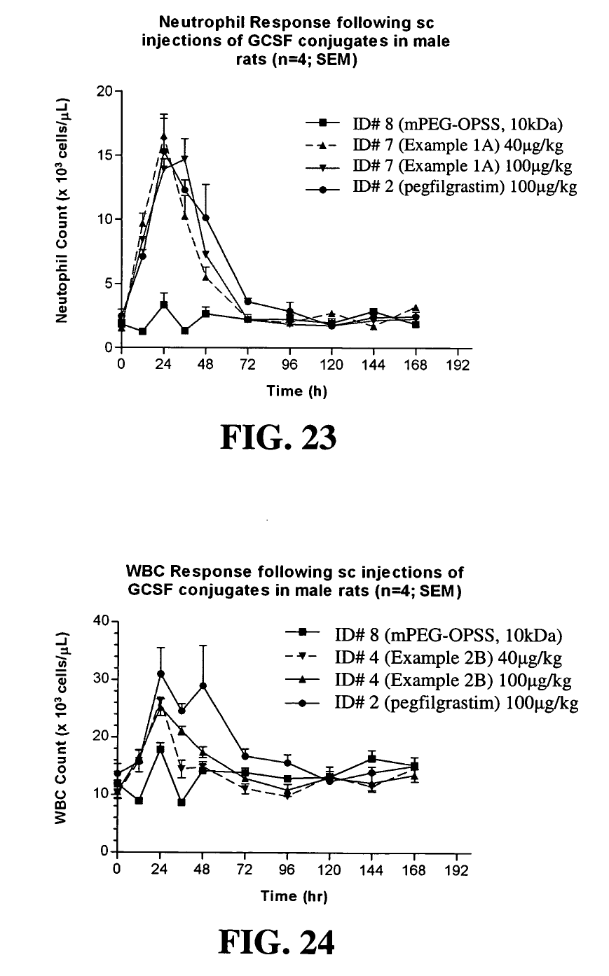

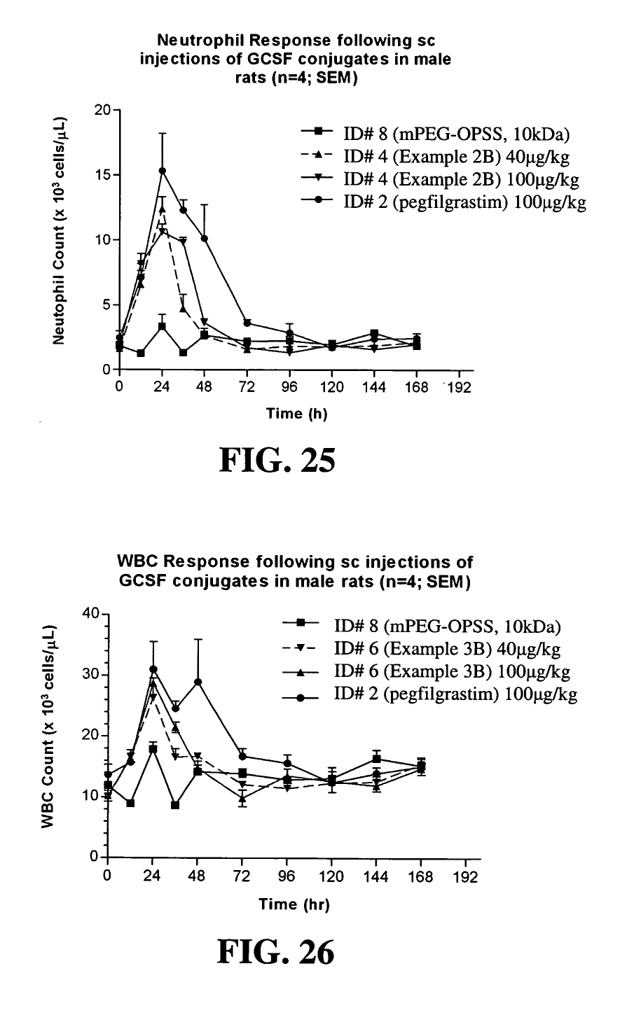

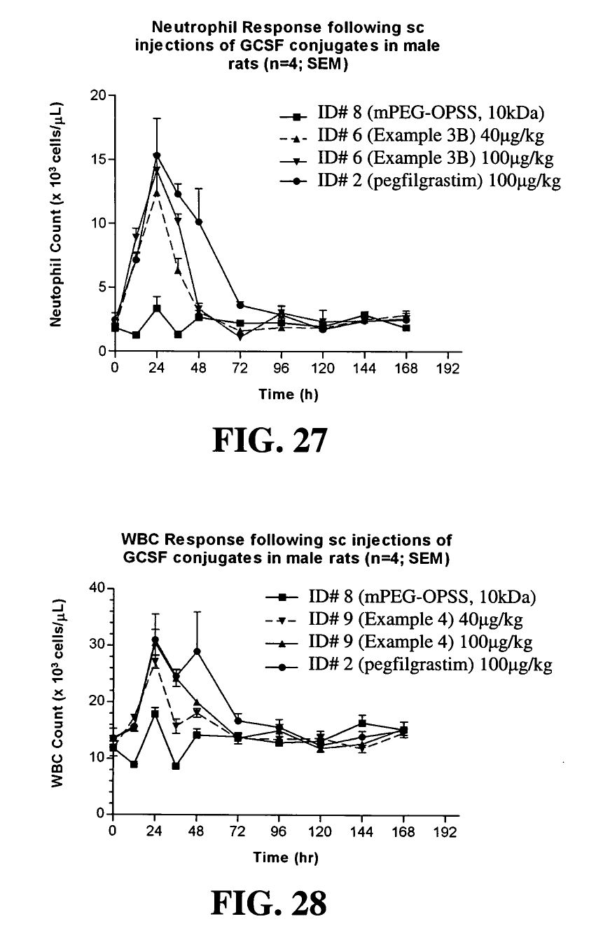

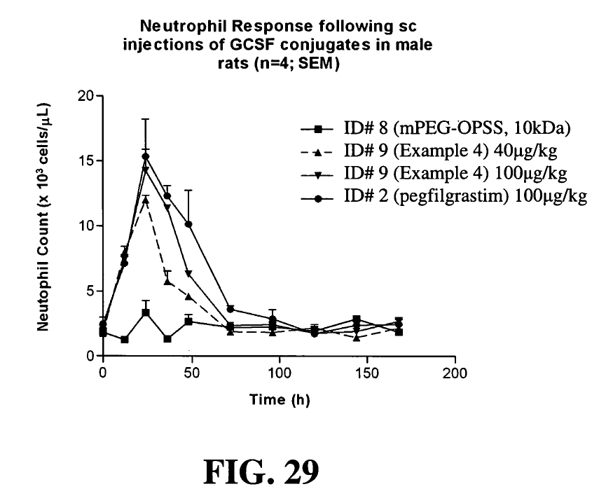

The activity of each tested compound is illustrated in the following FIG. 20 and FIG. 21. Each plot portrays the % growth in NFS cells as a function of concentration. The differences in the two figures are the length of incubation, i.e., 48 hours for FIG. 20 versus 72 hours for FIG. 21.

The results suggest that GCSF is active at lower concentrations when compared to each of the tested PEG-GCSF conjugates. In order to have a better comparison of the results in the plots, the data was normalized and directly compared to native GCSF. The activity in EC₅₀ (ng/mL) was calculated. The following table makes the comparisons.

TABLE 9
Activity Comparison Between G-CSF (ID# 1) and Tested Compounds

| | Activity in EC₅₀ | |
|---|---|---|
| ID# | (ng/mL) | Change |
| 1 | 0.012 | 0 |
| 2 | 0.097 | 8 x |
| 7 | 0.195 | 16 x |
| 3 | 0.39 | 32 x |
| 4 | 0.39 | 32 x |
| 5 | 0.39 | 32 x |
| 6 | 0.39 | 32 x |
| 8 | 0.00 | N/A |
| 9 | 0.78 | 64 x |
| 10 | 1.56 | 128 x |

The results suggest that pegfilgrastim (ID# 2) is 8 times less potent than native GCSG (ID# 1), the conjugate prepared in Example 1A (ID# 7) is 16 times less potent, and each of the conjugates prepared in Examples 2A, 2B, 3A, and 3B are 32 times less potent, i.e., a 1.5 log reduction. The releasable conjugates were not very potent because the PEGylation was believed to be non-selective. The releasable conjugates, however, are expected to release the active GCSF molecule, which in turn would have full activity as the native compound. As expected, the polymeric reagent control (ID# 8) did not have any activity.

It was also observed that native GCSF (ID# 1) and pegfilgrastim had better activity than the cysteine conjugated compounds at concentrations below 0.097 ng/mL. At concentrations above 0.097 ng/mL, however, the opposite was observed (i.e., the cell proliferation activity GCSF and pegfilgrastim was flat, while the PEG-GCSF samples showed continuous growth.

With respect to in vivo activity, the neutrophil and white blood cell counts were counted and compared. In each of the plots below the counts were plotted (for both doses) and compared against pegfilgrastim (N-terminal PEGylated GCSF). In all plots, a small but visible dose response was observed between the 40 μg/kg and 100 μg/kg doses.

In conclusions, the cysteine-based conjugates of GCSF showed positive activity in both in-vitro and in-vivo. The conjugate of Example 1A appeared to have activity that was closest to pegfilgrastim. There was no significant difference between the activities measured for the conjugates of Examples 2A, 2B, 3A and 3B. The releasable conjugates demonstrated positive activity as well.

What is claimed is:

1. A conjugate comprising a residue of a G-CSF moiety covalently attached via a degradable linkage, either directly or through a spacer moiety comprised of one or more atoms, to a water-soluble polymer.

2. The conjugate of claim 1, wherein the degradable linkage is a cleavable linkage.

3. The conjugate of claim 1, wherein an amino group of the G-CSF moiety serves as a site for covalent attachment.

4. The conjugate of claim 1, having the following structure: 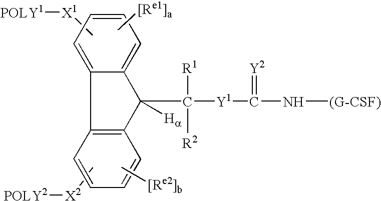
wherein:
POLY¹ is a first water-soluble polymer;
POLY² is a second water-soluble polymer;
X¹ is a first spacer moiety;
X² is a second spacer moiety;
H_{α} is an ionizable hydrogen atom;
R¹ is H or an organic radical;
R² is H or an organic radical;
(a) is either zero or one;
(b) is either zero or one;
R^{e1}, when present, is a first electron altering group;
R^{e2}, when present, is a second electron altering group;
Y¹ is O or S;
Y₂ is O or S; and
G-CSF is a residue of a G-CSF moiety.

5. The conjugate of claim 4, selected from the group consisting of 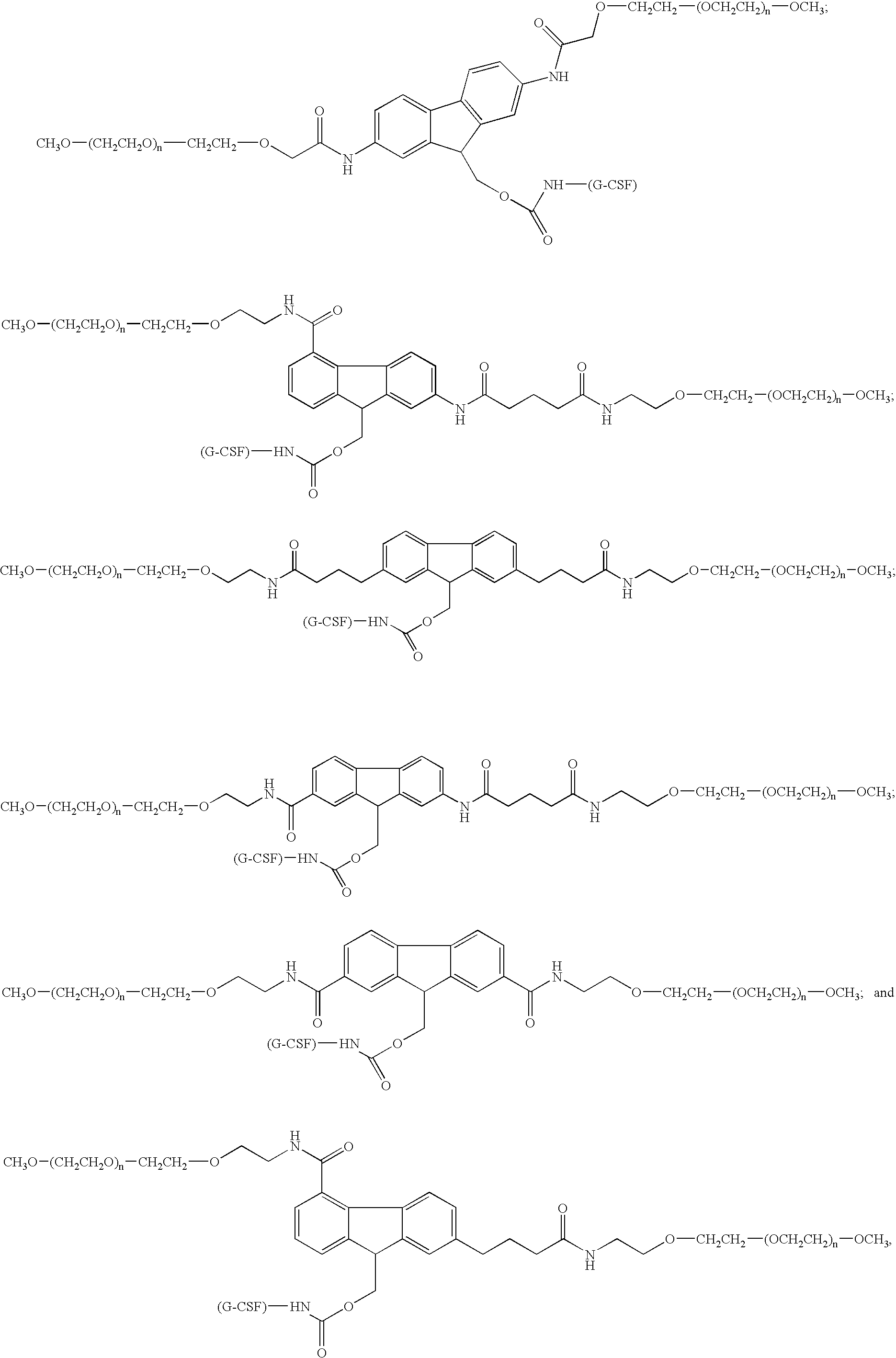
wherein (n) is 3 to 4,000 and G-CSF is a residue of a G-CSF moiety.

6. The conjugate of any one of claims 1, 2, 3, 4 and 5, wherein the G-CSF moiety is recombinant human G-CSF or is recombinant human G-CSF having an N-terminal methionyl residue.

7. A conjugate having the following structure:
POLY″-(X²)_{b}-POLY′-(X¹)ₐ-(G-CSF)
wherein:
POLY″ is a second water-soluble polymer;
POLY′ is a first water-soluble polymer
X¹, when present, is an optional spacer moiety comprised of one or more atoms;
X² is a spacer moiety comprised of one or more atoms;
(b) is one;
(a) is either zero or one; and
G-CSF is a residue of a G-CSF moiety.

8. The conjugate of claim 7, wherein POLY″ is a branched polymer

9. The conjugate of claim 8, wherein the branched polymer comprises a structure selected from the group consisting of: 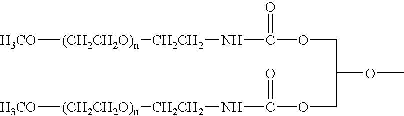
wherein each (n) is independently an integer having a value of from 3 to 4000; and 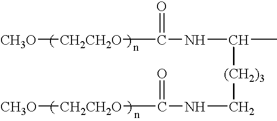
wherein each (n) is independently an integer having a value of from 3 to 4000.

10. The conjugate of claim 7, wherein POLY″ is a linear polymer.

11. The conjugate of claim 7, wherein a thiol group of the G-CSF moiety serves as a site for covalent attachment of the first water-soluble polymer or, when present, the optional spacer moeity.

12. The conjugate of claim 11, having the following structure:
CH₃—O(CH₂CH₂O)_{n′}—[CH₂]₂₋₈—S—S—[CH₂]₂₋₈—O(CH₂CH₂O)ₙ—[CH₂]₂₋₈—S——(G-CSF)
wherein (n) is an integer of from 2 to about 114, n′ is an integer from 2 to about 3,400, and G-CSF is a residue of a G-CSF moiety.

13. The conjugate of claim 11, having the following structure: 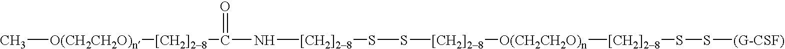
wherein (n) is an integer of from 2 to about 114, n′ is an integer from 2 to about 3,400, and G-CSF is a residue of a G-CSF moiety.

14. The conjugate of any one of claims 7, 8, 9, 10, 11, 12, and 13, wherein the G-CSF moiety is recombinant human G-CSF or is recombinant human G-CSF having an N-terminal methionyl residue.

15. A method for preparing a conjugate, the method comprising combining a polymeric reagent and a G-CSF moiety under conditions sufficient to result in the formation of a conjugate comprising a residue of a G-CSF moiety covalently attached, either directly or through a spacer moiety comprised of one or more atoms, to a water-soluble polymer, wherein the G-CSF moiety is covalently attached at a side chain of a cysteine residue, and further wherein the method (a) lacks a step introducing denaturing conditions, and (b) is carried out at a pH of less than 8.5 or lacks a step of adding a detergent.

16. The method of claim 15, wherein the cysteine residue corresponds to cysteine 17 of hG-CSF.

17. The method of any of claims 15 and 16, wherein the G-CSF moiety is either recombinant human G-CSF or is recombinant human G-CSF having an N-terminal methionyl residue.

18. A method for preparing a conjugate is provided, the method comprising adding a first polymeric reagent composition to a G-CSF moiety composition under conditions sufficient to result in a first conjugate composition comprising a first conjugate comprised of a residue of a G-CSF moiety covalently attached, either directly or through a first spacer moiety comprised of one or more atoms, to a first water-soluble polymer, and adding a second polymeric reagent composition to the first conjugate composition to result in a second conjugate composition comprising a second water-soluble polymer attached, either directly or through a second spacer moiety comprised of one or more atoms, to the first water-soluble polymer of the conjugate.

19. The composition prepared according to the method of claim 15.

20. The composition prepared according to the method of claim 18.
